(19) United States
(12) Patent Application Publication
(10) Pub. No.: US 2021/0169911 A1
(43) Pub. Date: Jun. 10, 2021

(54) PURINE AND PYRIMIDINE NUCLEOTIDES AS ECTO-5'-NUCLEOTIDASE INHIBITORS

(71) Applicants: The United States of America,as represented by the Secretary,Department of Health and Human Services, Bethesda, MD (US); University of Bonn, Bonn (DE); University of Münster, Münster (DE)

(72) Inventors: Kenneth A. Jacobson, Silver Spring, MD (US); Anna Junker, Senden (DE); Mirko Scortichini, Rockville, MD (US); Clemens Dobelmann, Münster (DE); Christian P. Renn, Bonn (DE); Vigneshwaran Namasivayam, Bonn (DE); Riham Mohammed Idris, Bad Neuenar-Ahrweiler (DE); Christa E. Müller, Bonn (DE)

(73) Assignees: The United States of America,as represented by the Secretary,Department of Health and Human Services, Bethesda, MD (US); University of Bonn, Bonn (DE); University of Münster, Münster (DE)

(21) Appl. No.: 17/269,094

(22) PCT Filed: Aug. 16, 2019
(86) PCT No.: PCT/US2019/046931

§ 371 Date: Feb. 17, 2021

Related U.S. Application Data
(60) Provisional application No. 62/719,492, filed on Aug. 17, 2018.

(51) Int. Cl. A61K 31/7068; A61K 49/00; A61K 51/04; A61K 39/395; C07H 19/14; C07H 19/10

(52) U.S. Cl. CPC A61K 31/7068; A61K 49/0052; A61K 51/0491; A61K 39/3955; A61K 49/0021; C07H 19/10; A61K 49/0041; A61K 49/0028; A61K 49/0036; C07H 19/14

(57) ABSTRACT

Disclosed is a compound of formula (I), wherein Q, U, T, A, a, b, c, and n are as defined herein. Also disclosed are methods of inhibiting ecto-5′-nucleotidase, inhibiting suppression of an antitumor immune response, inhibiting tumor growth of a cancerous tumor, inhibiting metastasis of cancer in a mammal afflicted with cancer, synergistically enhancing a response of a mammal afflicted with cancer undergoing treatment with an immunotherapeutic anti-cancer agent, potentiating an activity of an inhibitor of nicotinamide phosphoribosyltransferase in a mammal undergoing treatment of a mammal with the inhibitor, and treating preeclampsia in a mammal in need thereof, comprising administering to an animal an effective amount of a compound of formula (I).

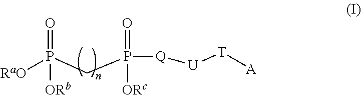

### CROSS-REFERENCE TO A RELATED APPLICATION

This patent application claims the benefit of U.S. Provisional Patent Application No. 62/719,492, filed Aug. 17, 2018, the disclosure of which is incorporated herein by reference in its entirety for all purposes.

### STATEMENT REGARDING FEDERALLY SPONSORED RESEARCH OR DEVELOPMENT

This invention was made with Government support under Grant Number ZIADK031117 awarded by the National Institute of Diabetes & Digestive & Kidney Diseases. The Government has certain rights in this invention.

### BACKGROUND OF THE INVENTION

Ecto-5′-nucleotidase (ecto-5′-NT, eN, CD73, EC 3.1.3.5) is a glycosylphosphatidylinositol (GPI)-linked cell surface enzyme that dephosphorylates extracellular nucleoside monophosphates (1, 2, 12). The enzyme can be cleaved from its GPI linker and is also present in a soluble active form in serum. The vertebrate enzyme hydrolyzes selectively adenosine 5′-monophosphate (AMP) over adenosine 2′- or 3′-monophosphates leading to elevated extracellular concentrations of adenosine (1). X-ray crystallographic structures of the enzyme in complex with either inhibitor or substrate have been reported (3, 10). There is a marked conformational rearrangement of the structure as catalysis occurs. Two conformational classes have been determined, an open and a closed form.

Although the substrate, AMP, is not a potent agonist of adenosine receptors (ARs), the enzymatic product, adenosine, activates four AR subtypes (A_{F}AR, A₂AR, A_{2B}AR, and A_{3A}R) (22). One of the important activities of adenosine is the suppression of inflammation (22). Thus, CD73 upregulation and increased production of adenosine are beneficial in chronic inflammatory diseases. Co-expression of CD73 and the A_{2A}AR found in many tissues including the brain and immune cells, especially when inflammation is present, allows the concerted activation of this receptor (2, 5, 6). However, in the tumor microenvironment, elevated CD73 and adenosine counteract the body's immune defense against the tumor (7, 8, 11, 13, 18, 21). This is particularly important in cancer immunotherapy, for which co-administration of either an A2AAR antagonist or an inhibitor of CD73 offers synergistic anti-tumor activity. CD73 inhibitor also potentiates the in vivo anticancer effect of inhibitors of nicotinamide phosphoribosyltransferase (20), which is required for the biosynthesis of intracellular NAD⁺.

CD73 inhibitors have potential applications as novel therapeutics for melanomas as well as lung, prostate, and breast cancers (26-28). CD73 favors tumor cell growth by inhibiting T cell activity and promoting angiogenesis (29, 30). It has been shown that inhibition of CD73 with a monoclonal antibody, siRNA, or small molecules delays tumor growth and metastasis (11, 12). The same effects were observed in CD73 knockout mice due to decreased adenosine production (13). High CD73 expression has been reported in triple-negative breast cancers, and it has also been demonstrated that targeted blockage of CD73 significantly prolonged the survival in anthracycline-resistant animal models of cancer (28).

Antibodies against CD73 are currently undergoing clinical trials for cancer therapy (34, 35). Assays of CD73 have been developed (4, 14), leading to reports of a variety of diverse inhibitors of CD73 (9, 15, 16, 17, 19). Among the first potent inhibitors to be identified was adenosine-5′-O-[(phosphonomethyl)phosphonic acid (I, α,β-methylene-ADP, AOPCP) (23). Also, various anthraquinone (15) and sulfonamide (16) derivatives were found to be competitive inhibitors, while polyphenols (24) and polyoxometalates (POMs) (25) are non-competitive CD73 inhibitors. Recently, adenine nucleotide analogs of I that display low nM potency in inhibition of CD73 were identified (9). However, adenine adenine nucleotide I and its congeners, if hydrolyzed to the parent nucleoside, could potentially activate ARs via liberation of adenine and/or analogs thereof.

Thus, there remains an unmet need for novel inhibitors of CD73 and methods of treatment of diseases and disorders responsive to inhibition of CD73.

### BRIEF SUMMARY OF THE INVENTION

In accordance with an embodiment, the invention provides a compound of formula (I):

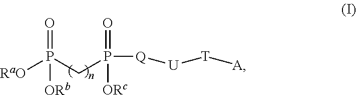

wherein Q is O, S, CH₂, or NH;
U is O, CH₂, C₂H₄, NH or S;
n is an integer of from 1 to 6;
R^{a}, R^{b} and R^{c} are independently H, C₁-C₆ alkyl, C₆-C₁₀ aryl, C₁-C₆ alkylcarbonyl, C₆-C₁₀ heteroaryl-C₁-C₆ alkyl, C₆-C₁₀ heteroaryl, or C₆-C₁₀ arylcarbonyl;
T is

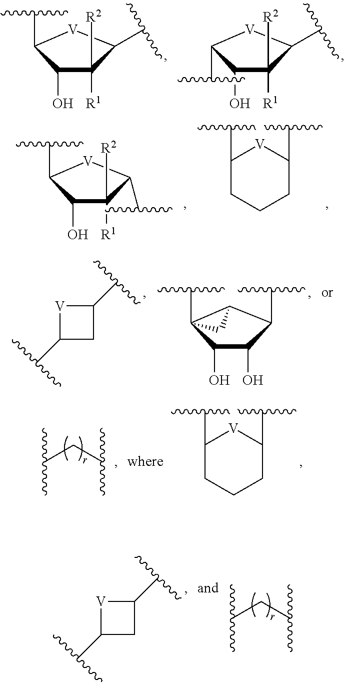

are optionally substituted with 1, 2, or 3 hydroxyl groups and r is an integer from 2 to about 6,
V is O, S, CH₂ or NH;
A is

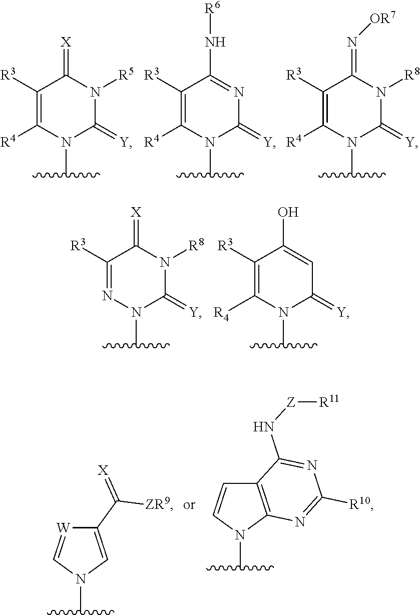

wherein X, Y, and Z are independently O, S, NH or C₁-C₆ alkylenyl, and W is independently N or CH,
R¹ and R² are independently H, OH, SH, C₁-C₆ alkoxy, C₁-C₆ thioalkoxy, NH₂, C₁-C₆ alkylamino, N₃, or halo;
R³, R⁴ and R¹⁰ are independently H, halo, C₁-C₆ alkyl, C₁-C₆ aryl, NH₂, N₃, C₁-C₆ alkynyl, C₆-C₁₀ aryl-C₁-C₆ alkylenyl, 1-halo-1-vinyl, C₆-C₁₀ heteroaryl-C₁-C₆ alkyl, C₆-C₁₀ heteroaryl or C₆-C₁₀ arylcarbonyl,
R⁵ to R⁸ and R¹¹ are independently H, C₁-C₆ alkyl, C₆-C₁₀ aryl-C₁-C₆ alkyl, C₆-C₁₀ aryl-C₁-C₆ alkylenyl, C₁-C₆ alkylcarbonyl, C₆-C₁₀ heteroaryl-C₁-C₆ alkyl, C₆-C₁₀ heteroaryl or C₆-C₁₀ arylcarbonyl,
R⁹ is C₁-C₆ alkyl, C₆-C₁₀ aryl-C₁-C₆ alkylenyl, C₁-C₆ alkylcarbonyl, C₆-C₁₀ heteroaryl-C₁-C₆ alkyl, C₆-C₁₀ heteroaryl or C₆-C₁₀ arylcarbonyl,
wherein aryl at each occurrence is optionally substituted with one or more substituents selected from the group consisting of C₁-C₆ alkyl, C₂-C₆ alkynyl, halo, trifluoromethyl, OH, SH, NH₂, SO₂NH₂, C₁-C₆ hydroxyalkyl, C₁-C₆ alkoxy, sulfonyloxy, C₁-C₆ carboxyalkyl, carboxy, carboxamide, C₁-C₆ sulfonyloxyalkyl, arylcarbonyl, CONH(CH)ₚNH₂,

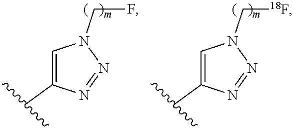

and any combination thereof, wherein heteroaryl is optionally substituted with one or more substituents selected from C₁-C₆ alkyl, halo, trifluoromethyl, C₁-C₆ hydroxyalkyl, C₁-C₆ alkoxy, sulfonyloxy, C₁-C₆ carboxyalkyl, C₁-C₆ sulfonyloxyalkyl, arylcarbonyl,

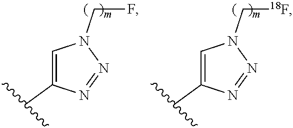

and any combination thereof,
wherein m is an integer of from 2 to about 10,
wherein p is an integer of from 2 to about 10,
   or a pharmaceutically acceptable salt thereof.

The invention further provides a compound of the formula:

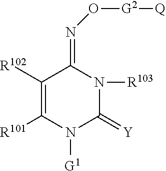

wherein R¹⁰¹ and R¹⁰² are independently H, halo, C₁-C₆ alkyl, C₁-C₆ aryl, NH₂, N₃, C₁-C₆ alkynyl, C₆-C₁₀ aryl-C₁-C₆ alkylenyl, 1-halo-1-vinyl, C₆-C₁₀ heteroaryl-C₁-C₆ alkyl, C₆-C₁₀ heteroaryl or C₆-C₁₀ arylcarbonyl,
R¹⁰³ is H, C₁-C₆ alkyl, C₆-C₁₀ aryl-C₁-C₆ alkyl, C₆-C₁₀ aryl-C₁-C₆ alkylenyl, C₁-C₆ alkylcarbonyl, C₆-C₁₀ heteroaryl-C₁-C₆ alkyl, C₆-C₁₀ heteroaryl or C₆-C₁₀ arylcarbonyl,
G¹ is

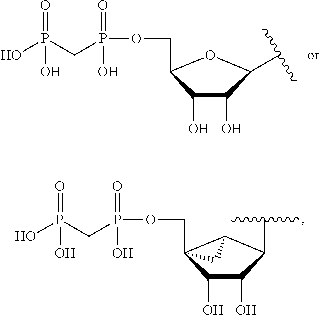

G² is

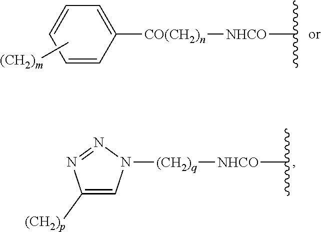

m, n, p, and q are independently integers of from 1 to about 20, and
Q is a fluorophore moiety;
   or a pharmaceutically acceptable salt thereof.

The invention also provide a composition comprising a compound of an embodiment of the invention or pharmaceutically acceptable salt thereof and a pharmaceutically acceptable carrier.

The invention further provides a method of inhibiting ecto-5′-nucleotidase in a mammal in need thereof, comprising administering to the mammal a compound of the invention or pharmaceutically acceptable salt thereof, in an amount effective to inhibit ecto-5′-nucleotidase in the mammal.

The invention further provides a method of inhibiting suppression of an antitumor immune response in a mammal in need thereof, comprising administering to the mammal a compound of the invention or pharmaceutically acceptable salt thereof, in an amount effective to suppress the antitumor immune response in the mammal.

The invention additionally provides a method of inhibiting tumor growth of a cancerous tumor in a mammal in need thereof, comprising administering to the mammal a compound of the invention or pharmaceutically acceptable salt thereof, in an amount effective to inhibit growth of the cancerous tumor in the mammal.

The invention also provides a method of inhibiting metastasis of cancer in a mammal in need thereof, comprising administering to the mammal a compound of the invention or pharmaceutically acceptable salt thereof, in an amount effective to inhibit metastasis of cancer in the mammal.

The invention additionally provides a method of synergistically enhancing a response of a mammal afflicted with cancer undergoing treatment with an immunotherapeutic anti-cancer agent in a mammal in need thereof, comprising administering to the mammal a compound of the invention or pharmaceutically acceptable salt thereof, in an amount effective to synergistically enhance a response of a mammal afflicted with cancer undergoing treatment with the immunotherapeutic anti-cancer agent in the mammal.

The invention further provides a method of potentiating an activity of an inhibitor of nicotinamide phosphoribosyltransferase in a mammal in need thereof, comprising administering to the mammal a compound of the invention or pharmaceutically acceptable salt thereof, in an amount effective to potentiate the activity of an inhibitor of nicotinamide phosphoribosyltransferase in the mammal.

The invention also provides a method treating preeclampsia in in a mammal in need thereof, comprising administering to the mammal a compound of the invention or pharmaceutically acceptable salt thereof, in an amount effective to treat preeclampsia in the mammal.

The invention additionally provides method of treating cancer in a mammal comprising administering to the animal an effective amount of a compound of any one of claims 1-26, wherein the cancer is lung cancer, prostate cancer, triple-negative breast cancer, melanoma, leukemia, or thyroid cancer.

### BRIEF DESCRIPTION OF THE DRAWINGS

FIG. 1 shows a comparison of pKᵢ values of compounds in accordance with embodiments of the invention, namely, compounds 4l, 7f 9e, 9h, 9d and 9g, wherein the pKᵢ values were determined at recombinant soluble rat and human and at membrane bound native CD73.

FIG. 2 shows a Synthesis of adenosine 2a-g and uridine derivatives 4a-y. Reagents and conditions: (a) DCC (3 eq), methylene diphosphonic acid (1.5 eq.), DMF, room temp, 3-24 h; for compounds 4w and 4x: DCC (3 eq), ethylene diphosphonic acid (1.5 eq.), DMF, room temp, 3 h (b) methylenebis(phosphonic dichloride) (3 eq.), trimethyl phosphate, 0° C., 30 min, then triethylammonium hydrogencarbonate buffer pH 8.4-8.6, rt, 30 min.

FIG. 3 shows a synthesis of cytosine-derived 5′-O-[(phosphonomethyl)phosphonic acid] derivatives 7a-f and 9a-i. Reagents and conditions: (a) DCC (3 eq), methylene diphosphonic acid (1.5 eq.), DMF, room temp, 3-24 h; (b) methylenebis(phosphonic dichloride) (3 eq.), trimethyl phosphate, 0° C., 30 min, then triethylammonium hydrogencarbonate buffer pH 8.4-8.6, rt, 30 min: (c) R₃—O—NH₂xHCl, pyridine, 80° C., 12 h. (d) alkyl iodide (1.5 eq.), K₂CO₃ (1.7 eq.) in DMF/acetone (1:1) at 50° C. for 3 d.

FIG. 4 shows a synthesis of compounds 105-115. Reagents and Conditions: i. PBr₃, THF, rt, 12 h; ii. (Boc)₂NOH¹, DBU, DMF, 50° C., 2 h; iii. HCl 4N dioxane sol., rt, 12 h; iv. MeI, Dmac, rt, 4 h; v. desired benzylhydroxylamine (25-35), pyridine, 80° C., 12 h; vi. methylenebis(phosphonic dichloride), (CH₃)₃PO₄, 0° C., 3 h.

FIG. 5 shows a shows a synthetic route for the preparation of derivatives 116-119.

FIG. 6 shows a synthesis of triazole compound 120. Reagents and Conditions: i. NaN₃, DMSO, 2 days, rt; ii. Sodium ascorbate, CuSO₄, THF/H₂O (1:1), 12 h, rt; methylenebis(phosphonic dichloride), (CH₃)₃PO₄, 0° C., 3 h.

FIG. 7 shows a synthesis of two (N)-methanocarba-5′-O-α,β-methylenediphosphates 121 and 122. Reagents and Conditions: i. Acetic anhydride, Et₃N, DMAP, CH₃CN, 2 h, rt; ii. TPSCl, Et₃N, DMAP, CH₃CN, rt, 18 h; iii. p-Cl—O-benzylhydroxylamine hydrochloride, Et₃N, CH₃CN, rt, 18 h; iv. MeI, K₂CO₃, Dmac, 18 h, rt; v. NH₃/MeOH, rt, 12 h; vi. methylenebis(phosphonic dichloride), (CH₃)₃PO₄, 0° C., 3 h.

FIG. 8 shows a shows the introduction of a ¹⁸F to compound 20. Reagents and conditions: CuSO₄, ascorbate, THF/H₂O (1:1), rt

FIG. 9 shows the structures of fluorophore moieties.

FIG. 10 shows the inhibition of the catalytic activity of CD73 in human tonsillary tissue.

### DETAILED DESCRIPTION OF THE INVENTION

In an embodiment, the invention provides a compound of formula (I):

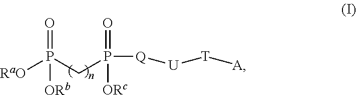

wherein Q is O, S, CH₂, or NH;
U is O, CH₂, C₂H₄, NH or S;
n is an integer of from 1 to 6;
R^{a}, R^{b} and R^{c} are independently H, C₁-C₆ alkyl, C₆-C₁₀ aryl, C₁-C₆ alkylcarbonyl, C₆-C₁₀ heteroaryl-C₁-C₆ alkyl, C₆-C₁₀ heteroaryl, or C₆-C₁₀ arylcarbonyl;

T is

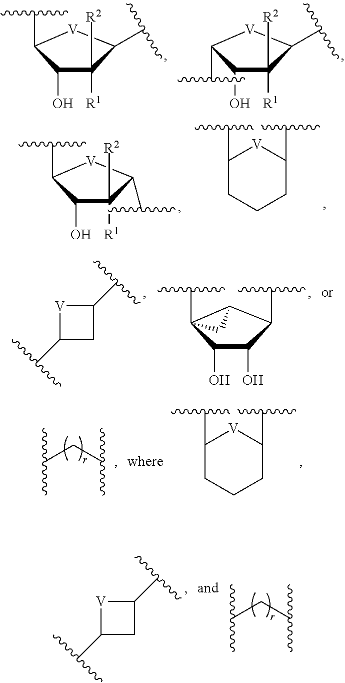

are optionally substituted with 1, 2, or 3 hydroxyl groups and r is an integer from 2 to about 6,
V is O, S, CH₂ or NH;
A is

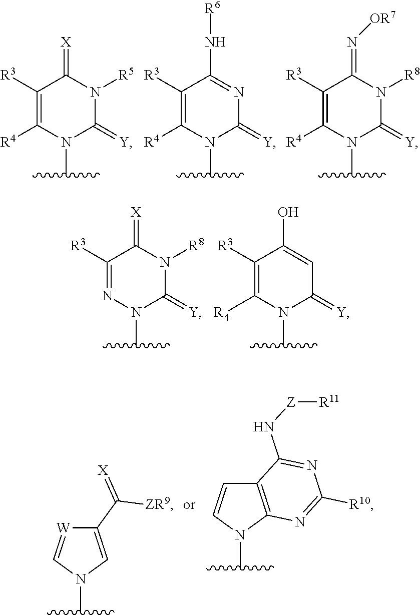

wherein X, Y, and Z are independently O, S, NH or C₁-C₆ alkylenyl, and W is independently N or CH,
R¹ and R² are independently H, OH, SH, C₁-C₆ alkoxy, C₁-C₆ thioalkoxy, NH₂, C₁-C₆ alkylamino, N₃, or halo;
R³, R⁴ and R¹⁰ are independently H, halo, C₁-C₆ alkyl, C₁-C₆ aryl, NH₂, N₃, C₁-C₆ alkynyl, C₆-C₁₀ aryl-C₁-C₆ alkylenyl, 1-halo-1-vinyl, C₆-C₁₀ heteroaryl-C₁-C₆ alkyl, C₆-C₁₀ heteroaryl or C₆-C₁₀ arylcarbonyl,
R⁵ to R⁸ and R¹¹ are independently H, C₁-C₆ alkyl, C₆-C₁₀ aryl-C₁-C₆ alkyl, C₆-C₁₀ aryl-C₁-C₆ alkylenyl, C₁-C₆ alkylcarbonyl, C₆-C₁₀ heteroaryl-C₁-C₆ alkyl, C₆-C₁₀ heteroaryl or C₆-C₁₀ arylcarbonyl,
R⁹ is C₁-C₆ alkyl, C₆-C₁₀ aryl-C₁-C₆ alkylenyl, C₁-C₆ alkylcarbonyl, C₆-C₁₀ heteroaryl-C₁-C₆ alkyl, C₆-C₁₀ heteroaryl or C₆-C₁₀ arylcarbonyl,
wherein aryl at each occurrence is optionally substituted with one or more substituents selected from the group consisting of C₁-C₆ alkyl, C₂-C₆ alkynyl, halo, trifluoromethyl, OH, SH, NH₂, SO₂NH₂, C₁-C₆ hydroxyalkyl, C₁-C₆ alkoxy, sulfonyloxy, C₁-C₆ carboxyalkyl, carboxy, carboxamide, C₁-C₆ sulfonyloxyalkyl, arylcarbonyl, CONH(CH)ₚNH₂,

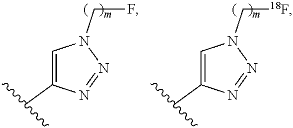

and any combination thereof, wherein heteroaryl is optionally substituted with one or more substituents selected from C₁-C₆ alkyl, halo, trifluoromethyl, C₁-C₆ hydroxyalkyl, C₁-C₆ alkoxy, sulfonyloxy, C₁-C₆ carboxyalkyl, C₁-C₆ sulfonyloxyalkyl, arylcarbonyl,

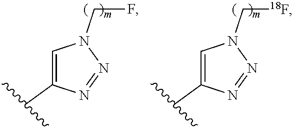

and any combination thereof,
wherein m is an integer of from 2 to about 10,
wherein p is an integer of from 2 to about 10,
with the proviso that when R^{a}, R^{b}, and R^{c} are all H, n is 1, Q is O, U is CH₂, T is

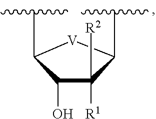

V is O, R¹ is OH, R² is H, and A is

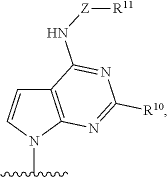

R⁵ is not H, methyl, ethyl, or benzyl and R¹⁰ is not H,
   or a pharmaceutically acceptable salt thereof.

Referring now to terminology used generically herein, the term “alkyl” means a straight-chain or branched alkyl substituent containing from, for example, 1 to 6 carbon atoms, preferably from 1 to about 4 carbon atoms, more preferably from 1 to 2 carbon atoms. Examples of such substituents include methyl, ethyl, propyl, isopropyl, n-butyl, sec-butyl, isobutyl, tert-butyl, pentyl, isoamyl, and hexyl. The term “alkylenyl” refers to an alkyl group substituted at two positions on the group. An example of an alkylenyl group is 1,3-propylenyl. The term “alkenyl” refers to a disubstituted group containing a C═C double bond. Examples of alkenyl substituents include ethylenyl, propylenyl, butylenyl. The term “alkynyl” refers to a group containing a C═C triple bond. Examples of alkylynyl substituents include ethylynyl, propylynyl, butylynyl.

The term “heteroaryl” refers to a monocyclic or bicyclic 5- or 6-membered ring system as described herein, wherein the heteroaryl group is unsaturated and satisfies Bickel's rule. Non-limiting examples of suitable heteroaryl groups include furanyl, thiopheneyl, pyrrolyl, pyrazolyl, imidazolyl, 1,2,3-triazolyl, 1,2,4-triazolyl, isoxazolyl, oxazolyl, isothiazolyl, thiazolyl, 1,3,4-oxadiazol-2-yl, 1,2,4-oxadiazol-2-yl, 5-methyl-1,3,4-oxadiazole, 3-methyl-1,2,4-oxadiazole, pyridinyl, pyrimidinyl, pyrazinyl, pyridazinyl, triazinyl, benzofuranyl, benzothiopheneyl, indolyl, quinolinyl, isoquinolinyl, benzimidazolyl, benzoxazolinyl, benzothiazolinyl, and quinazolinyl. The heteroaryl group is optionally substituted with 1, 2, 3, 4, or 5 substituents as recited herein such as with C₁-C₆ alkyl, halo, trifluoromethyl, C₁-C₆ hydroxyalkyl, C₁-C₆ alkoxy, sulfonyloxy, C₁-C₆ carboxyalkyl, C₁-C₆ sulfonyloxyalkyl, arylcarbonyl, and any combination thereof and the like, wherein the optional substituent can be present at any open position on the heteroaryl group

The term “alkylcarbonyl,” as used herein, refers to an alkyl group linked to a carbonyl group and further linked to a molecule via the carbonyl group, e.g., alkyl-C(═O)—. The term “arylcarbonyl,” as used herein, refers to an aryl group linked to a carbonyl group and further linked to a molecule via the carbonyl group, e.g., aryl-C(═O)—. The term “alkoxycarbonyl,” as used herein, refers to an alkoxy group linked to a carbonyl group and further linked to a molecule via the carbonyl group, e.g., alkyl-O—C(═O)—.

The term “halo” or “halogen,” as used herein, means a substituent selected from Group VIIA, such as, for example, fluorine, bromine, chlorine, and iodine.

The term “aryl” refers to an unsubstituted or substituted aromatic carbocyclic substituent, as commonly understood in the art, and the term “C₆-C₁₀ aryl” includes phenyl and naphthyl. It is understood that the term aryl applies to cyclic substituents that are planar and comprise 4n+2 π electrons, according to Hückel's Rule. The substituted aryl group is an aryl group substituted with 1, 2, 3, 4, or 5 substituents as recited herein, for example, C₁-C₆ alkyl, halo, trifluoromethyl, OH, SH, NH₂, SO₂NH₂, C₁-C₆ hydroxyalkyl, C₁-C₆ alkoxy, sulfonyloxy, C₁-C₆ carboxyalkyl, C₁-C₆ sulfonyloxyalkyl, arylcarbonyl, and any combination thereof.

The term “acyl” refers to an alkylcarbonyl (R—C(═O)—) substituent. The term “aminosulfonyl” refers to a group of the structure: H₂NSO₂—. The term “sulfonyloxy” refers to a group of the structure: —SO₃H. The term “sulfonyloxyalkyl” refers to a group of the structure: alkyl-SO₂—. The term “hydroxyalkyl” refers to an alkyl group substituted with a hydroxyl group. The term “alkoxy” refers to an alkyl group substituted with an O atom: R—O—. The term “thioalkoxy” refers to an alkyl group substituted with an S atom: R—S—. The term “alkylamino” refers to an alkyl group substituted with an NH group: R—NH—. The term “carboxyalkyl” refers to a group of the structure: —C(═O)O—R.

Whenever a range of the number of atoms in a structure is indicated (e.g., a C₁-C₁₂, C₁-C₈, C₁-C₆, C₁-C₄, or C₂-C₁₂, C₂-C₈, C₂-C₆, C₂-C₄ alkyl, alkenyl, alkynyl, etc.), it is specifically contemplated that any sub-range or individual number of carbon atoms falling within the indicated range also can be used. Thus, for instance, the recitation of a range of 1-8 carbon atoms (e.g., C₁-C₈), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, alkylamino, etc.) referenced herein encompasses and specifically describes 1, 2, 3, 4, 5, 6, 7, 8, 9, 10, 11, and/or 12 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 1-9 carbon atoms, 1-10 carbon atoms, 1-11 carbon atoms, 1-12 carbon atoms, 2-3 carbon atoms, 2-4 carbon atoms, 2-5 carbon atoms, 2-6 carbon atoms, 2-7 carbon atoms, 2-8 carbon atoms, 2-9 carbon atoms, 2-10 carbon atoms, 2-11 carbon atoms, 2-12 carbon atoms, 3-4 carbon atoms, 3-5 carbon atoms, 3-6 carbon atoms, 3-7 carbon atoms, 3-8 carbon atoms, 3-9 carbon atoms, 3-10 carbon atoms, 3-11 carbon atoms, 3-12 carbon atoms, 4-5 carbon atoms, 4-6 carbon atoms, 4-7 carbon atoms, 4-8 carbon atoms, 4-9 carbon atoms, 4-10 carbon atoms, 4-11 carbon atoms, and/or 4-12 carbon atoms, etc., as appropriate). Similarly, the recitation of a range of 6-10 carbon atoms (e.g., C₆-C₁₀) as used with respect to any chemical group (e.g., aryl) referenced herein encompasses and specifically describes 6, 7, 8, 9, and/or 10 carbon atoms, as appropriate, as well as any sub-range thereof (e.g., 6-10 carbon atoms, 6-9 carbon atoms, 6-8 carbon atoms, 6-7 carbon atoms, 7-10 carbon atoms, 7-9 carbon atoms, 7-8 carbon atoms, 8-10 carbon atoms, and/or 8-9 carbon atoms, etc., as appropriate).

In certain embodiments, Q is O, U is CH₂, and T is

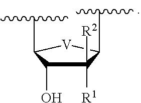

In certain embodiments, A is

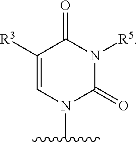

In certain of these embodiments, R¹ is H, OH, NH₂, N₃, C₁-C₆ alkoxy or halo and R² is H or halo.

In any of the above embodiments, R³ is H, halo, methyl, ethynyl, or 1-chloro-1-vinyl.

In any of the above embodiments, R⁵ is H, methyl, ethyl, propyl or benzyl.

In particular embodiments, the compound is selected from the group consisting of:

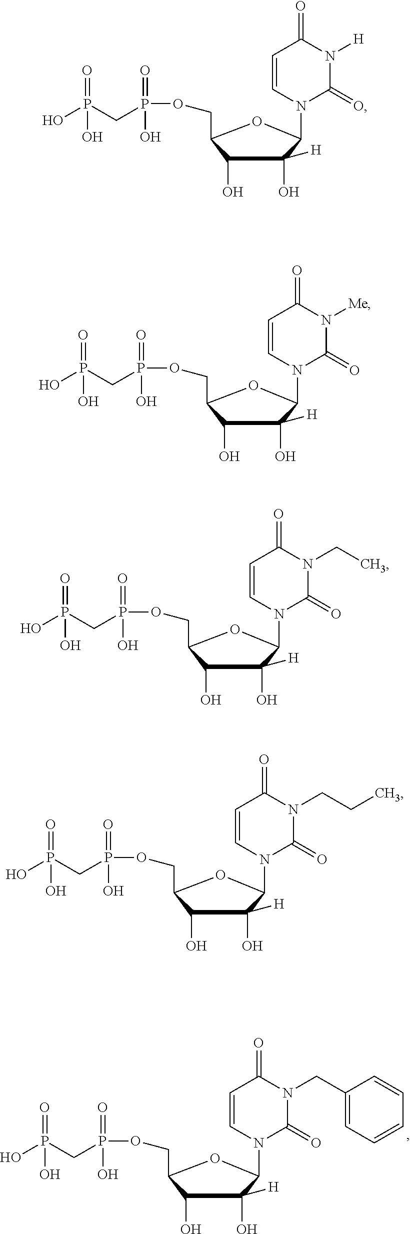

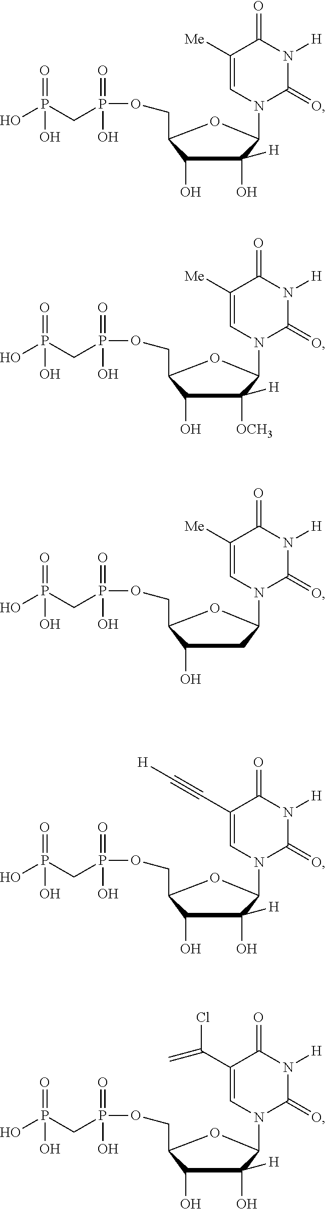

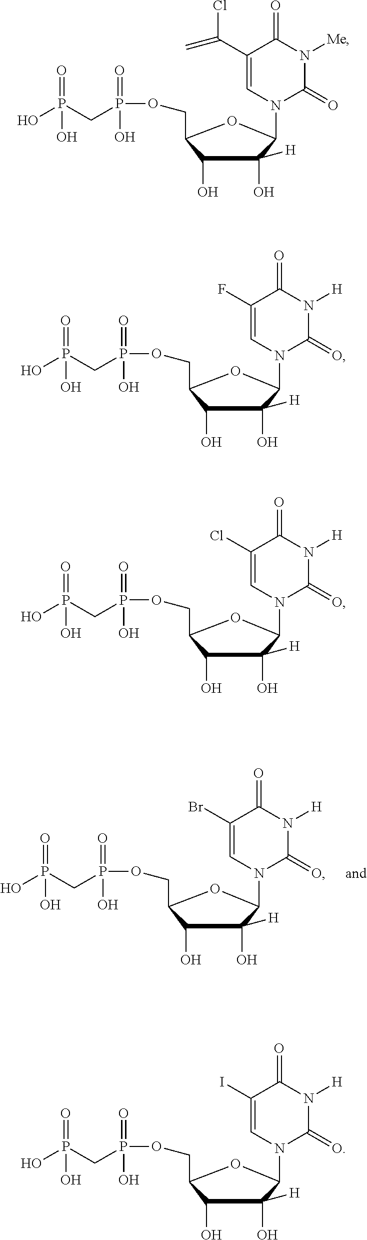

In certain embodiments, R¹ is H and R² is F or OH.

In a particular embodiments compound is selected from the group consisting of:

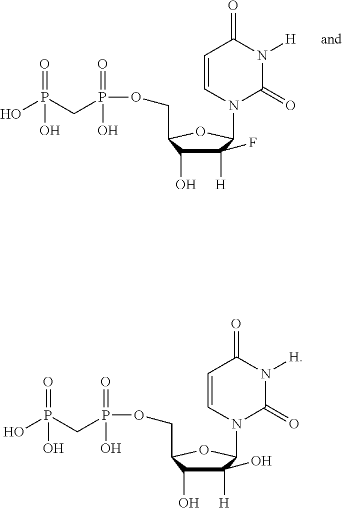

In certain embodiments, T is

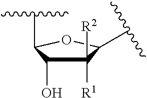

### and A is

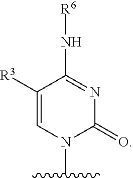

In certain of these embodiments, R¹ is OH or H, and R² is H.

In certain of these embodiments, R³ is H, halo or C₁-C₆ alkyl.

In any of the above embodiments, R⁶ is H, C₁-C₆ alkyl, C₁-C₆-alkylcarbonyl or C₆-C₁₀ arylcarbonyl.

In particular embodiments, the compound is selected from the group consisting of:

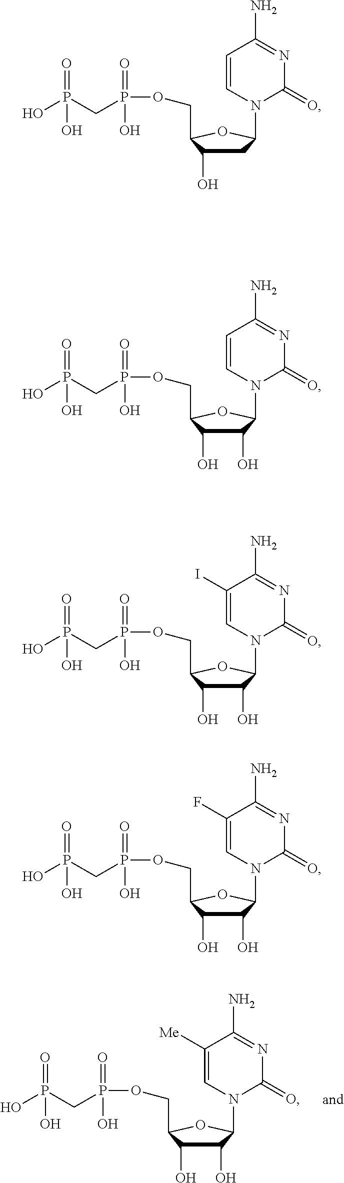

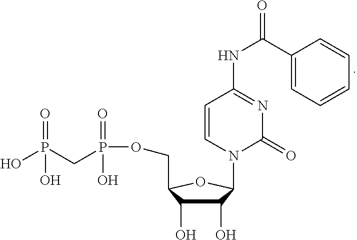

In certain embodiments, T is

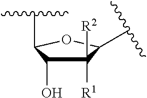

### and A is

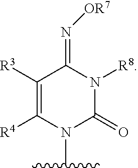

In certain of these embodiments, R¹ is H or OH and R² is H.

In certain of these embodiments, R³ is H, halo or C₁-C₆ alkyl.

In certain of these embodiments, R⁷ is H, C₁-C₆ alkyl, C₆-C₁₀ aryl-C₁-C₆ alkyl, C₆-C₁₀ aryl-C₁-C₆ alkylenyl, C₁-C₆ alkylcarbonyl, C₆-C₁₀ heteroaryl-C₁-C₆ alkyl, C₆-C₁₀ heteroaryl or C₆-C₁₀ arylcarbonyl.

In certain of these embodiments, R⁸ is C₁-C₆ alkyl or C₆-C₁₀ aryl-C₁-C₆ alkylenyl.

In certain particular embodiments, the compound is selected from the group consisting of:

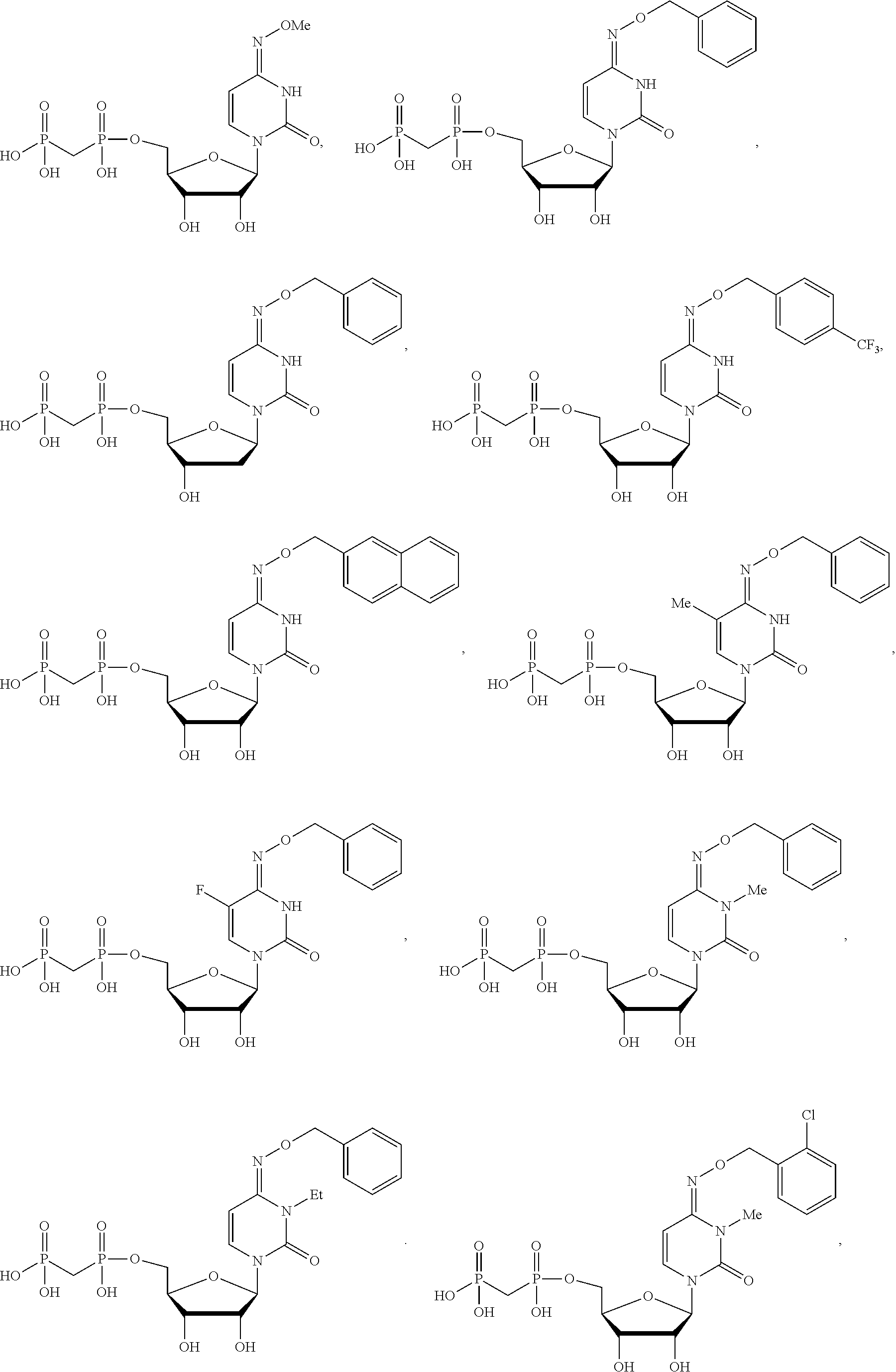

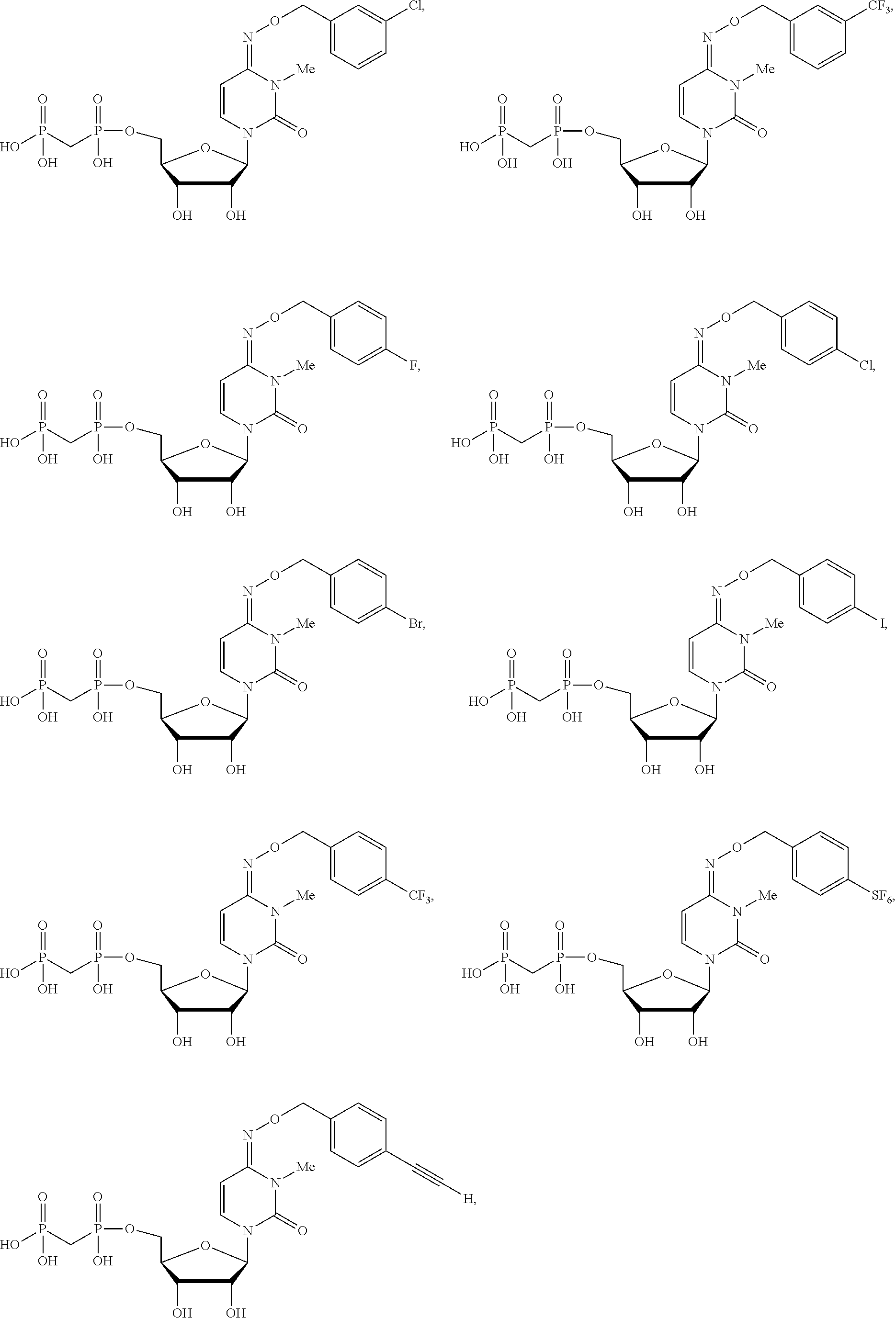

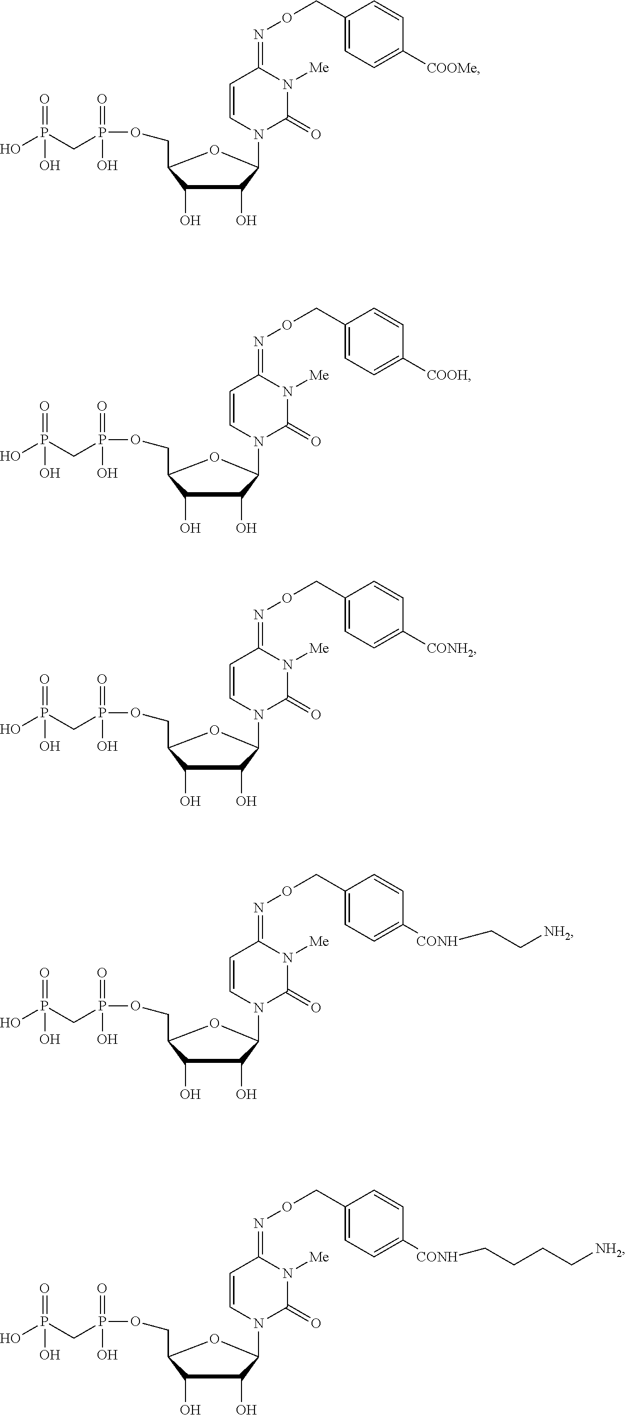

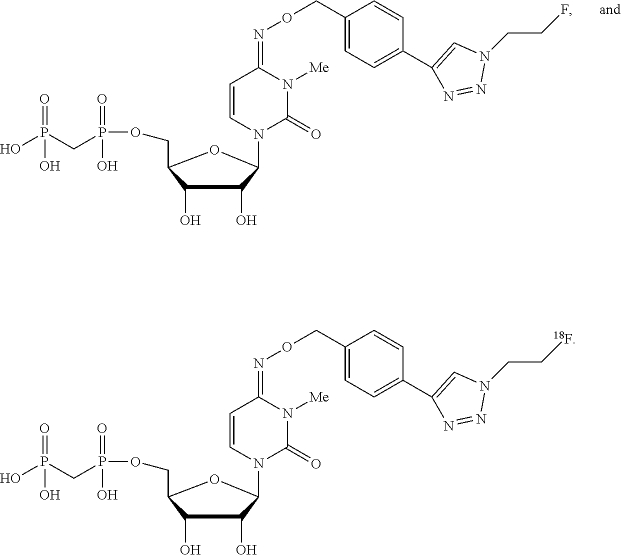

In certain embodiments, A is

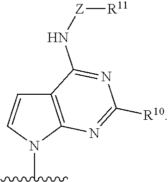

In certain of these embodiments, R¹ is OH and R² is H.

In certain of these embodiments, R¹⁰ is H.

In certain of these embodiments, R⁵ is C₆-C₁₀ aryl.

In certain of these embodiments, Z is CH? or C₄₁₄.

In certain particular embodiments, the compound is selected from the group consisting of:

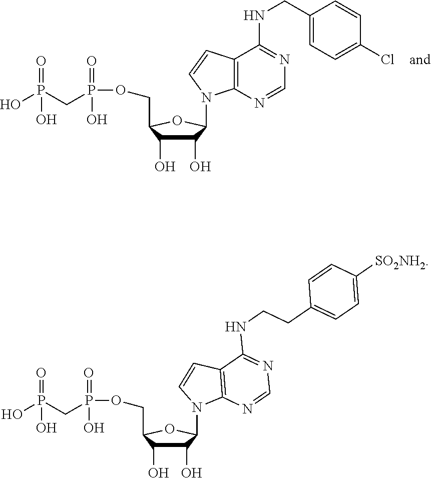

The invention further provides a compound of the formula:

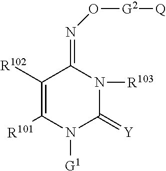

wherein R¹⁰¹ and R¹⁰² are independently H, halo, C₁-C₆ alkyl, C₁-C₆ aryl, NH₇, N₃, C₁-C₆ alkynyl, C₆-C₁₀ aryl-C₁-C₆ alkylenyl, 1-halo-1-vinyl, C₆-C₁₀ heteroaryl-C₁-C₆ alkyl, C₆-C₁₀ heteroaryl or C₆-C₁₀ arylcarbonyl,
R¹⁰³ is H, C₁-C₆ alkyl, C₆-C₁₀ aryl-C₁-C₆ alkyl, C₆-C₁₀ aryl-C₁-C₆ alkylenyl, C₁-C₆ alkylcarbonyl, C₆-C¹⁰ heteroaryl-C₁-C₆ alkyl, C₆-C₁₀ heteroaryl or C₆-C₁₀ arylcarbonyl,
G¹ is

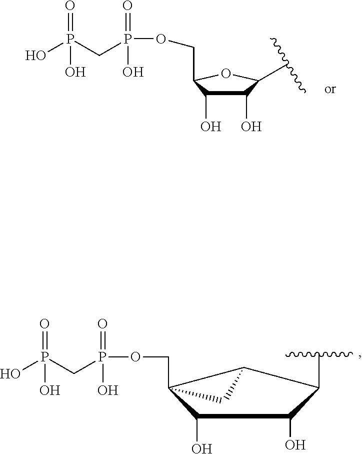

G² is

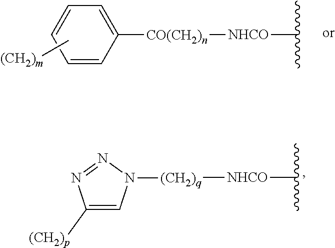

m, n, p, and q are independently integers of from 1 to about 20, and
Q is a fluorophore moiety;
   or a pharmaceutically acceptable salt thereof.

Q can be any suitable fluorophore moiety. In certain embodiments, Q can be a fluorophore moiety selected from the group consisting of FITC, NBD, Dansyl, Squaraine Rotaxane, Bodipy FL, Bodipy TR, Bodipy 630/650-X, Bodipy 650/655-X, Texas Red, Cy5, 1-pyrene, EVOBlue 30, Alexa Fluor 532, Alexa Fluor 488-5, 488-6, or mixture thereof, Tamra, Tamra 5/6-X-SE, Alexa Fluor 488 azide 5 isomer, Alexa Fluor 488 5 isomer, Alexa Fluor 488 5/6 mixed isomers, NIR dye 700, NIR dye 800, Janelia Fluor 549 amide, and Janelia Fluor 646 amide.

In certain embodiments, G¹ is

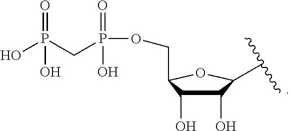

In certain embodiments, G² is

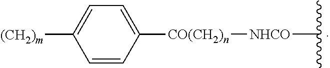

In a particular embodiment, the compound is:

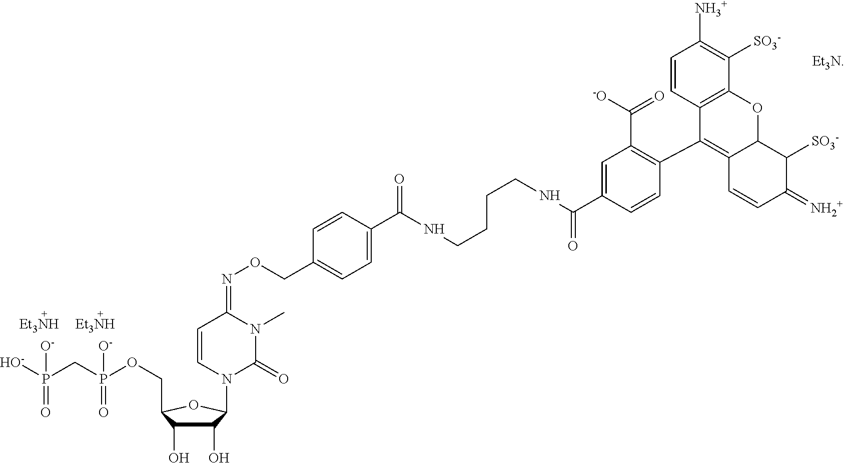

In certain embodiments, G² is

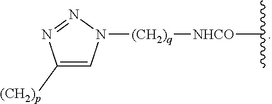

In a particular embodiment, the compound is:

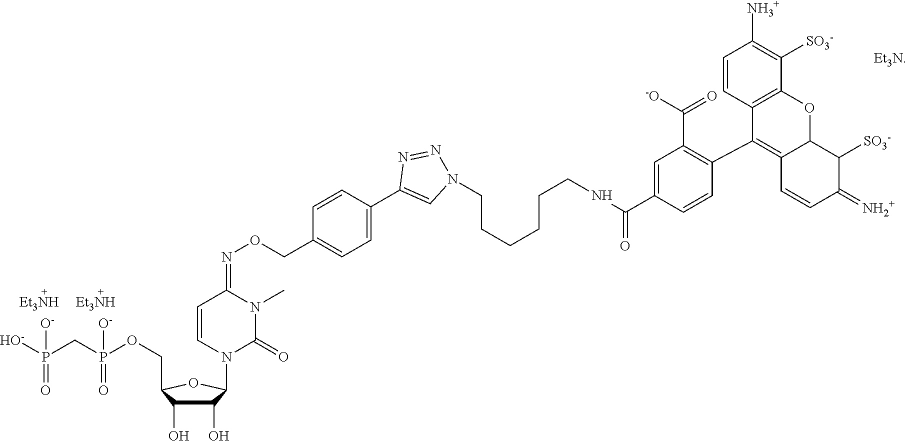

The fluorophore moiety can be linked to G² at any of its suitable reactive site or sites through a direct reaction with a reactive group or through the use of a spacer group. For example, an FITC moiety can be linked at or through a reaction of its isothiocyanate group to an amino group on Y G² A Dansyl moiety can be linked at or through a reaction of its sulfonyl chloride group to an amino group on G². A Bodipy moiety can be linked at or through a reaction of its carboxyl group with an amino group on G². Alexa Fluor 532 can be linked at or through a reaction of its maleimide activated benzoate group to an amino or hydroxyl group on G². EVOBlue 30 can be linked at or through a reaction of its carboxyl group with an amino group on G². Alternatively, a spacer group can be employed to link the fluorophore moiety to G². Examples of spacer groups include aminoalkyl carboxyl groups, amino alkoxy carboxyl groups, amino polyalkoxy carboxyl groups, dicarboxylic acid groups, polyamines, diamines, and the like.

The present invention further provides a diagnostic composition comprising a compound or salt as described above and a pharmaceutically acceptable carrier.

The phrase “pharmaceutically acceptable salt” is intended to include nontoxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, nonaqueous media such as ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, p. 1445, and Journal of Pharmaceutical Science, 66, 2-19 (1977).

Suitable bases include inorganic bases such as alkali and alkaline earth metal bases, e.g., those containing metallic cations such as sodium, potassium, magnesium, calcium and the like. Non-limiting examples of suitable bases include sodium hydroxide, potassium hydroxide, sodium carbonate, and potassium carbonate. Suitable acids include inorganic acids such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, maleic acid, tartaric acid, fatty acids, long chain fatty acids, and the like. Preferred pharmaceutically acceptable salts of inventive compounds having an acidic moiety include sodium and potassium salts. Preferred pharmaceutically acceptable salts of inventive compounds having a basic moiety (e.g., a dimethylaminoalkyl group) include hydrochloride and hydrobromide salts. The compounds of the present invention containing an acidic or basic moiety are useful in the form of the free base or acid or in the form of a pharmaceutically acceptable salt thereof.

It should be recognized that the particular counterion forming a part of any salt of this invention is usually not of a critical nature, so long as the salt as a whole is pharmacologically acceptable and as long as the counterion does not contribute undesired qualities to the salt as a whole.

It is further understood that the above compounds and salts may form solvates, or exist in a substantially uncomplexed form, such as the anhydrous form. As used herein, the term “solvate” refers to a molecular complex wherein the solvent molecule, such as the crystallizing solvent, is incorporated into the crystal lattice. When the solvent incorporated in the solvate is water, the molecular complex is called a hydrate. Pharmaceutically acceptable solvates include hydrates, alcoholates such as methanolates and ethanolates, acetonitrilates and the like. These compounds can also exist in polymorphic forms.

Chemistry

There are several commonly used multi-step methods for the preparation of nucleoside-5′-O-[(phosphonomethyl)phosphonic acid] derivatives, i.e., either reacting the protected nucleoside with activated bisphosphonate or utilizing methylene diphosphonic acid and coupling reagents (4-6). However, despite the use of protecting groups these synthetic strategies suffer overall from very low yields. Previously, it was demonstrated that phosphonylation reactions of unprotected nucleosides using methylenebis(phosphonic dichloride) in trimethyl phosphate provided the nucleoside-5′-O-[(phosphonomethyl)phosphonic acids] as the main products under optimized conditions (12). Furthermore, reacting unprotected nucleosides with 1.5 eq. methylene diphosphonic acid and 3 eq. dicyclohexylcarbodiimide (DCC) in dimethylformamide (DMF) led as well to the formation of nucleoside-5′-O-[(phosphonomethyl)phosphonic acids] as the main products. Therefore, solely unprotected nucleosides were employed as starting materials in the preparation of compounds of the invention. When additional phosphonylation occurred at the 2′- and/or 3′-position, these side products were easily separated from the desired 5′-substituted product through a combination of ion exchange and reverse phase C₁₈ chromatography. The SARs of the purine scaffold in AOPCP (adenosine-5′-O-[(phosphonomethyl)phosphonic acid) derivatives was extensively explored in a previous study, leading to CD73 inhibitors with potency in low nanomolar range (12). However, the SARs of the ribose moiety of AOPCP derivatives had not been explored. Therefore, 2′-deoxy (2a), 2′-amino-2′-deoxy (2b), and 3′-deoxy (2d) AOPCP derivatives were prepared by reacting the respective nucleoside with methylene diphosphonic acid in the presence of DCC in DMF (Scheme 1). In case of 2b, the reaction proceeded very slowly, and the 3′-phosphonate side product (2c) was formed to an equal extent (0.5% yield). Compound 2c was isolated, and its structure was unequivocally determined using ¹H-¹H-COSY NMR spectra. To address ring variations of the adenine moiety, the 1-deaza (2e), 3-deaza (2f), and 7-deaza (2g) AOPCP derivatives were prepared by reaction of unprotected nucleosides with methylenebis(phosphonic dichloride) in trimethyl phosphate to increase yields (Scheme 1).

Focus was turned to pyrimidine-derived 5′-O-[(phosphonomethyl)phosphonic acids, e.g., UOPCP (4a), prepared by phosphonylation of uridine (3a). Furthermore, N³-substituted nucleosides 3b-e were prepared via nucleophilic substitution using 3a, and their subsequent phosphonylation afforded compounds 4b-e. Substitution of the uracil 5-position was explored with methyl 4f-h and halogen 4l-o derivatives. The reaction of 5-ethynyl-uridine with methylenebis(phosphonic dichloride) led, besides the desired 5-ethynyl-uridine derivative 4i, to the formation of 5-(1-chlorovinyl)uridine-(4j) and 5-(1-chlorovinyl)-3-methyluridine-5′-O-[(phosphonomethyl)phosphonic acid] (4k) through the addition of HCl to the alkyne bond. In order to explore the variations of the 2′-position at the ribose moiety 2′-deoxy (4p), 2′-amino-2′-deoxy (4q), 2′-azido-2′-deoxy (4r), and 2′-fluoro-2′-deoxy (4s) derivatives were prepared. The role of the stereochemistry at the 2′-position was addressed through the synthesis of 2′-ara-fluoro-2′-deoxyuridine-(4t) and 1-(β-D-arabinofuranosyl)-uridine-5′-O-[(phosphonomethyl)phosphonic acid (4u). Synthesis of 6-azauridine derivative (4v) allowed the introduction of an H-bond acceptor at the 6-position. In addition to the methylene group, the linker between the two phosphonate groups was extended to an ethylene moiety by reacting ethylene diphosphonic acid with the respective nucleosides 3a, 3f to afford uridine-(4w) and 5-methyluridine-5′-O-[(phosphonoethyl)phosphonic acid] (4x). 2-Thiouridine derivative 4y was prepared following a previously published procedure (7).

Cytidine derivatives varying at the 2′-position (7a and 7b), as well as at the 5-position (7c-e), were prepared as depicted in Scheme 2. The N⁴-benzoyl COPCP derivative 7f displayed rapid decomposition in aqueous solution to UOPCP (4a). In order to introduce bulky aromatic substituents at the 4-position of the cytosine moiety without the inherent instability issues as seen with N⁴-benzoyl COPCP derivative 7f, focus was turned towards alkoxyimino derivatives 9a-i. The required nucleosides were prepared by either reacting cytidine (6a), 2′-deoxycytidine (6b), 5-fluorocytidine (6d) or 5-methylcytidine (6e) with respective alkoxyamino derivatives in pyridine⁸ followed by phosphonylation reaction to afford compounds 9a-f, or by reacting cytidine (6a) with benzyloxyamine, followed by nucleophilic substitution at the 3-position and subsequent 5′-O-phosphonylation of the nucleosides to afford compounds 9h and 9i (Scheme 2). Furthermore, 3-deazauridine-5′-α,β-methylene-diphosphate (10), (S)-methanocarba-5′-α,β-methylene-diphosphate (11) and the ethyl ester of 2-thio-UDP (12) (7) were prepared to extend the exploration of SARs of the nucleotide analogs as CD73 inhibitors. All phosphonate-substituted nucleotides were purified to homogeneity by ion exchange chromatography followed by reverse phase C₁₈ HPLC.

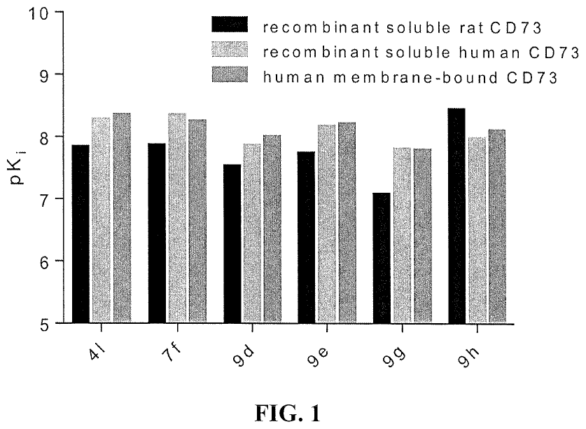

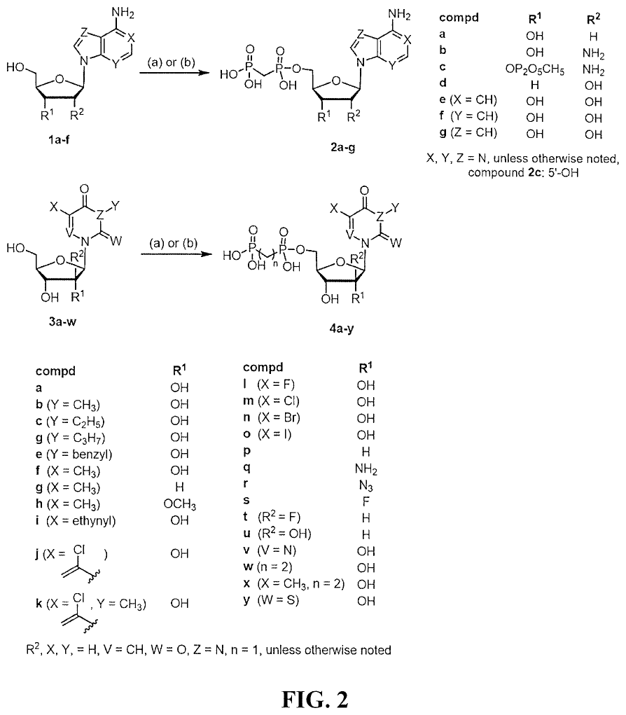

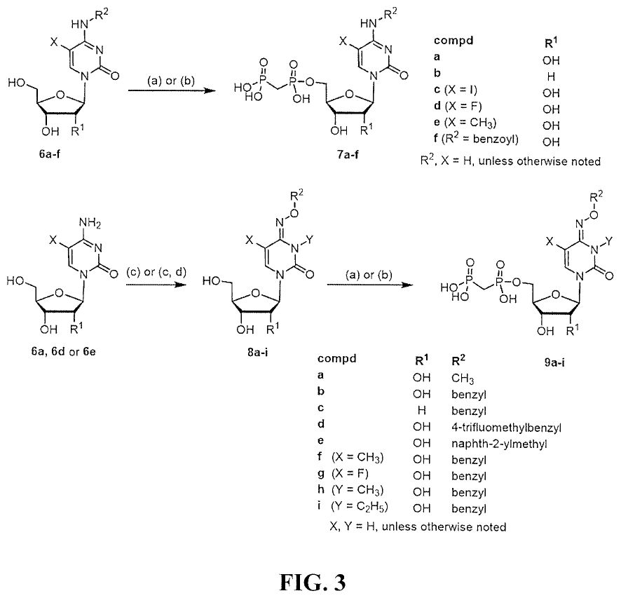

Syntheses of compounds in accordance with embodiments of the invention are shown in FIGS. 1 and 2.

Compound 101-104 were prepared as previously reported.

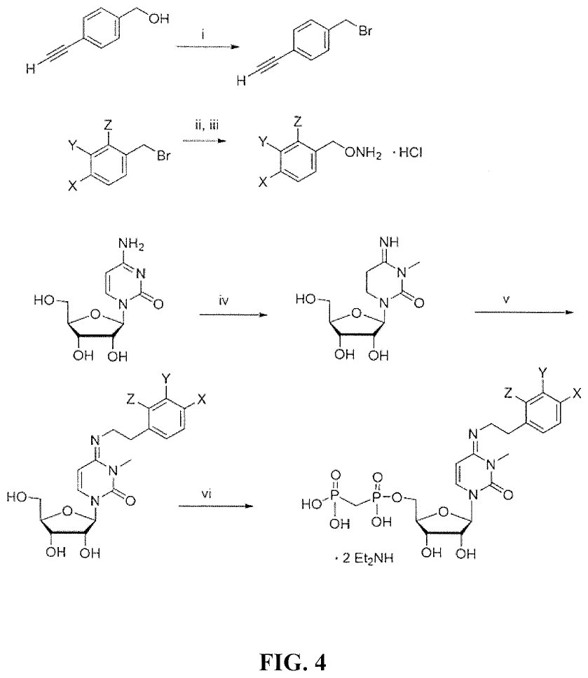

As shown in FIG. 4, synthesis of compounds 105-115 required the preparation of various substituted benzyloxyamines (125-35), which were produced following a literature procedure, starting from the corresponding substituted benzyl bromides. Similar methods were used to prepare phenylethyl and phenylpropyl homologues (structures not shown) of the benzyl derivatives. In the case of compound 134, as the corresponding benzyl bromide was not commercially available, the latter was synthesized reacting the 4-ethynylbenzyl alcohol with phosphorus (III) tribromide.

The 3-methylcytidine (136) was prepared using methyl iodide in dimethylacetamide (Dmac) without the necessity of any base catalyst addition. On the contrary, for the synthesis of intermediates 158, K₇CO₃ was required to obtain the desired N-3 methylated product, as there was no free 4-amino group on the cytosine moiety able to stabilize the alkylation transition state.

Reaction of 136 with the desired substituted benzyloxyamine (25-35) in pyridine at 80° C., gave the desired intermediates 137-147 in good yield. 5′-O-phosphorylation of the latter was performed using methylenebis(phosphonic dichloride) in trimethyl phosphate obtaining the desired final compound 5-15 in acceptable yields.

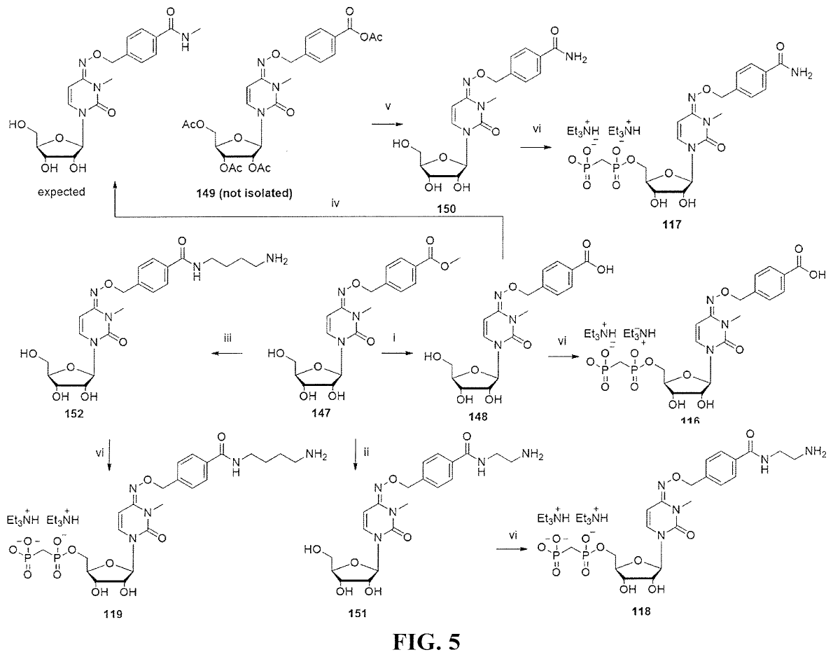

FIG. 5 shows a synthetic route for the preparation of derivatives 116-119. Hydrolysis of the methyl ester moiety of compound 47 using sodium hydroxide furnished a p-carboxybenzyl nucleoside intermediate 148 that was 5′-phosphorylated as previously indicated to obtain 116.

Starting from compound 148, it was planned to prepare its methylamide derivative using HATU and DIPEA as coupling agents between the carboxylic acid moiety and the methylamine. LC-MS of the so obtained product indicated the formation of an acetylated derivative (149, probably because of the presence of acetic acid traces used during the previous step) that was not fully characterized but directly reacted with ammonia to try to remove the acetyl groups and recover the starting compound 148. Instead, the obtained derivative was 150, that was used to prepare compound 117.

The two amine congeners 151 and 152 were synthesized by amination of 147 with ethylenediamine or 1,4-diaminobutene, respectively. The so obtained crude amides were directly used for 5′-O-phosphorylation without further purification, but using an increased amount of methylenebis(phosphonic dichloride) to be sure to have a full conversion of starting compounds to the desired products 118 and 119.

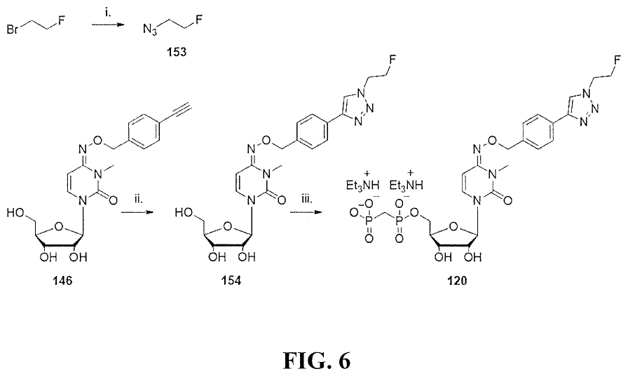

1-Fluoro-2-azido-ethane (153, FIG. 6) was prepared following a literature procedure. A Click-reaction between the latter and 146 gave the 1,2,3-triazole analog 154 that was used to obtain the desired product 120.

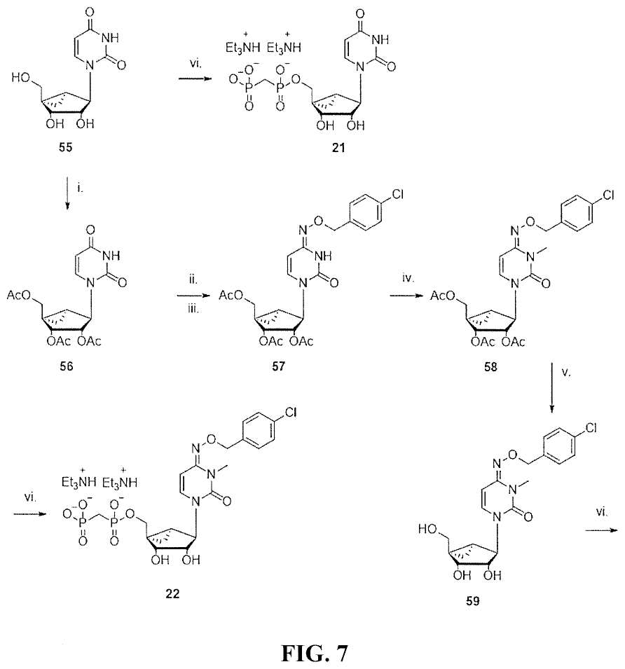

A methanocarba ([3.1.0]bicyclohexane) substitution of the ribose ring in nucleosides and nucleotides allows the pre-establishment of a conformation, either North (N) or South (S) depending on the position of cyclopropane ring fusion, tailored for the target protein interaction. (N)-Methanocarba analogs 121 and 122 of compound 155 were prepared (FIG. 7) by adapting reported procedures. Sugar protection by acetylation was performed using the standard method with acetic anhydride to obtain intermediate 156. Next, the 4-ketone moiety was first activated through reaction with 2,4,6-triisopropylbenzenesulfonyl chloride (TPSCl), followed by nucleophilic displacement by 4-C₁-benzyloxyamine (129) to obtain compound 157. N³ methylation of the latter was performed as described above obtaining 158 and then, removal of the acetyl groups was achieved using methanolic ammonia solution to afford intermediate 159 in good yield. The two (N)-methanocarba-5′-O-α,β-methylenediphosphates 121 and 122 were prepared starting from the corresponding precursors 155 and 159 using a method already described.

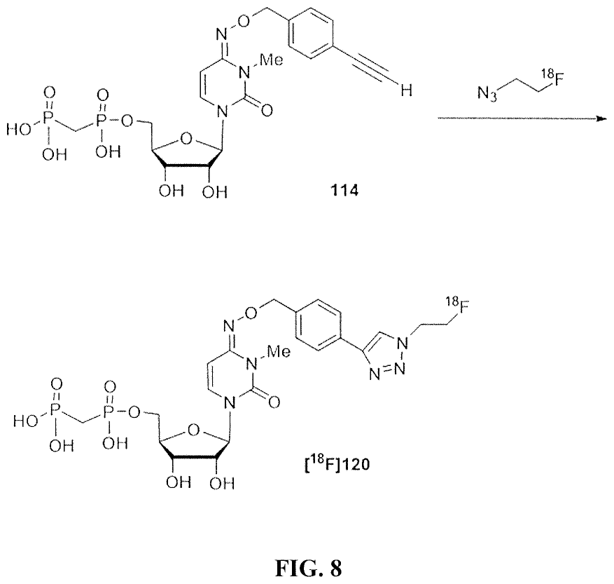

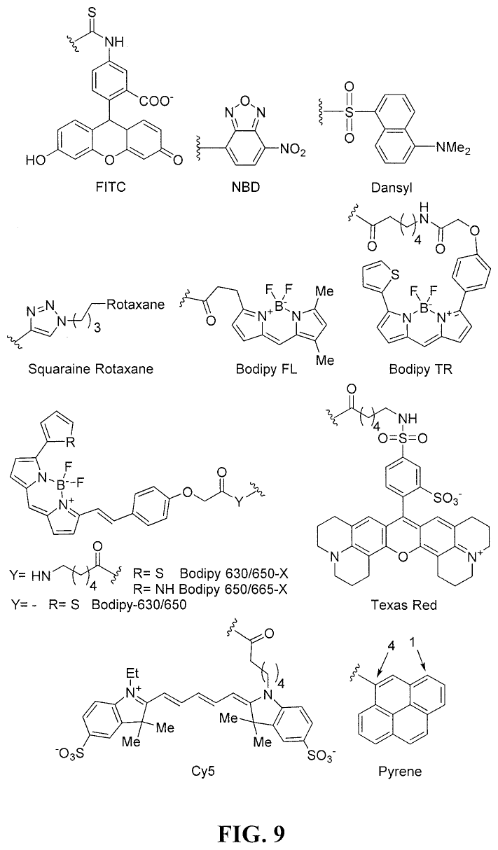

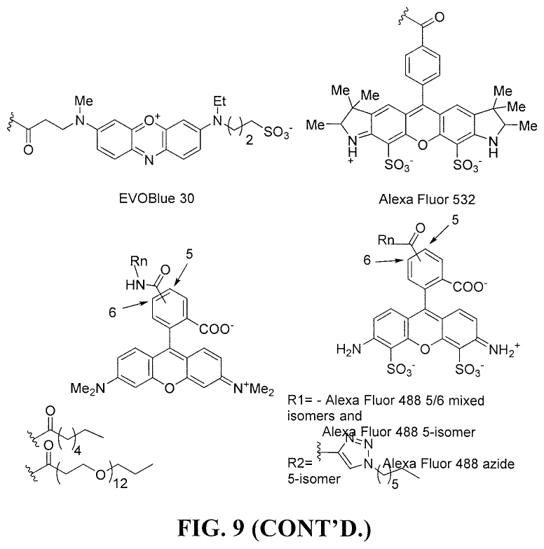

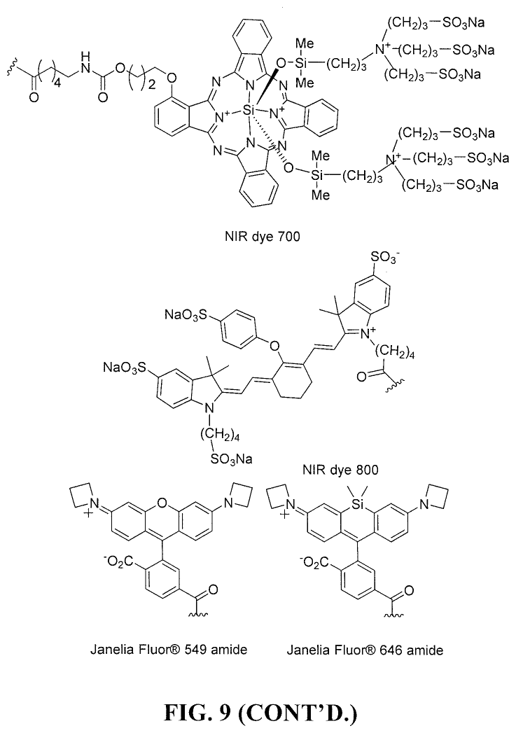

FIG. 8 shows the introduction of a ¹⁸F to compound 20 via a Click reaction on a α,β-methylenediphosphate derivative, 114. When using this reaction to produce unlabeled 120 stoichiometrically, the reaction time may be as long as 12 hours, but the radioligand [¹⁸F]20 synthesis is on a time scale consistent with the ˜2 hr half-life of the radioisotope.

Pharmacological Evaluation

The potency of the compounds to inhibit CD73 was determined by a radiometric CD73 assay using [2,8-³H]AMP as a substrate and recombinant soluble rat CD73 (33). After the enzymatic reaction, the substrate was separated from its product [2,8-³H]adenosine by precipitation with lanthanum chloride followed by filtration through glass fiber filters (34). For compounds that inhibited CD73 activity by more than 50% at an initial screening concentration of 1 μM, full concentration-response curves were determined in at least three separate experiments performed in duplicates using 10 different concentrations of inhibitor. Kᵢ values were calculated from the obtained IC₅₀ values using the Cheng-Prusoff equation (6,7). Results are summarized in Tables 1-3. Rat CD73 is similar to the human variety (87% sequence identity, BLAST algorithm (35,36) and the active sites only differ in a single amino acid (Phe in the human enzyme is replaced by Tyr in rat) (34). Previous studies had shown that the potency of competitive inhibitors targeting rat CD73 showed comparable or higher potency for human CD73 (12). Nevertheless, the most potent compounds (4l, 7f, 9d, 9e, 9g and 9h) were analyzed using recombinant soluble human CD73. Furthermore, to study the inhibitors in a less artificial environment, they were investigated on membrane-anchored CD73 using membrane preparations derived from the triple-negative breast cancer (TNBC) cell line MDA-MB-231. These cells overexpress CD73, serve as in vitro model for TNBC and had previously been used for the development of therapeutic antibodies against CD73 (31, 37, 38, 39, 40).

The present invention further provides a pharmaceutical composition comprising a compound as described above and a pharmaceutically acceptable carrier. The present invention provides a pharmaceutical composition comprising a pharmaceutically acceptable carrier and an effective amount, e.g., a therapeutically effective amount, including a prophylactically effective amount, of one or more of the aforesaid compounds, or salts thereof, of the present invention.

The pharmaceutically acceptable carrier can be any of those conventionally used and is limited only by chemico-physical considerations, such as solubility and lack of reactivity with the compound, and by the route of administration. It will be appreciated by one of skill in the art that, in addition to the following described pharmaceutical compositions; the compounds of the present invention can be formulated as inclusion complexes, such as cyclodextrin inclusion complexes, or liposomes.

The pharmaceutically acceptable carriers described herein, for example, vehicles, adjuvants, excipients, or diluents, are well known to those who are skilled in the art and are readily available to the public. It is preferred that the pharmaceutically acceptable carrier be one which is chemically inert to the active compounds and one which has no detrimental side effects or toxicity under the conditions of use.

The choice of carrier will be determined in part by the particular active agent, as well as by the particular method used to administer the composition. Accordingly, there is a wide variety of suitable formulations of the pharmaceutical composition of the present invention. The following formulations for oral, aerosol, parenteral, subcutaneous, intravenous, intraarterial, intramuscular, interperitoneal, intrathecal, rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

The compounds of the present invention, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compounds of the present invention may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topical formulations, including those that are useful for transdermal drug release, are well-known to those of skill in the art and are suitable in the context of the invention for application to skin. Topically applied compositions are generally in the form of liquids, creams, pastes, lotions and gels. Topical administration includes application to the oral mucosa, which includes the oral cavity, oral epithelium, palate, gingival, and the nasal mucosa. In some embodiments, the composition contains at least one active component and a suitable vehicle or carrier. It may also contain other components, such as an anti-irritant. The carrier can be a liquid, solid or semi-solid. In embodiments, the composition is an aqueous solution. Alternatively, the composition can be a dispersion, emulsion, gel, lotion or cream vehicle for the various components. In one embodiment, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions can be produced as solids, such as powders or granules. The solids can be applied directly or dissolved in water or a biocompatible solvent prior to use to form a solution that is substantially neutral or that has been rendered substantially neutral and that can then be applied to the target site. In embodiments of the invention, the vehicle for topical application to the skin can include water, buffered solutions, various alcohols, glycols such as glycerin, lipid materials such as fatty acids, mineral oils, phosphoglycerides, collagen, gelatin and silicone based materials.

Additionally, the compounds of the present invention may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

In an embodiment, the invention provides a method of inhibiting ecto-5′-nucleotidase in a mammal in need thereof, comprising administering to the mammal an effective amount of a compound or salt of the invention. In a preferred embodiment, the ecto-5′-nucleotidase is associated with a cancer cell.

In another embodiment, the invention provides a method of inhibiting suppression of an antitumor immune response in a mammal in need thereof, comprising administering to the mammal an effective amount of a compound or salt of the invention. In a preferred embodiment, the antitumor response is mediated via activation of adenosine 2A (A2A) receptors.

In certain of these embodiments, the mammal is afflicted with lung cancer, prostate cancer, triple-negative breast cancer, melanoma, leukemia, or thyroid cancer.

In a further embodiment, the invention provides a method of inhibiting tumor growth of a cancerous tumor in a mammal in need thereof, comprising administering to the mammal an effective amount of a compound or salt of the invention. In certain embodiments, the tumor is associated with lung cancer, prostate cancer, triple-negative breast cancer, or thyroid cancer.

In yet another embodiment, the invention provides a method of inhibiting metastasis of cancer in a mammal afflicted with cancer, comprising administering to the mammal an effective amount of a compound or salt of the invention.

In preferred embodiments, the tumor is associated with lung cancer, prostate cancer, triple-negative breast cancer, or thyroid cancer.

In another embodiment, the invention provides a method of synergistically enhancing a response of a mammal afflicted with cancer undergoing treatment with an immunotherapeutic anti-cancer agent, comprising co-administering to the mammal an effective amount of a compound or salt of the invention.

In certain of these embodiments, the tumor is associated with lung cancer, prostate cancer, triple-negative breast cancer, or thyroid cancer.

In certain preferred embodiments, the immunotherapeutic anti-cancer agent is selected from the group consisting of pembrolizumab, nivolumab, atezolizumab, durvalumab, and ipilimumab.

In a further embodiment, the invention provides a method of potentiating an activity of an inhibitor of nicotinamide phosphoribosyltransferase in a mammal undergoing treatment of a mammal with the inhibitor, comprising administering to the mammal an effective amount of a compound or salt of the invention.

In a particular embodiment, the inhibitor of nicotinamide phosphoribosyltransferase is FK866.

In yet a further embodiment, the invention provides a method of treating preeclampsia in in a mammal in need thereof, comprising administering to the mammal an effective amount of a compound or salt of the invention.

In a further embodiment, the invention provides a method of treating cancer in a mammal comprising administering to the animal an effective amount of a compound or salt of the invention, wherein the cancer is lung cancer, prostate cancer, triple-negative breast cancer, melanoma, leukemia, or thyroid cancer.

As used herein, the term “treat” does not necessarily imply complete elimination of a cancer. Rather, there are varying degrees of treatment of which one of ordinary skill in the art recognizes as having a benefit or therapeutic effect. In this respect, the cancer can be treated to any extent through the present inventive method. For example, at least 10% (e.g., at least 20%, 30%, or 40%) of the growth of a cancerous tumor desirably is inhibited upon administration of a compound described herein. Preferably, at least 50% (e.g., at least 60%, 70%, or 80%) of the growth of a cancerous tumor is inhibited upon administration of a compound described herein. More preferably, at least 90% (e.g., at least 95%, 99%, or 100%) of the growth of a cancerous tumor is inhibited upon administration of a compound described herein. In addition or alternatively, the inventive method may be used to inhibit metastasis of a cancer.

In an embodiment, the inventive compounds are useful in imaging of a mammal in need thereof. In certain embodiments, the imaging is performed using positron emission tomography (PET). In these embodiments, a compound of the invention, preferably a compound labeled with ¹⁸F, is administered to the mammal, and the mammal is imaged.

The present invention further provides a diagnostic method for determining a treatment of a patient for a possible antagonist of the ecto-5′-nucleotidase, the treatment comprising: (a) administering a compound or salt as described above; (b) obtaining a biological sample from the patient; (c) determining the level of expression of ecto-5′-nucleotidase in the biological sample; (d) comparing the level of expression of the ecto-5′-nucleotidase to that of a normal population; and (e) if the patient's level of expression is higher than that of the normal population, determining a treatment regimen comprising administering an antagonist of the ecto-5′-nucleotidase whose expression was higher in the patient than that of the normal population.

### Illustrative Examples of Embodiments

The invention contains at least the following embodiments:
1. A compound of formula (I):

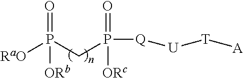

wherein Q is O, S, CH₂, or NH;
U is O, CH₂, C₂H₄, NH or S;
n is an integer of from 1 to 6;
R^{a}, R^{b} and R^{c} are independently H, C₁-C₆ alkyl, C₆-C₁₀ aryl, C₁-C₆ alkylcarbonyl, C₆-C₁₀ heteroaryl-C₁-C₆ alkyl, C₆-C₁₀ heteroaryl, or C₆-C₁₀ arylcarbonyl;

T is

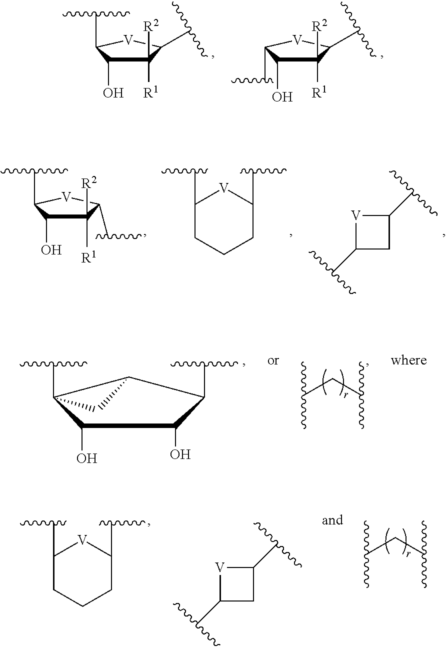

are optionally substituted with 1, 2, or 3 hydroxyl groups and r is an integer from 2 to about 6,
V is O, S, CH₂ or NH;

A is

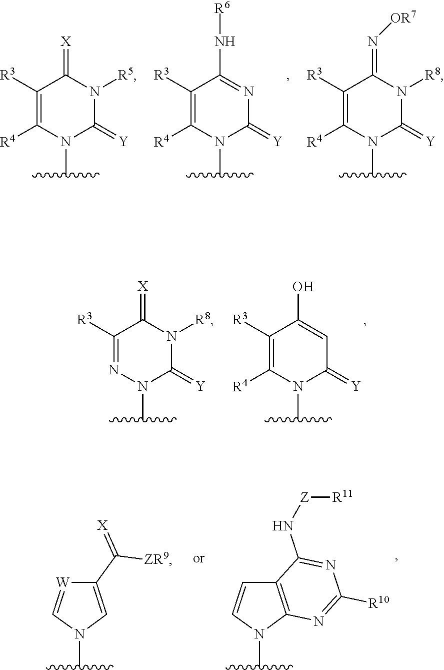

wherein X, Y, and Z are independently O, S, NH or C₁-C₆ alkylenyl, and W is independently N or CH,

R¹ and R² are independently H, OH, SH, C₁-C₆ alkoxy, C₁-C₆ thioalkoxy, NH₂, C₁-C₆ alkylamino, N₃, or halo;

R³, R⁴ and R¹⁰ are independently H, halo, C₁-C₆ alkyl, C₁-C₆ aryl, NH₂, N₃, C₁-C₆ alkynyl, C₆-C₁₀ aryl-C₁-C₆ alkylenyl, 1-halo-1-vinyl, C₆-C₁₀ heteroaryl-C₁-C₆ alkyl, C₆-C₁₀ heteroaryl or C₆-C₁₀ arylcarbonyl,

R⁵ to R⁸ and R^{1′} are independently H, C₁-C₆ alkyl, C₆-C₁₀ aryl-C₁-C₆ alkyl, C₆-C₁₀ aryl-C₁-C₆ alkylenyl, C₁-C₆ alkylcarbonyl, C₆-C₁₀ heteroaryl-C₁-C₆ alkyl, C₆-C₁₀ heteroaryl or C₆-C₁₀ arylcarbonyl,

R⁹ is C₁-C₆ alkyl, C₆-C₁₀ aryl-C₁-C₆ alkylenyl, C₁-C₆ alkylcarbonyl, C₆-C₁₀ heteroaryl-C₁-C₆ alkyl, C₆-C₁₀ heteroaryl or C₆-C₁₀ arylcarbonyl,
wherein aryl at each occurrence is optionally substituted with one or more substituents selected from the group consisting of C₁-C₆ alkyl, C₂-C₆ alkynyl, halo, trifluoromethyl, OH, SH, NH₂, SO₂NH₂, C₁-C₆ hydroxyalkyl, C₁-C₆ alkoxy, sulfonyloxy, C₁-C₆ carboxyalkyl, carboxy, carboxamide, C₁-C₆ sulfonyloxyalkyl, arylcarbonyl, CONH(CH)ₚNH₂,

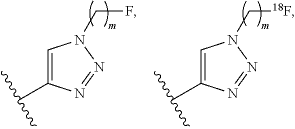

and any combination thereof, wherein heteroaryl is optionally substituted with one or more substituents selected from C₁-C₆ alkyl, halo, trifluoromethyl, C₁-C₆ hydroxyalkyl, C₁-C₆ alkoxy, sulfonyloxy, C₁-C₆ carboxyalkyl, C₁-C₆ sulfonyloxyalkyl, arylcarbonyl,

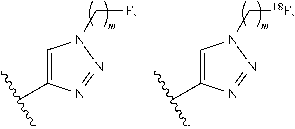

and any combination thereof,
wherein m is an integer of from 2 to about 10,
wherein p is an integer of from 2 to about 10,
with the proviso that when R^{a}, R^{b}, and R^{c} are all H, n is 1, Q is O, U is CH₂, T is

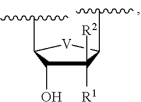

V is O, R¹ is OH, R² is H, and A is

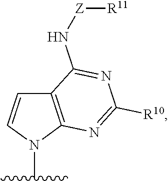

R⁵ is not H, methyl, ethyl, or benzyl and R¹⁰ is not H.
or a pharmaceutically acceptable salt thereof.
2. The compound or salt of embodiment 1, wherein Q is O, U is CH₂, and T is

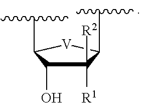

3. The compound or salt of embodiment 1 or 2, wherein A is

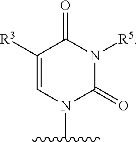

4. The compound or salt of embodiment 3, wherein R¹ is H, OH, NH₂, N₃, C₁-C₆ alkoxy or halo and R² is H or halo.
5. The compound or salt of embodiment 3 or 4, wherein R³ is H, halo, methyl, ethynyl, or 1-chloro-1-vinyl.
6. The compound or salt of any one of embodiments 3-5, wherein R⁵ is H, methyl, ethyl, propyl or benzyl.
7. The compound or salt of any one of embodiments 3-6, wherein the compound is selected from the group consisting of:

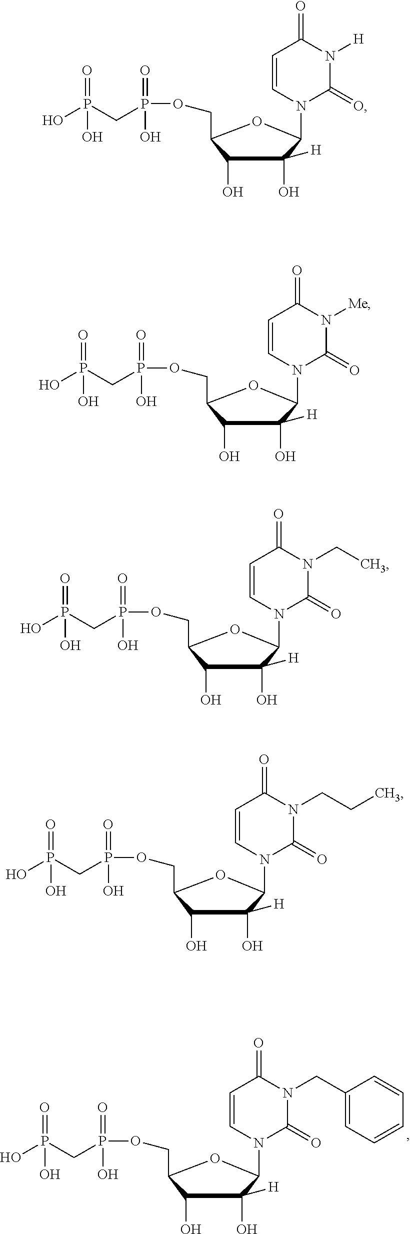

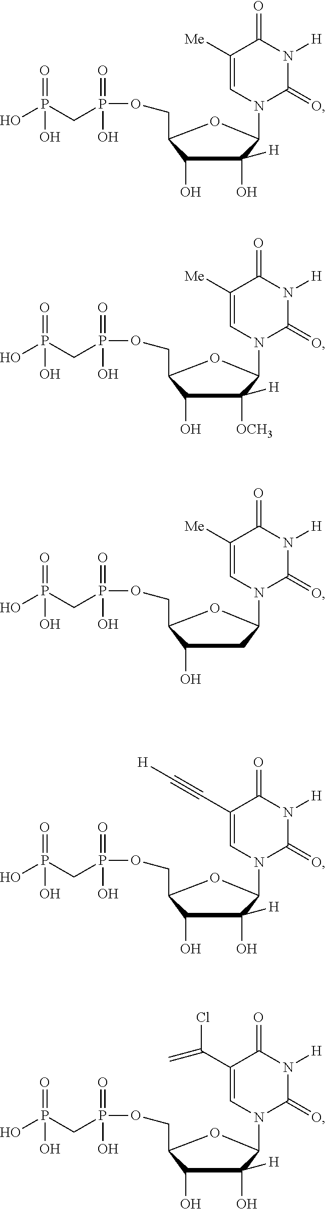

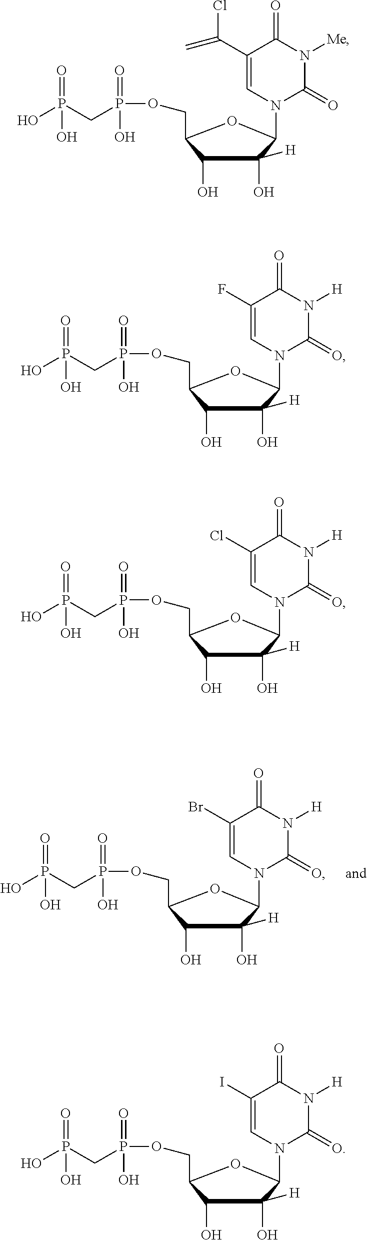

8. The compound or salt of embodiment 3, wherein R¹ is H and R² is F or OH.
9. The compound or salt of embodiment 8, wherein the compound is selected from the group consisting of:

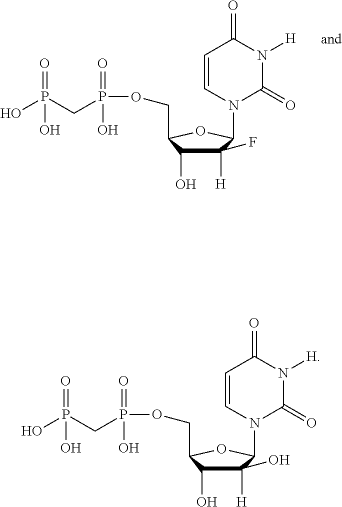

10. The compound or salt of embodiment 1 or 2, wherein T is

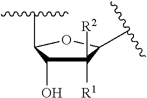

and A is

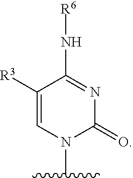

11. The compound or salt of embodiment 10, wherein R¹ is OH or H, and R² is H.
12. The compound or salt of embodiment 10 or 11, wherein R³ is H, halo or C₁-C₆ alkyl.
13. The compound or salt of any one of embodiments 10-12, wherein R⁶ is H, C₁-C₆ alkyl, C₁-C₆-alkylcarbonyl or C₆-C₁₀ arylcarbonyl.
14. The compound or salt of any one of embodiments 10-13, wherein the compound is selected from the group consisting of:

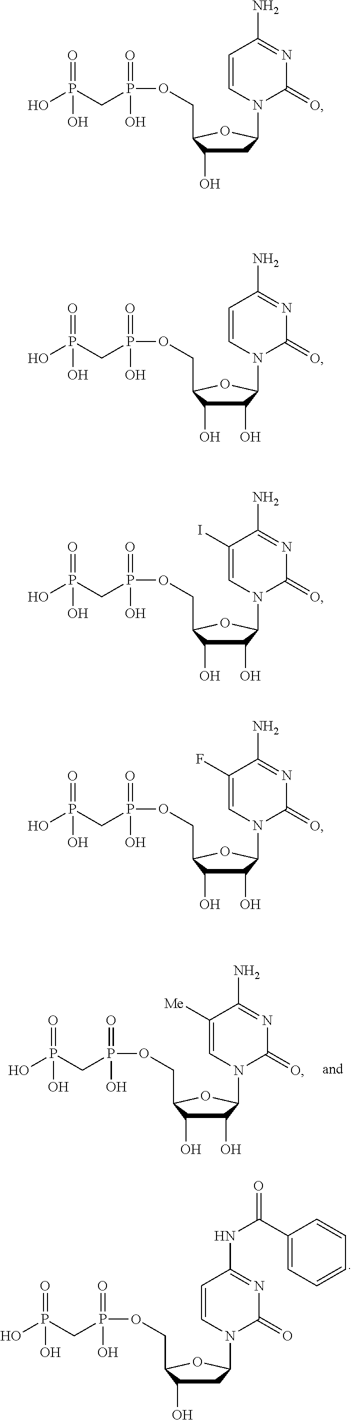

15. The compound or salt of embodiment 1 or 2, wherein T is

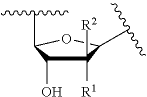

and A is

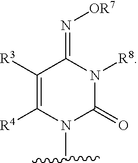

16. The compound or salt of embodiment 15, wherein R¹ is H or OH and R² is H.
17. The compound or salt of embodiment embodiments 15 or 16, wherein R³ is H, halo or C₁-C₆ alkyl.
18. The compound or salt of any one of embodiments 15-17, wherein R⁷ is H, C₁-C₆ alkyl, C₆-C₁₀ aryl-C₁-C₆ alkyl, C₆-C₁₀ aryl-C₁-C₆ alkylenyl, C₁-C₆ alkylcarbonyl, C₆-C₁₀ heteroaryl-C₁-C₆ alkyl, C₆-C₁₀ heteroaryl or C₆-C₁₀ arylcarbonyl.
19. The compound or salt of any one of embodiments 15-18, wherein R⁸ is C₁-C₆ alkyl or C₆-C₁₀ aryl-C₁-C₆ alkylenyl.
20. The compound or salt of any one of embodiments 15-19, wherein the compound is selected from the group consisting of:

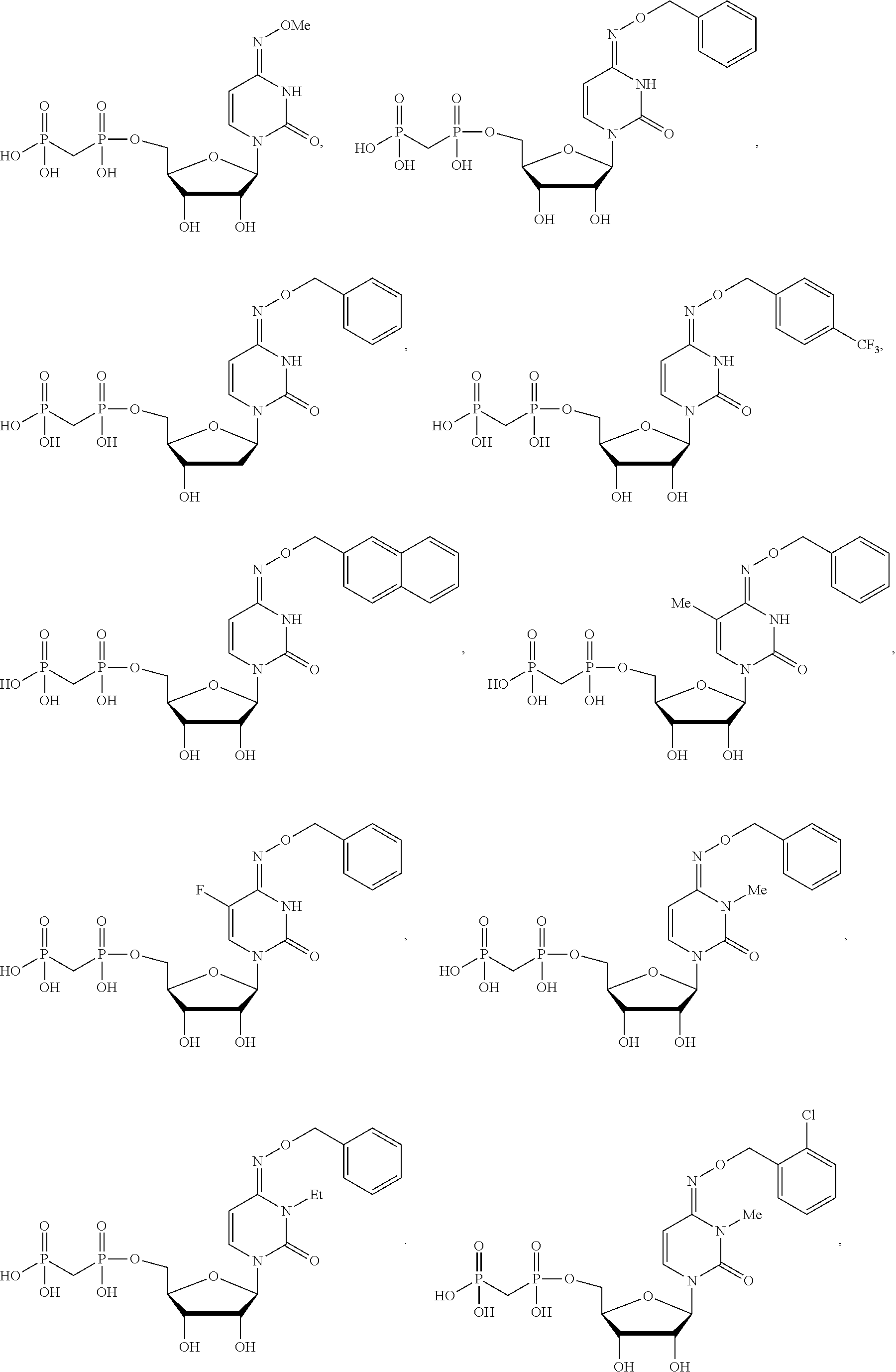

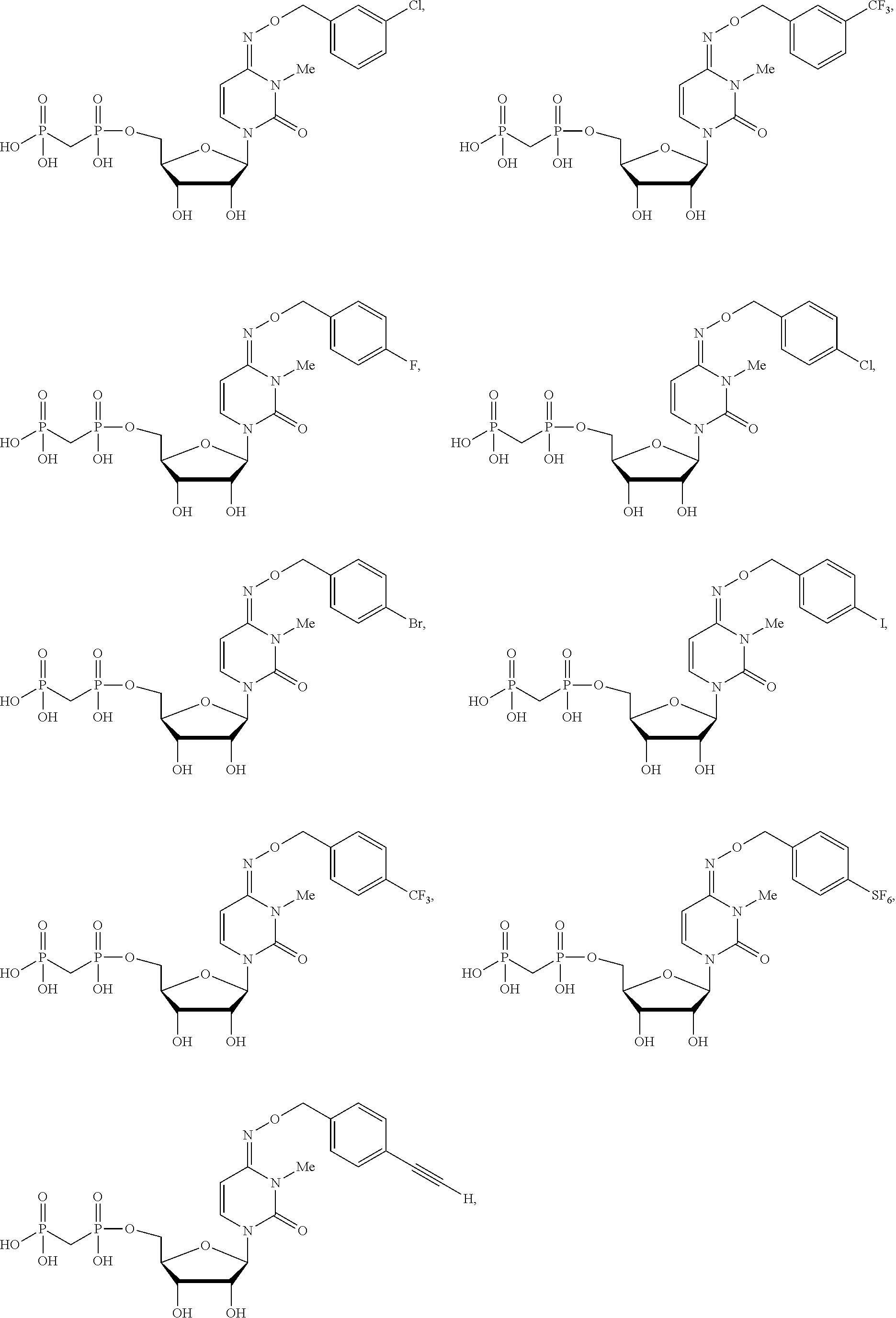

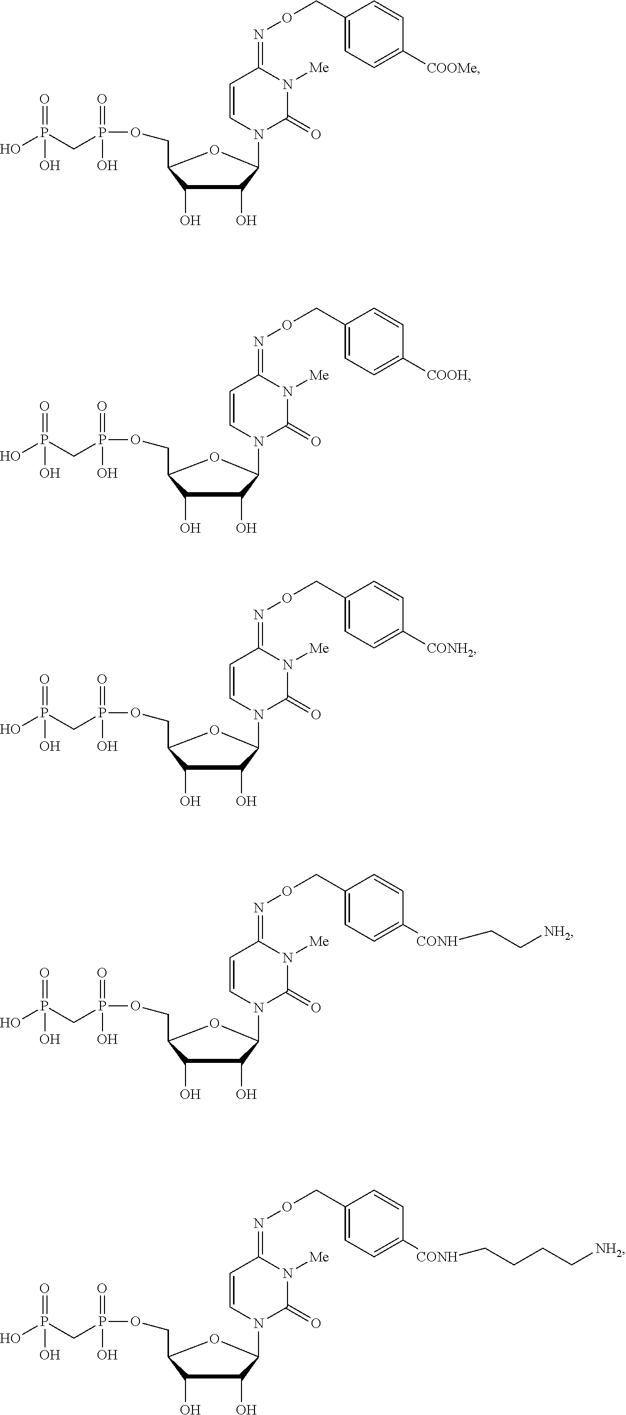

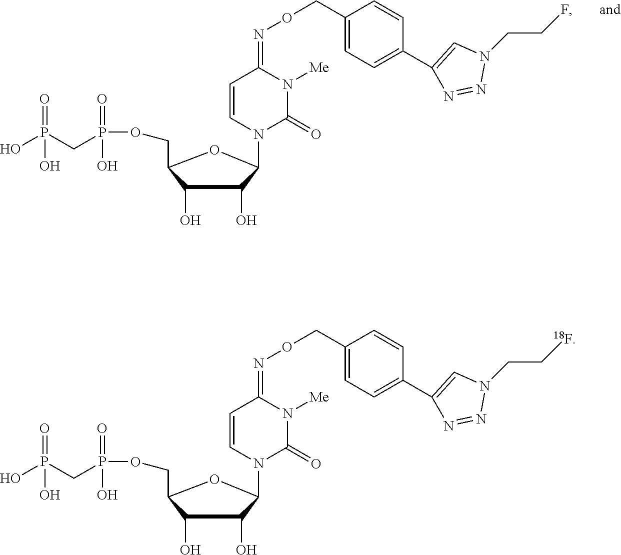

21. The compound or salt of embodiment 1 or 2, wherein T is

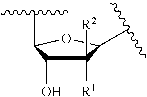

### and A is

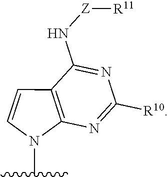 [0205] 22. The compound or salt of embodiment 21, wherein R¹ is OH and R² is H.
23. The compound or salt of embodiment 21 or 22, wherein R¹⁰ is H.
24. The compound or salt of any one of embodiments 21-23, wherein R⁵ is C₆-C₁₀ aryl.
25. The compound or salt of any one of embodiments 21-24, wherein Z is CH₂ or C₁H₄.
26. The compound or salt of any one of embodiments 21-25, wherein the compound is selected from the group consisting of:

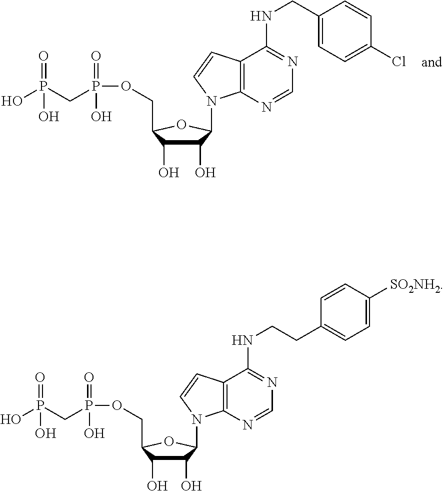

27. The compound or salt of embodiment 1, wherein Q is O, U is CH₂, T is

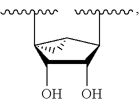

and A is

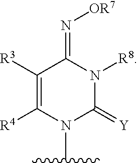

28. The compound or salt of embodiment 27, wherein the compound is:

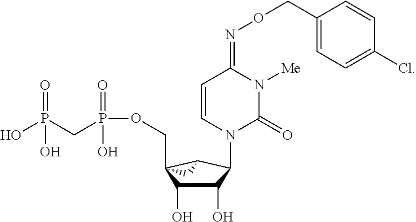

29. A pharmaceutical composition comprising the compound or salt of any one of embodiments 1-26 and a pharmaceutically acceptable carrier.
30. A method of inhibiting ecto-5′-nucleotidase in a mammal in need thereof, comprising administering to the mammal an effective amount of a compound or salt of any one of embodiments 1-28.
31. The method of embodiment 30, wherein the ecto-5′-nucleotidase is associated with a cancer cell.
32. A method of inhibiting suppression of an antitumor immune response in a mammal in need thereof, comprising administering to the mammal an effective amount of a compound or salt of any one of embodiments 1-28.
33. The method of embodiment 32, wherein the antitumor response is mediated via activation of adenosine 2A (A2A) receptors.
34. The method of embodiment 32 or 33, wherein the mammal is afflicted with lung cancer, prostate cancer, triple-negative breast cancer, melanoma, leukemia, or thyroid cancer.
35. A method of inhibiting tumor growth of a cancerous tumor in a mammal in need thereof, comprising administering to the mammal an effective amount of a compound or salt of any one of cla embodiments ims 1-28.
36. The method of embodiment 35, wherein the tumor is associated with lung cancer, prostate cancer, triple-negative breast cancer, or thyroid cancer.
37. A method of inhibiting metastasis of cancer in a mammal afflicted with cancer, comprising administering to the mammal an effective amount of a compound or salt of any one of embodiments 1-28.
38. The method of embodiment 37, wherein the tumor is associated with lung cancer, prostate cancer, triple-negative breast cancer, or thyroid cancer.
39. A method of synergistically enhancing a response of a mammal afflicted with cancer undergoing treatment with an immunotherapeutic anti-cancer agent, comprising co-administering to the mammal an effective amount of a compound or salt of any one of embodiments 1-28.
40. The method of embodiment 38, wherein the cancer is lung cancer, prostate cancer, triple-negative breast cancer, or thyroid cancer.
41. The method of embodiment 39 or 40, wherein the immunotherapeutic anti-cancer agent is selected from the group consisting of pembrolizumab, nivolumab, atezolizumab, durvalumab, and ipilimumab.
42. A method of potentiating an activity of an inhibitor of nicotinamide phosphoribosyltransferase in a mammal undergoing treatment of a mammal with the inhibitor, comprising administering to the mammal an effective amount of a compound or salt of any one of embodiments 1-28.
43. The method of embodiment 42, wherein the inhibitor of nicotinamide phosphoribosyltransferase is FK866.
44. A method of treating preeclampsia in in a mammal in need thereof, comprising administering to the mammal an effective amount of a compound or salt of any one of embodiments 1-28.
45. A method of treating cancer in a mammal comprising administering to the animal an effective amount of a compound of any one of embodiments 1-28, wherein the cancer is lung cancer, prostate cancer, triple-negative breast cancer, melanoma, leukemia, or thyroid cancer.
46. A method of imaging a mammal by use of positron emission tomography (PET), comprising administering to the mammal a compound of embodiment 1, wherein the compound is:

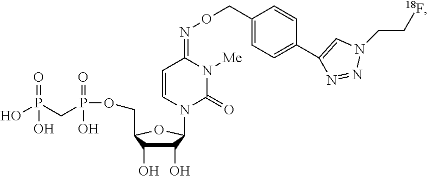

and then imaging the mammal.
47. A compound of the formula:

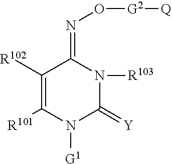

wherein R¹⁰¹ and R¹⁰² are independently H, halo, C₁-C₆ alkyl, C₁-C₆ aryl, NH₂, N₃, C₁-C₆ alkynyl, C₆-C₁₀ aryl-C₁-C₆ alkylenyl, C₆-C₁₀ heteroaryl-C₁-C₆ alkyl, C₆-C₁₀ heteroaryl or C₆-C₁₀ arylcarbonyl,
R¹⁰³ is H, C₁-C₆ alkyl, C₆-C₁₀ aryl-C₁-C₆ alkyl, C₆-C₁₀ aryl-C₁-C₆ alkylenyl, C₁-C₆ alkylcarbonyl, C₆-C₁₀ heteroaryl-C₁-C₆ alkyl, C₆-C₁₀ heteroaryl or C₆-C₁₀ arylcarbonyl,
G¹ is

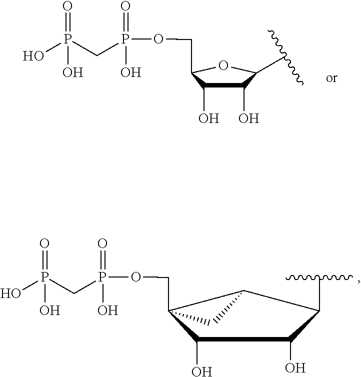

G² is

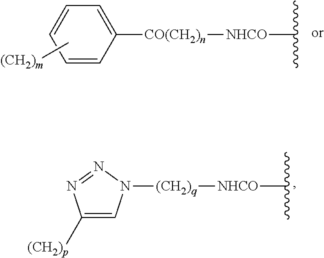

m, n, p, and q are independently integers of from 1 to about 20, and
Q is a fluorophore moiety;
or a pharmaceutically acceptable salt thereof
48. The compound or salt of embodiment 47, wherein Q is a fluorophore moiety selected from the group consisting of FITC, NBD, Dansyl, Squaraine Rotaxane, Bodipy FL, Bodipy TR, Bodipy 630/650-X, Bodipy 650/655-X, Texas Red, Cy5, 1-pyrene, EVOBlue 30, Alexa Fluor 532, Alexa Fluor 488-5, 488-6, or mixture thereof, Tamra, Tamra 5/6-X-SE, Alexa Fluor 488 azide 5 isomer, Alexa Fluor 488 5 isomer, Alexa Fluor 488 5/6 mixed isomers, NIR dye 700, NIR dye 800, Janelia Fluor 549 amide, and Janelia Fluor 646 amide.
49. The compound or salt of embodiment 47 or 48, wherein G¹ is

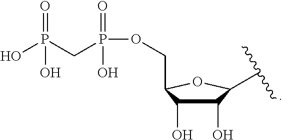

50. The compound or salt of embodiment 49, wherein G² is

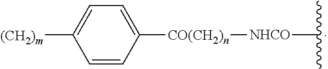

51. The compound or salt of embodiment 50, wherein the compound is:

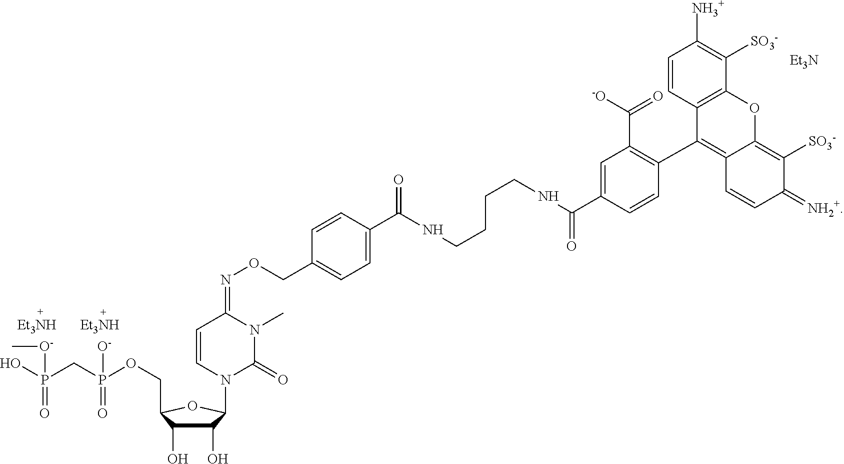

52. The compound or salt of embodiment 49, wherein G² is

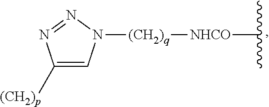

53. The compound or salt of embodiment 52, wherein the compound is:

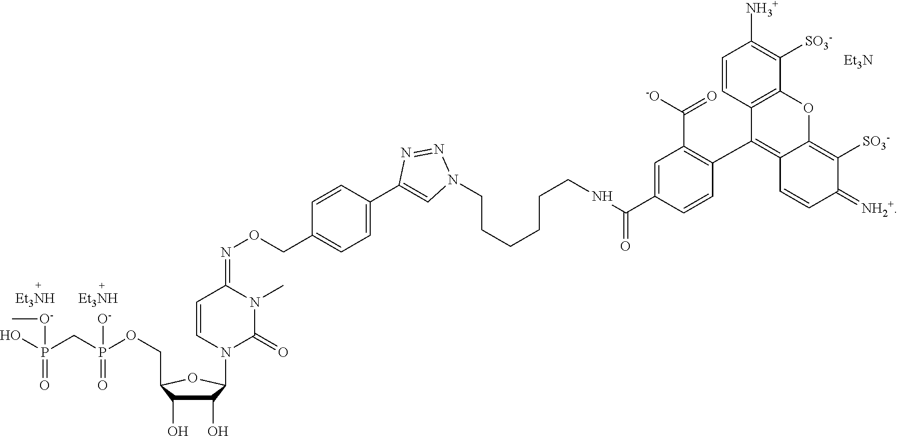

54. A diagnostic composition comprising a compound or salt of any one of embodiments 47-53 and a pharmaceutically acceptable carrier.

The following examples further illustrate the invention but, of course, should not be construed as in any way limiting its scope.

### General

### Reagents and Instrumentation.

All reagents were commercially obtained from various producers (Alfa Aesar, Carbosynth, and Sigma Aldrich) and used without further purification. The purity of all compounds including starting material was more than 95%, as determined using HPLC. Commercial solvents of specific reagent grades were used, without additional purification or drying. Analytical thin-layer chromatography was carried out on Sigma-Aldrich™ TLC plates and compounds were visualized with UV light at 254 nm. Silica gel flash chromatography was performed using 230-400 mesh silica gel. Unless noted otherwise, reagents and solvents were purchased from Sigma-Aldrich (St. Louis, Mo.). The ¹H, ³¹P, and ¹³C NMR spectra were recorded using Bruker 400 MHz spectrometer, a DD2 400 MHz or DD2 600 MHz NMR spectrometer (Agilent). DMSO-d₆, MeOD-d₄, CDCl₃ or D₂O were used as solvents. Shifts are given in ppm relative to the remaining protons of the deuterated solvents used as internal standard (¹H-, ¹³C-NMR). Purification of final compounds was performed by semi-preparative HPLC (Column: Luna 5 μm C₁₈(2) 100 Å, LC Column 250×4.6 mm). Eluent: 10 mM triethylammonium acetate buffer —CH₃CN from 80:20 to 20:80 in 40 min, with a flow rate of 5 mL/min. Purities of all tested compounds were ≥95%, as estimated by analytical HPLC: Method A: Eluent: 5 mM triethylammonium phosphate monobasic solution —CH₃CN from 100:0 to 50:50 in 20 min, then triethylammonium phosphate monobasic solution —CH₃CN to 100:0 in 5 min with a flow rate of 1 mL/min (Column: Zorbax SB-Aq 5 am analytical column, 50×4.6 mm; Agilent Technologies, Inc). Method B: Eluent: 5 mM triethylammonium phosphate monobasic solution —CH₃CN from 90:10 to 0:100 in 20 min, then triethylammonium phosphate monobasic solution —CH₃CN from 0:100 to 90:10 in 5 min with a flow rate of 1 mL/min (Column: Zorbax SB-Aq 5 am analytical column, 150×4.6 mm; Agilent Technologies, Inc). Method C: Eluent: 5 mM triethylammonium phosphate monobasic solution —CH₃CN from 80:20 to 20:80 in 20 min, then triethylammonium phosphate monobasic solution —CH₃CN from 20:80 to 80:20 in 10 min with a flow rate of 1 mL/min (Column: Zorbax SB-Aq 5 am analytical column, 150×4.6 mm; Agilent Technologies, Inc). Peaks were detected by UV absorption (254 nm) using a diode array detector. All derivatives tested for biological activity showed >95% purity in the HPLC system. Low-resolution mass spectrometry was performed with a JEOL SX102 spectrometer with 6-kV Xe atoms following desorption from a glycerol matrix or on an Agilent LC/MS 1100 MSD, with a Waters (Milford, Mass.) Atlantis C₁₈ column. High-resolution mass spectroscopic (HRMS) measurements were performed on a proteomics optimized Q-TOF-2 (Micromass-Waters) using external calibration with polyalanine. For lyophilization, a freeze dryer (Labconco FreeZone 4.5) was used.

### Preparation of Triethylammonium Hydrogen Carbonate Buffer

A 1 M solution of TEAC was prepared by adding dry ice slowly to 1 M triethylamine solution in deionized water for several hours until the pH of approximately 8.4-8.6 was indicated in pH-meter.

### Purification of Nucleotides

### Ion Exchange Chromatography

The crude nucleoside-5′-O-[(phosphonomethyl)phosphonic acid] derivatives were purified by ion exchange chromatography on an HPLC instrument UltiMate 3000 (Dionex Corp.) with a HiScale™ 26 20 BH, 26 mm×130 mm length column. The column was packed with Source 1SQ™ gel, swelled in a 20% EtOH-solution. Before running purification, the column was washed and equilibrated with deionized water. The sample was prepared by dissolving crude product in 0.5-1 mL of aqueous triethylammonium hydrogen carbonate buffer. Separation was achieved by running a solvent gradient of triethylammonium hydrogen carbonate buffer: deionized water from 0:100 for 5 min, then from 0:100 to 100:0 in 25 min, followed by a gradient from 110:0 to 0:100 in 20 min, and holding 0:100 for 10 min with a flow rate of 5 mL/min. The UV absorption was detected at 254 nm, 210 nm and 280 nm. Fractions were collected, and appropriate fractions pooled, diluted in water, and lyophilized.

### General Procedure a for the Synthesis of Nucleotides

To a solution of DCC (3 eq.) and nucleoside in DMF (2 mL) methylene diphosphonic acid (1.5 eq.) was added at rt and the mixture was allowed to stir at rt for 6-24 h. Samples were withdrawn at 3-12 h interval for LC-MS to check the disappearance of nucleosides and to monitor the formation of the desired nucleotide. On the disappearance of a nucleoside, 10 mL of cold TEAC-solution was added. The mixture was stirred at rt for 30 min followed by filtration and lyophilization of the aqueous solution. The mixture of nucleotide and dinucleotide was separated by ion-exchange chromatography on Source 15Q. Fractions containing the product were pooled and evaporated to dryness. The compound was then purified by RP-HPLC using a gradient of 10 mM triethylammonium acetate buffer —CH₃CN from 80:20 to 20:80 in 40 min, suitable fractions were pooled and lyophilized to obtain the final product as glassy solid.

### General Procedure B for the Synthesis of Nucleotides

A solution of methylenebis(phosphonic dichloride) (3 eq.) in trimethyl phosphate (2 mL), cooled to 0° C. was added to a suspension of the corresponding nucleoside in trimethyl phosphate at 0° C. The reaction mixture was stirred at 0° C. and samples were withdrawn at 10 min interval for LC-MS to check the disappearance of nucleosides. After 30 min, on the disappearance of a nucleoside, 7 mL of cold 1 M aqueous triethylammonium hydrogen carbonate buffer solution (pH 8.4-8.6) was added. It was stirred at 0° C. for 15 min followed by stirring at rt for 30 min. Trimethyl phosphate was extracted using (2×100 mL) of tert-butyl methyl ether, and the aqueous layer was lyophilized. The mixture of nucleotide and dinucleotide was separated by ion-exchange chromatography on Source 15Q. Fractions containing the product were pooled and evaporated to dryness. The compound was then purified by RP-HPLC using a gradient of 10 mM triethylammonium acetate buffer —CH₃CN from 80:20 to 20:80 in 40 min, then 10 mM triethylammonium acetate buffer —CH₃CN from 100:0 to 90:10 in 40 min, then 100:0 in 5 min, with a flow rate of 5 mL/min, suitable fractions were pooled and lyophilized to obtain final product as glassy solid.

Pharmacological Assays

Rat Ecto-5′-Nucleotidase Assay

The assay was performed as previously described (41) using recombinant rat CD73 expressed in Sf9 insect cells (33). The assay was performed by adding 10 μL of compound solution to 70 μL, of assay buffer (25 mM Tris, 140 mM sodium chloride and 50 mM sodium phosphate, pH 7.4), 10 μL of [³H]AMP (Hartmann Analytic, Germany) with a final substrate concentration of 5 μM and 10 μL of the enzyme solution (final concentration 0.3 ng/μL). The mixture was incubated for 25 min at 37° C. The reaction was stopped by adding 500 μL of ice-cooled precipitation buffer containing 100 mM aqueous lanthanum chloride and 100 mM aqueous sodium acetate solution (pH 4.0). After 30 min, the precipitation was filtered through GF/B glass fiber filters (M-48, Brandel, Gaithersburg, Md., USA). After washing three times, 4 mL of the scintillation cocktail ULTIMA Gold XR was added and then quantified by scintillation counting (TRICARB 2900 TR, Packard/PerkinElmer). Three separate inhibition assays were performed in triplicate, concentration-response curves were fitted, and Kᵢ values were calculated.

Soluble CD73 Enzyme Preparations

Soluble rat CD73 was expressed in Spodoptera frugiperda 9 (Sf9) insect cells and purified as previously described (33). The cDNA for the soluble human CD73 (Genbank accession no. NM_002526) was obtained from Prof. Dr. Norbert Sträter (University of Leipzig, Germany) (3). In order to generate a soluble enzyme the signaling sequence for anchoring the protein to the membrane via a GPI-anchor had been omitted (N-terminal residues: 1-27, C-terminal residues: 550-574 including GPI-anchor attachment site).^{K} In addition, a 6xHis-Tag was fused to the C-terminus and the construct was cloned into the vector pACGP67B, which provides an N-terminal signal peptide for the secretion of the protein. Sf9 insect cells were grown in Insect-XPRESS™ media (#: BE12-730Q, Lonza, Switzerland) with 10 mg/l gentamicin and split at a ratio of 1:3 every fourth day. For transfection, cells were seeded into cell culture flasks (25 cm²) at 60-70% confluence. 100 μl of cell medium and 1 μl of the vector DNA (1000 ng/μ1) were mixed with 2.5 μl of baculovirus genomic ProEasy™ vector DNA (AB vector, CA, USA) and combined with premixed 100 μl of cell medium and 8 μl of Cellfectin™ II Reagent (Thermo Fisher Scientific, MA, USA). The transfection mixture was left for 30 min at rt and then dropwise added to the cells into the cell culture flasks. The cells were incubated for 30 min at rt, and for further 4 days at 27° C. Cells from the transfection procedure were detached from the bottom of the flasks and centrifuged for 5 min at 2000 g. 1.5 ml of the supernatant (viral stock) was added to 75 cm² cell culture flasks containing Sf9 cells (60-70% confluence), and the cells were incubated for four days at 27° C. Then 1.5 ml of the supernatant were taken and added to uninfected Sf9 cells in a 75 cm² flask. This was repeated five more times, using more cells and larger flasks after the third round of infections (150 cm² to which 3.0 ml of supernatant were added).

The stock P8 was finally used for infection of the cells. For protein expression, 3 ml of the virus solution were used to infect 150 ml of cell media containing 2×10⁶ cells/ml in a 500 ml Erlenmeyer flask, and they were incubated for 4 days at 27° C. with shaking (150 rpm). Then, cell suspensions were transferred to 50 ml falcon tubes and centrifuged at 15 min at 5000 g at 4° C. The supernatants were subjected to ultrafiltration using Amicon® Ultra-15, 10 kDa cut-off (Merck Millipore, MA, USA) at 5000 g for 15-30 min at 4° C. The concentrated protein was purified with HisPur™ Ni²⁺-NTA spin columns (#: 88226, Thermo Fisher Scientific, MA, USA). The elution of the columns was performed as recommended in the instruction manual with adjusting the incubation time for protein binding to 1 h at 4° C. with an end-over-end mixer and an additional incubation step of 5 min with the elution buffer before eluting. Eluates were pooled and dialyzed (Membra-Cer, 14 kDa cut-off, 250 mm×44 mm×0.02 mm; Carl Roth, Germany) at 4° C. in 25 mM Tris, pH 7.4, with a volume adjusted to 40 times the volume of the elution fraction. The buffer was exchanged after 8 h. The enzyme was aliquoted and stored at −80° C. until use.

Membrane Preparation of CD73-Expressing Breast Cancer Cells

Triple-negative breast cancer cells (MDA-MB-231), which natively express CD73, were grown in Dulbecco's Modified Eagle Medium (DMEM, #: 41966, Thermo Fisher Scientific, MA, USA), to which 100 U/ml penicillin/100 μg/ml streptomycin (#: P06-07100, PAN Biotech, Germany) and 10% fetal bovine serum (FBS, #:P30-1502, PAN Biotech, Germany) had been added at 37° C. with 5% CO₂. Cells were split 1:20 every 72 h (at 80-90% cell confluence). To detach the adherent cells, growth media was removed, cells were washed with phosphate-buffered saline (PBS, for flasks: 2.5 ml, 5 ml, for 175 cm² flasks: 10 ml) and incubated with trypsin/EDTA (25 cm² flask: 1 ml, for larger flasks corresponding larger amounts) for 5 min in the incubator at 37° C. Detached cells were diluted with growth media (2 ml for 25 cm² flask) and transferred to new culture flasks containing growth media (25 cm² flasks: 5 ml). For membrane preparations, cells were expanded in 175 cm² culture flasks to 80-90% cell confluence. After detachment by trypsin/EDTA, 10⁶ cells per dish were transferred to cell culture dishes (150 cm²) and incubated for 4 days at 37° C. with 5% CO₂. The culture medium was removed, cells were washed with 10 ml of PBS and cells were frozen to −20° C. Afterwards, cells were treated with 1 ml of ice-cold buffer (50 mM Tris, 2 mM EDTA, pH 7.4), scraped off, collected in a conical tube and centrifuged for 10 min at 1000 g (4° C.). The supernatant was discarded, the pellet resuspended in membrane buffer (0.5 ml/dish; 25 mM Tris, 1 mM EDTA, 320 mM sucrose, 1:1000 protease inhibitor cocktail (#: P8340, Sigma-Aldrich, MO, USA), pH 7.4) and homogenized three times for 30 s each (20,500 rpm, Ultraturrax, IKA-Labortechnik, Germany). After a centrifugation step (10 min, 1000 g, 4° C.), the supernatants were collected and centrifuged for 30 min at 48,000 g (4° C.). The resulting supernatants were discarded, and the pellet was resuspended in washing puffer (0.5 ml/dish; 50 mM Tris, pH 7.4) and centrifuged again (same conditions). This step was repeated three times. Finally, the pellet was resuspended in washing buffer (0.1 ml/dish), aliquoted and stored at −80° C.

Enzyme Inhibition Assays

The assay was performed essentially as previously described.^{D} Stock solutions (10 mM) of the compounds were prepared in demineralized water and further dilutions were performed in assay reaction buffer (25 mM Tris, 140 mM sodium chloride, 25 mM sodium dihydrogen phosphate, pH 7.4). 10 μL of the inhibitor solution were added to 70 μL of assay reaction buffer. After the addition of 10 μL of CD73-containing solution or suspension (rat CD73: 1.63 ng; human CD73: 0.365 ng; membrane preparation of MDA-MB-231 cells expressing CD73: 7.4 ng of protein per vial), the reaction was initiated by the addition of 10 μL of [2,8-³H]AMP (specific activity 7.4×10⁸ Bq/mmol (20 mCi/mmol)), American Radio-labeled Chemicals, MO, USA, distributed by Hartman Analytic, Germany) resulting in a final substrate concentration of 5 μM. The enzymatic reaction was performed for 25 min at 37° C. in a shaking water bath. Then, 500 μl of cold precipitation buffer (100 mM lanthanum chloride, 100 mM sodium acetate, pH 4.0) were added to stop the reaction and to facilitate precipitation of free phosphate and unconverted [2,8-³H]AMP. After the precipitation was completed (after at least 30 min on ice), the mixture was separated by filtration through GF/B glass fiber filters using a cell harvester (M-48, Brandel, Gaitherburg, Md., USA). After washing each reaction vial three times with 400 μl of cold (4° C.) demineralized water, 5 ml of the scintillation cocktail (ULTIMA Gold XR, PerkinElmer, MA, USA) was added and radioactivity was measured by scintillation counting (TRICARB 2900 TR, Packard/PerkinElmer; counting efficacy: 49-52%). Two controls were included and measured as duplicates. One reaction was performed without the inhibitor resulting in 100% enzyme activity (positive control) and one was incubated without the inhibitor and the enzyme and served as background control. The resulting data were subtracted from the background and were normalized with to the positive control. The results were plotted, and concentration-inhibition curves were fitted with GraphPad Prism 5 (GraphPad Software, La Jolla, USA). The mean IC₅₀±SEM from three independent experiments was used to calculate the Kᵢ value with the Cheng-Prusoff equation^{L} (K_{m, rat CD73}: 53.0±4.1 μM; K_{m, human CD73}: 17.0±2.1 μM; K_{m, (MDA-MB-231)}: 14.8±2.1 μM)

Human P2Y₆ Receptor Assay

Calcium mobilization induced by the nucleotide derivatives was measured in a human astrocytoma cell line (1321N1) expressing the human P2Y₆ receptor, as previously described (33).

Human P2Y₁₄ Receptor Assay

Inhibition of binding of a high affinity fluorescent antagonist was measured using flow cytometry in a CHO cell line expressing the human P2Y₁₄ receptor, as previously described (34).

List of abbreviations: AR, adenosine receptor; ACN, acetonitrile; ADP, adenosine 5′diphosphate; AMP, adenosine 5′monophosphate; ATP, adenosine 5′triphosphate; AOPCP, α,β-methylene-ADP, adenosine-5′-O-[(phosphonomethyl)phosphonic acid], [{5-(6-aminopurin-9-yl)-3,4-dihydroxyoxolan-2-yl}methoxyhydroxyphosphoryl]methylphosphonic acid; CD73, cluster of differentiation 73; CE, capillary electrophoresis; DMEM, Dulbecco's Modified Eagle Medium; DMAP, 4-dimethylaminopyridine; DMF, N,N-dimethylformamide; DMSO, dimethyl sulfoxide; EC, enzyme commission; EDTA, ethylenediaminetetraacetic acid; ESI, electrospray ionization; FBS, fetal bovine serum; GPI, glycophosphatidylinositol; HPLC, high performance liquid chromatography; LC-MS, liquid chromatography-mass spectrometry; MeOD-d₄, deuterated methanol; MOE, Molecular Operating Environment; eN, ecto-5′-nucleotidase; eNPPs, ecto-nucleoside pyrophosphatases/phosphodiesterases; eNTPDases, ecto-nucleoside triphosphate diphosphohydrolases; PBS, phosphate-buffered saline; pdb, protein data bank; POMs, polyoxometalates; PSB, Pharmaceutical Sciences Bonn; SAR, structure-activity relationship; SF9, Spodoptera frugiperda 9; TEA, triethylamine; TEAC, triethylammonium hydrogencarbonate; THF, tetrahydrofuran, TLC, thin layer chromatography, TNBC, triple-negative breast cancer; Tris, tris(hydroxymethyl)aminomethane.

### Example 1

This example demonstrates the synthesis of compounds in accordance with an embodiment of the invention.

2′-Deoxyadenosine-5′-O-[(phosphonomethyl)phosphonic acid] (2a). Method A. The product was obtained as colorless solid after lyophilization (2 eq Et₃N-salt, 33.6 mg, 15%) ¹H NMR (400 MHz, D₂O): δ 8.41 (s, 1H), 8.16 (s, 1H), 6.41 (t, J=5.9 Hz, 1H), 4.17 (s, 1H), 4.08-3.90 (m, 3H), 3.10 (q, J=7.3 Hz, 11H), 2.76 (dt, J=13.3, 6.0 Hz, 1H), 2.55-2.43 (m, 1H), 2.04 (t, J=18.6 Hz, 2H), 1.18 (t, J=7.3 Hz, 16H). ³¹P NMR (160 MHz, D₂O): δ 21.8, 11.8. MS (ESI, m/z) 408.0 [M−H]⁻; ESI-HRMS calcd. m/z for C₁₁H₁₆N₅O₈P₂ 408.0474, found 408.0479 [m−h]−. HPLC purity 95% (Rₜ=4.8 min, Method HPLC-A).

2′-Amino-2′-deoxyadenosine-5′-O-[(phosphonomethyl)phosphonic acid] (2b). Method A. The product was obtained as colorless solid after lyophilization (0.5 eq Et₃N-salt, 1.5 mg, 0.6%). %). ¹H NMR (400 MHz, D₂O): δ 8.59 (s, 1H), 8.28 (s, 1H), 6.45 (d, J=7.4 Hz, 1H), 4.88-4.84 (m, 1H), 4.64 (t, J=5.6 Hz, 1H), 4.52 (s, 1H), 4.18 (q, J=11.7, 10.7 Hz, 2H), 3.20 (q, J=7.3 Hz, 3H), 2.19 (t, J=19.0 Hz, 2H), 1.28 (t, J=7.3 Hz, 5H). ¹³C NMR (100 MHz, D₂O): δ 155.7, 153.0, 149.3, 140.1, 118.8, 86.0 (d, J=6.0 Hz), 84.6, 70.1, 63.7, 56.3, 46.8 (2C), 27.6 (t, J=124.3 Hz), 8.3 (2C). ³¹P NMR (160 MHz, D₂O): δ 18.6, 14.6.MS (ESI, m/z) 423.1 [M−H]⁻; ESI-HRMS calcd. m/z for C₁₁H₁₇N₆O₈P₂ 423.0583, found 423.0590 [M−H]⁻. HPLC purity 98% (Rₜ=9.9 min, Method HPLC-B).

2′-Amino-2′-deoxyadenosine-3′-O-[(phosphonomethyl)phosphonic acid] (2c). The product was obtained as colorless solid after lyophilization (0.66 eq Et₃N-salt, 1.1 mg, 0.5%). %). ¹H NMR (400 MHz, D₂O): δ 8.37 (s, 1H), 8.28 (s, 1H), 6.50 (d, J=7.6 Hz, 1H), 5.22 (t, J=5.6 Hz, 1H), 4.79 (s, 1H), 4.59 (s, 1H), 3.94 (d, J=2.7 Hz, 2H), 3.21 (q, J=7.3 Hz, 4H), 2.29 (t, J=19.4 Hz, 2H), 1.28 (t, J=7.3 Hz, 6H). ¹³C NMR (100 MHz, D₂O): δ 155.9, 152.9, 148.7, 141.0, 119.5, 87.3, 86.4, 73.3, 61.3, 54.8, 46.8 (2C), 28.2 (t, J=124.4 Hz), 8.3 (2C). ³¹P NMR (160 MHz, D₂O): δ 18.8, 14.2. MS (ESI, m/z) 423.1 [M−H]⁻; ESI-HRMS calcd. m/z for C₁₁H₁₇N₆O₈P₂ 423.0583, found 423.0582 [M−H]⁻. HPLC purity 98% (Rₜ=6.9 min, Method HPLC-B).

3′-Deoxyadenosine-5′-O-[(phosphonomethyl)phosphonic acid] (2d). Method A. The product was obtained as colorless solid after lyophilization (1.5 eq Et₃N-salt, 2.5 mg, 6%) ¹H NMR (400 MHz, D₂O): δ 8.49 (s, 1H), 8.25 (s, 1H), 6.10 (s, 1H), 4.77-4.65 (m, 1H), 4.25 (d, J=10.2 Hz, 1H), 4.13-4.01 (m, 1H), 3.19 (q, J=7.3 Hz, 10H), 2.42 (ddd, J=14.5, 9.2, 5.7 Hz, 1H), 2.25-2.09 (m, 3H), 1.27 (t, J=7.3 Hz, 15H). ¹³C NMR (100 MHz, D₂O): δ 154.7, 151.4, 148.4, 140.3, 118.8, 90.6, 80.4 (d, J=7.3 Hz), 75.4, 64.9, 46.8 (4C), 33.0, 27.5 (t, J=124.5 Hz), 8.3 (4C). ³¹P NMR (160 MHz, D₂O): δ 18.4, 14.9. MS (ESI, m/z) 408.0 [M−H]⁻; ESI-HRMS calcd. m/z for C₁₁H₁₆N₅O₈P₂ 408.0474, found 408.0471 [M−H]⁻. HPLC purity 99% (Rₜ=17.1 min, Method HPLC-B).

1-Deazaadenosine-5′-O-[(phosphonomethyl)phosphonic acid] (2e). Method B. The product was obtained as colorless solid after lyophilization (1.5 eq Et₃N-salt, 1.7 mg, 8%). ¹H NMR (400 MHz, D₂O): δ 8.50 (s, 1H), 7.99 (d, J=5.9 Hz, 1H), 6.67 (d, J=5.9 Hz, 1H), 6.14 (d, J=5.9 Hz, 1H), 4.53 (s, 1H), 4.37 (s, 1H), 4.15 (s, 2H), 3.18 (q, J=7.3 Hz, 7H), 2.17 (t, J=19.5 Hz, 2H), 1.26 (t, J=7.3 Hz, 12H). ¹³C NMR (100 MHz, D₂O): δ 147.9, 145.3, 144.2, 139.7, 104.3, 87.0, 84.0 (d, J=6.2 Hz), 73.9, 70.4, 63.7, 46.7 (4C), 27.4, 8.3 (4C). ³¹P NMR (160 MHz, D₂O): δ 18.6, 14.6. MS (ESI, m/z) 425.0 [M+H]⁺; ESI-HRMS calcd. m/z for C₁₂H₁₉N₄O₉P₂ 425.0622, found 425.0626 [M+H]⁺. HPLC purity 98% (Rₜ=8 min, Method HPLC-C).

3-Deazaadenosine-5′-O-[(phosphonomethyl)phosphonic acid] (20. Method B. ¹H NMR (400 MHz, D₂O): 8.58 (s, 1H), 7.75 (s, 1H), 7.32 (d, J=6.2 Hz, 1H), 6.06 (d, J=6.2 Hz, 1H), 4.69 (t, J=5.9 Hz, 1H), 4.58-4.52 (m, 1H), 4.41 (s, 1H), 4.20 (s, 2H), 3.21 (q, J=7.3 Hz, 3H), 2.17 (t, J=19.3 Hz, 3H), 1.28 (t, J=7.3 Hz, 6H). ³¹P NMR (160 MHz, D₂O): δ 18.8, 14.4. MS (ESI, m/z) 423.1 [M−H]₋; ESI-HRMS calcd. m/z for C₁₂H₁₇N₄O₉P₂ 423.0476, found 423.0473 [M−H]⁻. HPLC purity 97% (Rₜ=9.9 min, Method HPLC-C).

7-Deazaadenosine-5′-O-[(phosphonomethyl)phosphonic acid] (2g). Method B. The product was obtained as colorless solid after lyophilization (1 eq Et₃N-salt, 13.5 mg, 14%). ¹H NMR (600 MHz, D₂O): δ 8.13 (bs, 1H), 7.61 (s, 1H), 6.70 (s, 1H), 6.14 (d, J=5.4 Hz, 1H), 4.52 (t, J=5.5 Hz, 1H), 4.47 (t, J=4.4 Hz, 1H), 4.36-4.31 (m, 1H), 4.29 (d, J=11.6 Hz, 1H), 4.17 (d, J=11.5 Hz, 1H), 3.18 (q, J=7.3 Hz, 6H), 2.24 (t, J=20.5 Hz, 2H), 1.26 (t, J=7.3 Hz, 9H). ¹³C NMR (100 MHz, D₂O): δ 150.8, 146.9, 142.9, 124.6, 102.6, 102.1, 86.7, 83.7 (d, J=5.4 Hz), 74.6, 70.3, 63.9, 46.7 (3C), 27.6 (t, J=124.5 Hz), 8.3 (3C). ³¹P NMR (160 MHz, D₂O): δ 18.5, 15.1. MS (ESI m/z) 423.1 [M−H]⁻; ESI-HRMS calcd. m/z for C₁₂H₁₇N₄O₉P₂ 423.0476, found 423.0476 [M−H]⁻. HPLC purity >99% (Rₜ=4.3 min, Method HPLC-C).

Uridine-5′-O-[(phosphonomethyl)phosphonic acid] (4a). Method A. The product was obtained as colorless solid after lyophilization (2 eq Et₃N-salt, 6 mg, 5%). ¹H NMR (400 MHz, D₂O): δ 7.91 (d, J=8.1 Hz, 1H), 5.90-5.80 (m, 2H), 4.34-4.24 (m, 2H), 4.17-4.14 (m, 1H), 4.13-4.02 (m, 2H), 3.10 (q, J=7.3 Hz, 12H), 2.04 (t, J=19.6 Hz, 2H), 1.17 (t, J=7.3 Hz, 18H). ¹³C NMR (100 MHz, D₂O): δ 166.5 152.0, 141.9, 102.6, 88.7, 83.4 (d, J=6.9 Hz), 73.9, 69.5, 63.3, 46.8 (6C), 27.8 (t, J=122.4 Hz), 8.3 (6C). ³¹P NMR (160 MHz, D₂O): δ 20.6, 12.7. MS (ESI, m/z) 401.0 [M−H]⁻; ESI-HRMS calcd. m/z for C₁₀H₁₅N₂O₁₁P₂ 401.0157, found 401.0154 [M−H]⁻. HPLC purity 99% (Rₜ=5.6 min, Method HPLC-B).

3-Methyluridine-5′-O-[(phosphonomethyl)phosphonic acid] (4b). Method A. The product was obtained as colorless solid after lyophilization (2 eq Et₃N-salt, 4.8 mg, 19%). ¹H NMR (400 MHz, D₂O): δ 8.00 (d, J=8.0 Hz, 1H), 6.02 (d, J=8.0 Hz, 1H), 5.98 (d, J=3.0 Hz, 1H), 4.38 (d, J=3.6 Hz, 2H), 4.27 (s, 1H), 4.20 (s, 2H), 3.30 (s, 3H), 3.21 (q, J=7.3 Hz, 12H), 2.12 (t, J=19.3 Hz, 2H), 1.29 (t, J=7.3 Hz, 18H). ¹³C NMR (100 MHz, D₂O): δ 165.7, 152.3, 139.7, 101.8, 89.8, 83.1, 74.0, 69.3, 63.1, 46.8 (6C), 27.8, 8.3 (6C). ³¹P NMR (160 MHz, D₂O): δ 20.2, 13.2. MS (ESI, m/z) 415.1 [M−H]⁻; ESI-HRMS calcd. m/z for C₁₁H₁₇N₂O₁₁P₂ 415.0308, found 415.0311 [M−H]⁻. HPLC purity 96% (Rₜ=3.7 min, Method HPLC-C).

3-Ethyluridine-5′-O-[(phosphonomethyl)phosphonic acid] (4c). Method A. The product was obtained as colorless solid after lyophilization (2 eq Et₃N-salt, 26.1 mg, 11%). ¹H NMR (400 MHz, D₂O): δ 8.01 (d, J=7.1 Hz, 1H), 6.00 (d, J=6.5 Hz, 1H), 5.97-5.94 (m, 1H), 4.47-4.34 (m, 2H), 4.30-4.16 (m, 3H), 3.93 (q, J=6.9 Hz, 2H), 3.19 (q, J=7.4 Hz), 2.19-1.94 (m, 2H), 1.27 (t, J=7.3 Hz), 1.18 (t, J=7.1 Hz). ¹³C NMR (100 MHz, D₂O): δ 165.3, 151.8, 139.8, 102.0, 89.8, 83.1, 74.1, 69.0, 62.9, 46.7 (6C), 36.8, 11.8, 8.3 (6C). The signal for PCH₂P could not be observed. ³¹P NMR (160 MHz, D₂O): δ 21.1, 12.0. MS (ESI, m/z) 429.1 [M−H]⁻; ESI-HRMS calcd. m/z for C₁₂H₁₉N₂O₁₁P₂ 429.0470, found 429.0470 [M−H]⁻. HPLC purity 97% (Rₜ=9.8 min, Method HPLC-C).

3-Propyluridine-5′-O-[(phosphonomethyl)phosphonic acid] (4d). Method A. The product was obtained as colorless solid after lyophilization (2 eq Et₃N-salt, 11 mg, 5%). ¹H NMR (400 MHz, D₂O): δ 8.00 (d, J=7.8 Hz, 1H), 6.03 (d, J=7.7 Hz, 1H), 6.00 (d, J=3.5 Hz, 1H), 4.43-4.39 (m, 2H), 4.32-4.27 (m, 1H), 4.26-4.13 (m, 2H), 3.87 (dd, J=8.7, 6.6 Hz, 2H), 3.22 (q, J=7.3 Hz, 12H), 2.30-2.11 (m, 2H), 1.64 (dq, J=14.8, 7.5 Hz, 2H), 1.30 (t, J=7.3 Hz, 18H), 0.92 (t, J=7.5 Hz, 3H). ¹³C NMR (100 MHz, D₂O): δ 165.5, 152.1, 139.8, 102.1, 89.5, 83.3, 73.9, 69.5, 63.3, 46.8 (6C), 43.2, 27.2 (1C, PCH₂P), 20.3, 10.6, 8.3 (6C). The signal for PCH₂P could not be observed in 1D experiment. ¹³C-NMR shift of PCH₂P was determined using HSQC. ³¹P NMR (160 MHz, D₂O): δ 18.4, 14.5. MS (ESI, m/z) 443.1 [M−H]⁻; ESI-HRMS calcd. m/z for C₁₃H₂₁N₂O₁₁P₂ 443.0626, found 443.0634 [M−H]⁻. HPLC purity 95% (Rₜ=10.3 min, Method HPLC-C).

3-Benzyluridine-5′-O-[(phosphonomethyl)phosphonic acid] (4e). Method A. The product was obtained as colorless solid after lyophilization (2 eq Et₃N-salt, 3.7 mg, 2%). ¹H NMR (400 MHz, D₂O): δ 8.07 (d, J=7.9 Hz, 1H), 7.44-7.38 (m, 2H), 7.38-7.32 (m, 3H), 6.09 (d, J=7.9 Hz, 1H), 5.99 (d, J=3.7 Hz, 1H), 5.16 (d, J=15.1 Hz), 5.11 (d, J=15.2 Hz), 4.39 (d, J=3.9 Hz, 2H), 4.30-4.26 (m, 1H), 4.20 (q, J=11.4 Hz, 2H), 3.20 (q, J=7.3 Hz, 12H), 2.20 (t, J=18.3 Hz, 2H), 1.28 (t, J=7.3 Hz, 18H). ¹³C NMR (100 MHz, D₂O): δ 165.2, 152.1, 140.1, 136.1, 128.9 (2C), 127.8, 127.1 (2C), 102.1, 89.5, 83.3 (1C), 74.0, 69.5, 63.2, 46.7 (6C), 44.6, 27.5 (t, J=124.1 Hz, 1C, PCH₂P), 8.3 (6C). ³¹P NMR (160 MHz, D₂O): δ 18.4, 14.9. MS (ESI, m/z) 491.1 [M−H]⁻; ESI-HRMS calcd. m/z for for C₁₇H₂₁N₂O₁₁P₂ 491.0626, found 491.0622 [M−H]⁻. HPLC purity 98% (Rₜ=11.2 min, Method HPLC-C).

5-Methyluridine-5′-O-[(phosphonomethyl)phosphonic acid] (4f). Method A. The product was obtained as colorless solid after lyophilization (2 eq Et₃N-salt, 54 mg, 24%). ¹H NMR (400 MHz, D₂O): δ 7.77 (s, 1H), 5.99 (d, J=5.0 Hz, 1H), 4.46-4.34 (m, 2H), 4.26 (s, 1H), 4.16 (q, J=4.7, 4.1 Hz, 2H), 3.20 (q, J=7.3 Hz, 12H), 2.20 (t, J=19.7 Hz, 2H), 1.94 (s, 3H), 1.28 (t, J=7.3 Hz, 18H). ¹³C NMR (100 MHz, D₂O): δ 166.6, 152.1, 137.3, 111.9, 88.1, 83.6 (d, J=8.0 Hz), 73.5, 69.9, 63.6 (d, J=4.3 Hz), 59.0, 46.7 (6C), 27.6 (t, J=124.2 Hz), 11.7, 8.3 (6C), 7.5. ³¹P NMR (160 MHz, D₂O): δ 18.1 (d, J=9.4 Hz), 14.7 (d, J=9.4 Hz). MS (ESI, m/z) 415.0 [M−H]⁻; ESI-HRMS calcd. m/z for C₁₁H₁₇N₂O₁₁P₂ 415.0308, found 415.0302 [M−H]⁻. HPLC purity 95% (Rₜ=2.9 min, Method HPLC-B).

Thymidine-5′-O-[(phosphonomethyl)phosphonic acid] (4g). Method B. The product was obtained as colorless solid after lyophilization (2 eq Et₃N-salt, 28.35 mg, 12%). ¹H NMR (400 MHz, D₂O): δ 7.74 (d, J=1.3 Hz, 1H), 6.34 (dd, J=7.5, 6.3 Hz, 1H), 4.62 (dt, J=6.4, 3.4 Hz, 1H), 4.19-4.14 (m, 1H), 4.14-4.07 (m, 2H), 3.20 (q, J=7.3 Hz, 12H), 2.45-2.30 (m, 2H), 2.18 (t, J=19.8 Hz, 2H), 1.93 (s, 3H), 1.28 (t, J=7.3 Hz, 18H). ¹³C NMR (100 MHz, D₂O): δ 151.8, 137.5, 111.8, 109.9, 85.5 (d, J=7.2 Hz), 85.0, 71.0, 63.9, 46.7 (6C), 38.4, 26.9 (t, J=124.2 Hz), 11.7, 8.3 (6C). ³¹P NMR (160 MHz, D₂O): δ 18.1, 14.7. MS (ESI, m/z) 399.0 [M−H]⁻; ESI-HRMS calcd. m/z for C₁₁H₁₇N₂O₁₀P₂ 399.0358, found 399.0357 [M−H]⁻. HPLC purity 97% (Rₜ=9.3 min, Method HPLC-B).

2′-O-Methyl-5-methyluridine-5′-O-[(phosphonomethyl)phosphonic acid] (4h). Method A. The product was obtained as colorless solid after lyophilization (2 eq Et₃N-salt, 21 mg, 9%). ¹H NMR (400 MHz, D₂O): δ 7.79 (d, J=1.3 Hz, 1H), 6.03 (d, J=5.1 Hz, 1H), 4.53 (t, J=5.0 Hz, 1H), 4.26-4.22 (m, 1H), 4.22-4.14 (m, 2H), 4.13 (t, J=5.2 Hz, 1H), 3.49 (s, 3H), 3.20 (q, J=7.3 Hz, 12H), 2.20 (td, J=19.5, 2.0 Hz, 2H), 1.94 (d, J=1.2 Hz, 3H), 1.28 (t, J=7.3 Hz, 18H). ¹³C NMR (100 MHz, D₂O): δ 166.6, 151.8, 137.2, 111.9, 86.7, 83.6 (d, J=8.1 Hz), 82.4, 68.3, 63.4 (d, J=5.2 Hz), 58.1, 46.7 (6C), 27.5 (t, J=124.0 Hz), 11.7, 8.3 (6C). ³¹P NMR (160 MHz, D₂O): d 18.0, 14.7. MS (ESI, m/z) 429.0 [M−H]⁻; ESI-HRMS calcd. m/z for C₁₂H₁₉N₂O₁₁P₂ 429.0464, found 429.0465 HPLC purity 95% (Rₜ=9.5 min Method HPLC-B).

5-Ethynylyluridine-5′-O-[(phosphonomethyl)phosphonic acid] (4i). Method B. The product was obtained as brown solid after lyophilization (2 eq Et₃N-salt, 6.7 mg, 3%). ¹H NMR (600 MHz, D₂O): δ 8.26 (s, 1H), 5.92 (d, 1=4.0 Hz, 1H), 4.37 (dt, J=13.8, 4.9 Hz, 2H), 4.28-4.24 (m, 1H), 4.20 (d, J=10.0 Hz, 1H), 4.14 (d, J=11.6 Hz, 1H), 3.63 (s, 1H), 3.18 (q, J=7.3 Hz, 12H), 2.25-2.12 (m, 2H), 1.26 (t, J=7.3 Hz, 18H). ¹³C NMR (150 MHz, D₂O): δ 164.7, 150.7, 145.4, 98.8, 89.2, 83.7, 83.4, 74.5, 74.1, 69.4, 62.9, 46.7 (6C), 27.3 (t, J=126.5 Hz), 8.3 (6C). ³¹P NMR (160 MHz, D₂O): δ 17.1 (br). MS (ESI, m/z) 429.0 [M−H]⁻; ESI-HRMS calcd. m/z for C₁₂H₁₅N₂O₁₁P₂ 425.0157, found 425.0165 [M−H]⁻ HPLC purity 99% (Rₜ=8.6 min Method HPLC-C).

5-(1-Chlorovinyl)uridine-5′-O-[(phosphonomethyl)phosphonic acid] (4j) The compound was obtained as a side product during the synthesis of compound 4i via Method B. The product was obtained as brown solid after lyophilisation (3 eq Et₃N-salt, 2.7 mg, 1%). ¹H NMR (600 MHz, D₂O): δ 8.10 (d, J=0.7 Hz, 1H), 6.01 (dd, J=1.6, 0.7 Hz, 1H), 5.95 (d, J=5.1 Hz, 1H), 5.71 (d, J=1.7, 0.8 Hz, 1H), 4.42 (t, J=5.2 Hz, 1H), 4.35 (t, J=4.9 Hz, 1H), 4.27 (q, J=3.8 Hz, 1H), 4.17-4.13 (m, 2H), 3.18 (q, J=7.3 Hz, 18H), 2.18 (t, J=19.9 Hz, 2H), 1.26 (t, J=7.3 Hz, 27H). ¹³C NMR (150 MHz, D₂O): δ 163.1, 151.0, 140.8, 130.3, 119.0, 113.1, 89.2, 83.7 (d, J=8.1), 73.8, 69.8, 63.4 (d, J=5.4), 46.7 (9C), 27.5 (t, J=124.5), 8.3 (9C). ³¹P NMR (160 MHz, D₂O): δ 18.1, 15.2. MS (ESI, m/z) 461.0 [M−H]⁻; ESI-HRMS calcd. m/z for C₁₂H₁₆C₁N₂O₁₁P₂ 460.9923, found 460.9923 [M−H]⁻. HPLC purity 95% (Rₜ=9.8 min, Method HPLC-C).

5-(1-Chlorovinyl)-3-methyluridine-5′-O-[(phosphonomethyl)phosphonic acid] (4k) The compound was obtained as a side product during the synthesis of compound 4i via Method B. The product was obtained as brown solid after lyophilisation (3 eq Et₃N-salt, 5.6 mg, 2%). ¹H NMR (600 MHz, D₂O): δ 8.10 (d, J=0.8 Hz, 1H), 5.97 (dd, J=4.6, 0.9 Hz, 1H), 5.96 (dd, J=1.6, 0.7 Hz, 1H), 5.71 (dd, J=1.6, 1.0 Hz, 1H), 4.41 (t, J=5.0 Hz, 1H), 4.34 (t, J=5.2 Hz, 1H), 4.27 (q, J=3.9 Hz, 1H), 4.20-4.12 (m, 2H), 3.30 (s, 3H), 3.18 (q, J=7.3 Hz, 18H), 2.18 (t, J=19.6 Hz, 2H), 1.26 (t, J=7.3 Hz, 27H). ¹³C NMR (150 MHz, D₂O): δ 162.6, 151.4, 138.7, 131.0, 119.1, 112.5, 90.2, 83.4 (d, J=8.0 Hz), 73.9, 69.6, 63.3 (d, J=5.1 Hz), 46.7 (9C), 28.1, 27.5 (t, J=125.0 Hz), 8.3 (9C). ³¹P NMR (160 MHz, D₂O): δ 18.0, 15.4. MS (ESI, m/z) 475.0 [M−H]⁻; ESI-HRMS calcd. m/z for C₁₃H₁₈C₁N₂O₁₁P₂ 475.0080, found 475.0093 [M−H]⁻. HPLC purity 95% (Rₜ=10.4 min, Method HPLC-C).

5-Fluorouridine-5′-O-[(phosphonomethyl)phosphonic acid] (41). Method B. The product was obtained as a colorless solid after lyophilisation (2 eq Et₃N-salt, 32.2 mg, 6%). ¹H NMR (600 MHz, D₂O): δ=8.19 (d, J=6.5 Hz, 1H), 5.96 (dt, J=2.7, 1.6 Hz, 1H), 4.41-4.37 (m, 2H), 4.31-4.26 (m, 1H), 4.23 (ddd, J=11.7, 4.8, 2.7 Hz, 1H), 4.17 (ddd, J=11.7, 5.6, 3.0 Hz, 1H), 3.22 (q, J=7.3 Hz, 12H), 2.24 (t, J=19.9 Hz, 2H, PCH₂P), 1.29 (t, J=7.3 Hz, 18H). ¹³C NMR (150 MHz, D₂O): δ 159.5 (d, J=26.0 Hz), 150.4, 140.9 (d, J=233.4 Hz), 125.7 (d, J=34.8 Hz), 88.8, 83.5 (d, J=8.0 Hz), 73.9, 69.5, 63.2 (d, J=5.3 Hz), 46.7 (6C), 27.4 (t, J=125.0 Hz, PCH₂P), 8.3 (6C). ³¹P NMR (160 MHz, D₂O): δ 18.0, 15.4. MS (ESI, m/z) 419.0 [M−H]⁻; ESI-HRMS calcd. m/z for C₁₀H₁₄FN₂O₁₁P₂ 419.0062, found 419.0057 [M−H]⁻. HPLC purity 97% (Rₜ=9.3 min, Method HPLC-C).

5-Chlorouridine-5′-O-[(phosphonomethyl)phosphonic acid] (4m). Method B. The product was obtained as colorless solid after lyophilization (2 eq Et₃N-salt, 23.1 mg, 10%). ¹H NMR (400 MHz, D₂O): δ 8.19 (d, J=0.7 Hz, 1H), 5.92 (d, J=4.4 Hz, 1H), 4.40-4.34 (m, 2H), 4.28-4.24 (m, 1H), 4.19 (ddd, J=11.6, 4.6, 2.7 Hz, 1H), 4.13 (ddd, J=11.8, 5.7, 3.1 Hz, 1H), 3.18 (q, J=7.3 Hz, 12H), 2.22 (t, J=19.9 Hz, 2H), 1.26 (t, J=7.4 Hz, 18H). ³¹P NMR (160 MHz, D₂O): δ 18.2, 14.9. MS (ESI, m/z) 435.0 [M−H]⁻; ESI-HRMS calcd. m/z for C₁₀H₁₄C₁N₂O₁₁P₂ 434.9767, found 434.9776 [M−H]⁻. HPLC purity 97% (Rₜ=8.9 min, Method HPLC-C).

5-Bromouridine-5′-O-[(phosphonomethyl)phosphonic acid] (4n). Method B. The product was obtained as colorless solid after lyophilization (2 eq Et₃N-salt, 4.1 mg, 2%). ¹H NMR (400 MHz, D₂O): δ 8.29 (s, 1H), 5.96 (d, J=4.5 Hz, 1H), 4.46-4.38 (m, 2H), 4.32-4.28 (m, 1H), 4.25-4.16 (m, 2H), 3.22 (q, J=7.3 Hz, 12H), 2.25 (t, J=18.7 Hz, 2H, PCH₂P), 1.30 (t, J=7.3 Hz, 18H). ¹³C NMR (150 MHz, D₂O): δ 162.0, 151.2, 141.0, 97.0, 89.0, 83.6, 74.0, 69.6, 63.1, 46.8 (6C), 27.5 (PCH₂P), 8.3 (6C). The signal for PCH₂P could not be observed in 1D experiment. ¹³C-NMR shift of PCH₂P was determined using HSQC. ³¹P NMR (160 MHz, D₂O): δ 18.6, 14.8. MS (ESI, m/z) 479.0 [M−H]⁻; ESI-HRMS calcd. m/z for C₁₀H₁₄⁷⁹BrN₂O₁₁P₂ 478.9262, found 478.9264 [M−H]⁻. HPLC purity 95% (Rₜ=9.3 min, Method HPLC-C).

5-Iodouridine-5′-O-[(phosphonomethyl)phosphonic acid] (4o). Method A. The product was obtained as colorless solid after lyophilization (2 eq Et₃N-salt, 17.5 mg, 9%). ¹H NMR (400 MHz, D₂O): δ 8.27 (s, 1H), 5.93 (d, J=4.7 Hz, 1H), 4.39 (dt, J=13.0, 5.1 Hz, 2H), 4.31-4.21 (m, 1H), 4.22-4.10 (m, 2H), 3.21 (q, J=7.3 Hz, 12H), 2.27 (t, J=19.7 Hz, 2H), 1.28 (t, J=7.3 Hz, 18H). ¹³C NMR (100 MHz, D₂O): δ 163.6, 151.9, 145.9, 88.9, 83.6 (d, J=8.4 Hz), 73.9, 69.7, 68.7, 63.2, 46.7 (6C), 28.0 (t, J=124.0 Hz), 8.3 (6C). ³¹P NMR (160 MHz, D₂O): δ 18.4, 14.7. MS (ESI, m/z) 526.9 [M−H]⁻; ESI-HRMS calcd. m/z for C₁₀H₁₄N₂O₁₁IP₂ 526.9118, found 526.9123 [M−H]⁻. HPLC purity 98% (Rₜ=9.6 min Method HPLC-B).

2′-Deoxyuridine-5′-O-[(phosphonomethyl)phosphonic acid] (4p). Method A. The product was obtained as colorless solid after lyophilization (2 eq Et₃N-salt, 16.2 mg, 13%). ¹H NMR (400 MHz, D₂O): δ 7.98 (d, J=8.1 Hz, 1H), 6.31 (t, J=6.8 Hz, 1H), 5.94 (d, J=8.1 Hz, 1H), 4.65-4.51 (m, 1H), 4.21-4.14 (m, 1H), 4.15-4.07 (m, 2H), 3.20 (q, J=7.3 Hz, 12H), 2.39 (dd, J=6.8, 5.0 Hz, 2H), 2.17 (t, J=19.9 Hz, 2H), 1.28 (t, J=7.3 Hz, 18H). ¹³C NMR (100 MHz, D₂O): δ 166.4, 151.7, 142.2, 102.5, 85.8 (d, J=7.5 Hz), 85.4, 70.9, 63.8 (d, J=4.7 Hz), 46.7 (6C), 38.8, 27.5 (t, J=124.0 Hz), 8.3 (6C). ³¹P NMR (160 MHz, D₂O): δ 18.3, 14.7. MS (ESI, m/z) 385.0 [M−H]⁻; ESI-HRMS calcd. m/z for C₁₀H₁₅N₂O₁₀P₂ 385.0202, found 385.0201 [M−H]⁻. HPLC purity 99% (Rₜ=16 min Method HPLC-B).

2′-Amino-2′-deoxyuridine-5′-O-[(phosphonomethyl)phosphonic acid] (4q). Method A. The product was obtained as colorless solid after lyophilization (1 eq Et₃N-salt, 4 mg, 8%), containing 8% of methylenediphosphonic acid. ¹H NMR (400 MHz, D₂O): δ 8.02 (d, J=8.1 Hz, 1H), 6.28 (d, J=7.6 Hz, 1H), 6.00 (d, J=8.1 Hz, 1H), 4.70 (d, J=4.6 Hz, 1H), 4.44 (s, 1H), 4.23-4.08 (m, 3H), 3.21 (q, J=7.3 Hz, 6H), 2.18 (td, J=19.8, 4.0 Hz, 2H), 1.28 (t, J=7.3 Hz, 9H). ³¹P NMR (160 MHz, D₂O): δ 18.5, 15.9 (8% methylenediphosphonic acid), 14.4. MS (ESI, m/z) 400.0 [M−H]⁻; ESI-HRMS calcd. m/z for C₁₀H₁₆N₃O₁₀P₂ 400.0311, found 400.0304 [M−H]⁻. HPLC purity 96% (Rₜ=1.8 min, Method HPLC-A).

2′-Azido-2′-deoxyuridine-5′-O-[(phosphonomethyl)phosphonic acid] (4r). Method A. The product was obtained as colorless solid after lyophilization (2 eq Et₃N-salt, 11 mg, 8%). ¹H NMR (400 MHz, D₂O): δ 8.02 (d, J=8.1 Hz, 1H), 6.02 (d, J=5.2 Hz, 1H), 5.97 (d, J=8.1 Hz, 1H), 4.62 (t, J=5.2 Hz, 1H), 4.40 (t, J=5.4 Hz, 1H), 4.27-4.09 (m, 3H), 3.20 (q, J=7.3 Hz, 12H), 2.19 (t, J=19.7 Hz, 2H), 1.28 (t, J=7.3 Hz, 18H). ¹³C NMR (100 MHz, D₂O): δ 166.3, 151.7, 141.6, 102.7, 87.1, 83.7 (d, J=8.0 Hz), 70.1, 65.4, 63.0 (d, J=3.8 Hz), 46.7 (6C), 27.5 (t, J=124.6 Hz), 8.3 (6C). ³¹P NMR (160 MHz, D₂O): δ 18.4, 14.6. MS (ESI, m/z) 426.0 [M−H]⁻; ESI-HRMS calcd. m/z for C₁₀H₁₄N₅O₁₀P₂ 426.0216, found 426.0222 [M−H]⁻. HPLC purity 99% (Rₜ=17.5 min, Method HPLC-B).

2′-Fluoro-2′-deoxyuridine-5′-O-[(phosphonomethyl)phosphonic acid] (4s). Method A. The product was obtained as colorless solid after lyophilization (2 eq Et₃N-salt, 25.3 mg, 10%). ¹H NMR (400 MHz, D₂O): δ 7.97 (d, J=8.1 Hz, 1H), 6.08 (dd, J=17.8, 1.8 Hz, 1H), 5.93 (d, J=8.1 Hz, 1H), 5.52 (ddd, J=52.5, 4.6, 1.8 Hz), 4.52 (ddd, J=21.6, 7.8, 4.6 Hz, 1H), 4.38-4.25 (m, 2H), 4.19 (ddd, J=11.7, 5.8, 2.8 Hz, 1H), 3.20 (q, J=7.3 Hz, 12H), 2.20 (t, J=19.7 Hz, 2H), 1.28 (t, J=7.3 Hz, 18H). ¹³C NMR (100 MHz, D₂O): δ 166.4, 151.4, 142.1, 102.3, 93.5 (d, J=185.9 Hz), 88.4 (d, J=35.1 Hz), 81.6 (d, J=7.9 Hz), 67.7 (d, J=15.9 Hz), 62.1 (d, J=4.9 Hz), 46.7 (6C), 27.5 (t, J=124.6 Hz), 8.3 (6C). ³¹P NMR (160 MHz, D₂O): δ 18.4, 14.7. MS (ESI, m/z) 403.0 [M−H]⁻; ESI-HRMS calcd. m/z for C₁₀H₁₄N₂O₁₀FP₂ 403.0108, found 403.0105 HPLC purity 98% (Rₜ=9.5 min, Method HPLC-B).

2′-ara-Fluoro-2′-deoxyuridine-5′-O-[(phosphonomethyl)phosphonic acid] (4t). Method A. The product was obtained as colorless solid after lyophilization (1.5 eq Et₃N-salt, 11 mg, 8%). ¹H NMR (400 MHz, D₂O): δ 7.93 (dd, J=8.1, 1.7 Hz, 1H), 6.32 (dd, J=15.5, 4.3 Hz, 1H), 5.92 (d, J=8.1 Hz, 1H), 5.23 (td, J=51.7, 3.6 Hz, 1H), 4.57 (dt, J=19.8, 3.6 Hz, 1H), 4.25-4.08 (m, 3H), 3.21 (q, J=7.3 Hz, 9H), 2.19 (t, J=19.7 Hz, 2H), 1.28 (t, J=7.3 Hz, 14H). ¹³C NMR (100 MHz, D₂O): δ 166.3, 151.4, 142.9, 101.9, 94.6 (d, J=191.8 Hz), 83.6 (d, J=16.9 Hz), 82.1, 73.2 (d, J=26.0 Hz), 62.7, 46.8 (4C), 27.5, 8.3 (4C). ³¹P NMR (160 MHz, D₂O): δ 20.6, 12.8. MS (ESI, m/z) 403.0 [M−H]⁻; ESI-HRMS calcd. m/z for C₁₀H₁₄N₂O₁₀FP₂ 403.0108, found 403.0112 [M−H]⁻. HPLC purity 98% (Rₜ=9.6 min, Method HPLC-B).

1(β-D-Arabinofuranosyl)-uridine-5′-O-[(phosphonomethyl)phosphonic acid] (4u). Method A. The product was obtained as colorless solid after lyophilization (2 eq Et₃N-salt, 21.3 mg, 9%). ¹H NMR (400 MHz, D₂O): 6=7.96 (d, J=8.1 Hz, 1H), 6.23 (d, J=5.5 Hz, 1H), 5.94 (d, J=8.1 Hz, 1H), 4.46 (t, J=5.5 Hz, 1H), 4.29 (t, J=6.0 Hz, 1H), 4.21 (tt, J=11.6, 6.6 Hz), 4.10 (dt, J=7.4, 3.9 Hz, 1H), 3.22 (q, J=7.3 Hz, 12H), 2.21 (t, J=19.7 Hz, 2H), 1.30 (t, J=7.3 Hz, 18H). ¹³C NMR (100 MHz, D₂O): δ 166.2, 151.4, 143.0, 101.3, 84.7, 81.2, 75.2, 73.8, 62.4, 46.7 (6C), 27.4 (t, J=124.8 Hz), 8.3 (6C). ³¹P NMR (160 MHz, D₂O): δ 18.4, 14.9. MS (ESI, m/z) 401.0 [M−H]⁻; ESI-HRMS calcd. m/z for C₁₀H₁₅N₂O₁₁P₂ 401.0157, found 401.0144 [M−H]⁻. HPLC purity 99% (Rₜ=8.6 min, Method HPLC-C).

6-Azauridine-5′-O-[(phosphonomethyl)phosphonic acid] (4v). Method A. The product was obtained as colorless solid after lyophilization (2 eq Et₃N-salt, 17.7 mg, 7%). ¹H NMR (400 MHz, D₂O): δ 7.66 (s, 1H), 6.15 (d, J=3.7 Hz, 1H), 4.65 (dd, J=5.1, 3.7 Hz, 1H), 4.48 (t, J=5.2 Hz, 1H), 4.30-4.21 (m, 1H), 4.16-3.98 (m 2H), 3.22 (q, J=7.3 Hz, 12H), 2.13 (t, J=17.8 Hz, 2H, PCH₂P), 1.30 (t, J=7.3 Hz, 18H). ¹³C NMR (100 MHz, D₂O): δ 158.6, 150.0, 137.2, 89.7, 83.1, 72.6, 70.5, 64.0, 46.7 (6C), 27.1 (1C, PCH₂P), 8.3 (6C). The signal of PCH₂P could not be observed in 1D experiment. ¹³C-NMR shift of PCH₂P was determined using HSQC. ³¹P NMR (160 MHz, D₂O): δ 18.3, 15.0. MS (ESI, m/z) 403.0 [M−H]⁻; ESI-HRMS calcd. m/z for C₉H₁₄N₃O₁₁P₂ 402.0109, found 402.0098 [M−H]⁻. HPLC purity 96% (Rₜ=9.1 min, Method HPLC-C).

Uridine-5′-O-[(phosphonoethyl)phosphonic acid] (4w). Method A. The product was obtained as colorless solid after lyophilization (3 eq Et₃N-salt and 1 eq H₃CCO₂H, 60 mg, 8%). ¹H NMR (400 MHz, D₂O): δ 7.93 (d, J=8.1 Hz, 1H), 6.14-5.71 (m, 2H), 4.32 (p, J=5.1 Hz, 2H), 4.28-4.22 (m, 1H), 4.17-4.01 (m, 2H), 3.18 (q, J=7.3 Hz, 18H), 1.90 (s, 3H, H₃CCO₂H), 1.85-1.64 (m, 4H), 1.26 (t, J=7.3 Hz, 27H). ¹³C NMR (100 MHz, D₂O): δ 181.0, 166.2, 151.8, 141.6, 102.6, 88.6, 83.4 (d, J=7.7 Hz), 73.9, 69.7, 63.1 (d, J=5.5 Hz), 46.7 (9C), 23.2, 22.1 (dd, J=133.5, 5.5 Hz), 20.4 (dd, J=135.4, 4.2 Hz), 8.3 (9C). ³¹P NMR (160 MHz, D₂O): δ 27.2 (d, J=73.5 Hz), 24.0 (d, J=73.5 Hz). MS (ESI, m/z) 415.0 [M−H]⁻; ESI-HRMS calcd. m/z for C₁₁H₁₇N₂O₁₁P₂ 415.0308, found 415.0311 [M−H]⁻. HPLC purity 99% (Rₜ=8.7 min, Method HPLC-B).

5-Methyluridine-5′-O-[(phosphonoethyl)phosphonic acid] (4x). Method A. The product was obtained as colorless solid after lyophilization (2 eq Et₃N-salt, 68 mg, 10%). ¹H NMR (400 MHz, D₂O): δ 7.74 (s, 1H), 5.99 (d, J=5.3 Hz, 1H), 4.44-4.32 (m, 2H), 4.30-4.23 (m, 1H), 4.16-4.03 (m, 2H), 3.20 (q, J=7.3 Hz, 12H), 1.94 (s, 3H), 1.90-1.65 (m, 4H), 1.28 (t, J=7.3 Hz, 17H). ¹³C NMR (100 MHz, D₂O): δ 166.6, 152.0, 137.1, 112.0, 88.1, 83.6 (d, J=7.9 Hz), 73.6, 70.0, 63.4 (d, J=5.5 Hz), 46.7 (6C), 22.0 (dd, J=133.4, 5.0 Hz), 20.4 (dd, J=134.8, 4.8 Hz), 11.8, 8.3 (6C). ³¹P NMR (160 MHz, D₂O): δ 27.3 (d, J=73.5 Hz), 24.1 (d, J=73.5 Hz). MS (ESI, m/z) 429.0 [M−H]⁻; ESI-HRMS calcd. m/z for C₁₂H₁₉N₂O₁₁P₂ 429.0464, found 429.0472 [M−H]⁻. HPLC purity 99% (Rₜ=9.1 min, Method HPLC-B).

2-Thiouridine-5′-O-[(phosphonomethyl)phosphonic acid] (4y). The product was obtained as colorless solid after lyophilization (1.5 eq Et₃N-salt). ¹H NMR (400 MHz, D₂O): δ 8.21 (d, J=8.2 Hz, 1H), 6.63 (d, J=2.8 Hz, 1H), 6.24 (d, J=8.1 Hz, 1H), 4.51-4.40 (m, 1H), 4.35 (t, J=5.7 Hz, 1H), 4.33-4.26 (m, 2H), 4.25-4.16 (m, 1H), 3.20 (q, J=7.4 Hz, 10H), 2.17 (t, J=19.6 Hz, 2H), 1.28 (t, J=7.3 Hz, 15H). ³¹P NMR (160 MHz, D₂O): δ 19.2, 14.0.

Cytidine-5′-O-[(phosphonomethyl)phosphonic acid] (7a). Method A. The product was obtained as colorless solid after lyophilization (2 eq Et₃N-salt, 10.3 mg, 8%). ¹H NMR (400 MHz, D₂O): δ 8.02 (d, J=7.6 Hz, 1H), 6.13 (d, J=7.5 Hz, 1H), 5.98 (d, J=4.1 Hz, 1H), 4.35 (dq, J=9.2, 5.0 Hz, 2H), 4.30-4.10 (m, 3H), 3.20 (q, J=7.3 Hz, 12H), 2.19 (t, J=19.7 Hz, 2H), 1.28 (t, J=7.3 Hz, 18H). ¹³C NMR (100 MHz, D₂O): δ 166.0, 157.4, 141.8, 96.5, 89.4, 82.9 (d, J=7.3 Hz), 74.3, 69.3, 63.1, 46.8 (6C), 27.5 (t, J=124.3 Hz), 8.3 (6C). ³¹P NMR (160 MHz, D₂O): δ 18.4, 14.6. MS (ESI, m/z) 403.0 [M−H]⁻; ESI-HRMS calcd. m/z for C₁₀H₁₆N₃O₁₀P₂ 400.0311, found 400.0309 [M−H]⁻. HPLC purity 96% (Rₜ=15.6 min, Method HPLC-B).

2′-Deoxycytidine-5′-O-[(phosphonomethyl)phosphonic acid] (7b). Method A. The product was obtained as colorless solid after lyophilization (2 eq Et₃N-salt, 34 mg, 14%). ¹H NMR (400 MHz, D₂O): δ 8.14 (d, J=7.7 Hz, 1H), 6.28 (t, J=6.5 Hz, 1H), 6.21 (d, J=7.7 Hz, 1H), 4.60 (dt, J=7.1, 3.8 Hz, 1H), 4.31-4.18 (m, 1H), 4.16-4.06 (m, 2H), 3.20 (q, J=7.3 Hz, 9H), 2.51-2.28 (m, 2H), 2.17 (t, J=19.8 Hz, 2H), 1.28 (t, J=7.3 Hz, 14H). ¹³C NMR (100 MHz, D₂O): δ 162.2, 152.4, 143.5, 95.7, 86.4, 86.1 (d, J=7.3 Hz), 70.7, 63.6 (d, J=4.3 Hz), 46.7 (4C), 39.5, 27.5 (t, J=124.7 Hz), 8.3 (4C). ³¹P NMR (160 MHz, D₂O): δ 18.3, 14.7. MS (ESI, m/z) 384.0 [M−H]⁻; ESI-HRMS calcd. m/z for C₁₀H₁₆N₃O₉P₂ 384.0362, found 384.0365 HPLC purity 99% (Rₜ=8.8 min, Method HPLC-B).

5-Iodocytidine-5′-O-[(phosphonomethyl)phosphonic acid] (7c). Method B. The product was obtained as colorless solid after lyophilization (1.75 eq Et₃N-salt, 9.4 mg, 5%). NMR (400 MHz, D₂O): δ 8.26 (s, 1H), 5.93 (d, 1H, J=3.2 Hz), 4.41-4.33 (m, 2H), 4.31-4.27 (m, 1H), 4.21 (m, 2H), 3.22 (q, J=7.3 Hz, 10.5H), 2.29 (t, J=17.9 Hz, 2H, PCH₂P), 1.30 (t, J=7.3 Hz, 15.75H). ¹³C NMR (100 MHz, D₂O): δ 164.6, 156.6, 147.6, 89.8, 83.1, 74.4, 69.2, 62.9, 46.7 (5.25C), 27.7 (PCH₂P), 8.3 (5.25C). The signal for PCH₂P could not be observed in 1D experiment. ¹³C-NMR shift of PCH₂P was determined using HSQC. ³¹P NMR (160 MHz, D₂O): δ 18.5, 15.1. MS (ESI, m/z) 525.9 [M−H]⁻; ESI-HRMS calcd. m/z for C₁₀H₁₆IN₃O₁₀P₂ 525.9283, found 525.9275 [M−H]⁻. HPLC purity 99% (Rₜ=9.0 min, Method HPLC-C.

5-Fluorocytidine-5′-O-[(phosphonomethyl)phosphonic acid] (7d). Method B. The product was obtained as colorless solid after lyophilisation (1.5 eq Et₃N-salt, 15.5 mg, 7%). ¹H NMR (600 MHz, D₂O): δ 8.15 (d, J=6.3 Hz, 1H), 5.90 (dd, J=3.9, 1.4 Hz, 1H), 4.34 (t, J=5.3 Hz, 1H), 4.30 (dd, J=5.1, 3.8 Hz, 1H), 4.27-4.19 (m, 2H), 4.14 (d, J=11.8 Hz, 1H), 3.18 (q, J=7.3 Hz, 9H), 2.19 (t, J=19.2 Hz, 2H), 1.26 (t, J=7.3 Hz, 15H). ¹³C NMR (150 MHz, D₂O): δ 158.1 (d, J=15.1 Hz), 155.4, 137.6 (d, J=248.3 Hz), 126.0 (d, J=33.0 Hz), 89.7, 82.9 (d, J=6.7 Hz), 74.4, 69.1, 62.9 (d, J=3.6 Hz), 46.7 (4.5C), 27.5 (t, J=119.5 Hz), 8.3 (4.5C). ³¹P NMR (160 MHz, D₂O): δ 18.9, 15.1. MS (ESI, m/z) 418.0 [M−H]⁻; ESI-HRMS calcd. m/z for C₁₀H₁₅FN₃O₁₀P₂ 418.0222, found 418.0235 [M−H]⁻. HPLC purity 98% (Rₜ=8.8 min, Method HPLC-C).

5-Methylcytidine-5′-O-[(phosphonomethyl)phosphonic acid] (7e). Method B. The product was obtained as colorless solid after lyophilisation (1.5 eq Et₃N-salt, 47.7 mg, 22%). ¹H NMR (600 MHz, D₂O): δ 7.92 (s, 1H), 5.99 (d, 1H, J=3.9 Hz), 4.42-4.35 (m, 2H), 4.32-4.25 (m, 1H), 4.25-4.13 (m, 2H, J=11.6 Hz), 3.21 (q, J=7.3 Hz, 9H), 2.21 (t, J=19.0 Hz, 2H, PCH₂P), 2.07 (s, 3H), 1.29 (t, J=7.3 Hz, 13.5H). ¹³C NMR (150 MHz, D₂O): δ 163.2, 153.8, 139.7, 89.2, 83.3, 74.2, 69.4, 63.3, 46.7 (4.5C), 27.5 (t, J=122.7 Hz, 1C, PCH₂P), 12.3, 8.3 (4.5C). ³¹P NMR (160 MHz, D₂O): δ 18.0, 14.8. MS (ESI, m/z) 414.0 [M−H]⁻; ESI-HRMS calcd. m/z for C₁₁H₁₉N₃O₁₀P₂ 414.0473, found 414.0484 [M−H]⁻. HPLC purity 99% (Rₜ=7.0 min, Method HPLC-C).

4-Benzoylcytidine-5′-O-[(phosphonomethyl)phosphonic acid] (71). Method B. The product was obtained as colorless solid after lyophilisation (5 eq Et₃N-salt, 39.2 mg, 13%). ¹H NMR (600 MHz, D₂O): δ 8.47 (d, J=7.0 Hz, 1H), 7.89 (d, J=7.7 Hz, 2H), 7.67 (t, J=7.4 Hz, 1H), 7.55 (t, J=7.7 Hz, 2H), 7.52-7.44 (m, 1H), 5.96-5.94 (m, 1H), 4.39-4.33 (m, 2H), 4.33-4.28 (m, 2H), 4.20 (d, J=11.6 Hz, 1H), 3.18 (q, J=7.3 Hz, 30H), 2.29-2.10 (m, 2H), 1.26 (t, J=7.3 Hz, 45H). ¹³C NMR (150 MHz, D₂O): δ 169.5, 163.2, 156.7, 145.8, 133.6, 132.6, 129.0 (2C), 128.1 (2C), 98.8, 90.9 (d, J=3.4 Hz), 82.8, 74.8, 68.7, 62.7, 46.7 (15C), 27.4 (t, J=120.2 Hz), 8.3 (15C). ³¹P NMR (160 MHz, D₂O): δ 18.5, 15.1. MS (ESI, m/z) 504.0 [M−H]⁻; ESI-HRMS calcd. m/z for C₁₇H₂₀N₃O₁₁P₂ 504.0579, found 504.0588 [M−H]⁻. HPLC purity 90% (Rₜ=10.5 min, Method HPLC-C). However, the compound displays decomposition in aqueous solution (see Supporting information) and was tested for its CD73 inhibition at a purity of 75%.

### N⁴-[O-(Benzyloxy)]-2′-deoxycytidine-5′-O-[(phosphonomethyl)phosphonic Acid] (9c)

Method B. The product was obtained as colorless solid after lyophilization (1.5 eq Et₃N-salt, 4.3 mg, 4.4%). ¹H NMR (400 MHz, D₂O): δ 7.50-7.37 (m, 5H), 7.20 (d, J=8.3 Hz, 1H), 6.28 (t, J=7.1 Hz, 1H), 5.71 (d, J=8.3 Hz, 1H), 5.03 (s, 2H), 4.57 (dt, J=5.9, 2.8 Hz, 1H), 4.16-3.97 (m, 3H), 3.19 (q, J=7.3 Hz, 9H), 2.33 (dt, J=14.1, 7.0 Hz, 1H), 2.23 (ddd, J=14.1, 6.4, 3.3 Hz, 1H), 2.14 (t, J=19.8 Hz, 2H), 1.27 (t, J=7.3 Hz, 14H). ¹³C NMR (100 MHz, D₂O): δ 147.2, 137.3, 132.3, 130.2, 128.8 (2C), 128.5, 128.4 (2C), 98.2, 85.1 (d, J=7.8 Hz), 84.3, 75.6, 71.2, 63.9, 46.7 (4C), 37.7, 13.6, 8.3 (4C). ³¹P NMR (160 MHz, D₂O): δ 21.3, 12.0. MS (ESI, m/z) 492.1 [M+H]⁻; ESI-HRMS calcd. m/z for C₁₇H₂₄N₃O₁₀P₂ 492.0937, found 492.0928 [M+H]⁻. HPLC purity 99% (Rₜ=10.9 min, Method HPLC-B).

N⁴-[O-(4-Trifluoromethylbenzyloxy)]-cytidine-5′-O-[(phosphonomethyl)phosphonic acid] (9d). Method B. The product was obtained as colorless solid after lyophilisation (2 eq Et₃N-salt, 32.1 mg, 17%). ¹H NMR (600 MHz, D₂O): δ 7.69 (d, J=8.0 Hz, 2H), 7.54 (d, J=8.0 Hz, 2H), 7.22 (d, J=8.1 Hz, 1H), 5.91 (d, J=4.8 Hz, 1H), 5.71 (d, J=8.1 Hz, 1H), 5.10 (s, 2H), 4.35-4.31 (m, 2H), 4.22-4.19 (m, 1H), 4.12-4.07 (m, 2H), 3.18 (q, J=7.3 Hz, 12H), 2.16 (t, J=18.8 Hz, 2H), 1.26 (t, J=7.3 Hz, 18H). ¹³C NMR (150 MHz, D₂O): δ 151.0, 147.0, 141.8, 132.2, 129.5 (q, J=31.9 Hz), 128.3 (2C), 125.5 (q, J=3.8 Hz, 2C), 124.3 (q, J=271.5 Hz), 98.3, 87.3, 83.4 (d, J=6.6 Hz), 74.5, 72.7, 70.2, 63.8, 46.7 (6C), 27.4 (t, J=124.4 Hz), 8.3 (6C). ³¹P NMR (160 MHz, D₂O): δ 18.2, 15.1. MS (ESI, m/z) 574.1 [M+H]⁻; ESI-HRMS calcd. m/z for C₁₈H₂₁F₃N₃O₁₁P₂ 574.0609, found 574.0616 [M−H]⁻. HPLC purity >99% (Rₜ=12.5 min, Method HPLC-C).

N⁴-[O-(Naphthalen-2-ylmethoxy)]-cytidine-5′-O-[(phosphonomethyl)phosphonic acid] (9e). Method B. The product was obtained as colorless solid after lyophilisation (2 eq Et₃N-salt, 4.5 mg, 2%). ¹H NMR (600 MHz, D₂O): δ 7.97-7.94 (m, 3H), 7.93-7.91 (m, 1H), 7.60-7.57 (m, 3H), 7.20 (d, J=8.2 Hz, 1H), 5.90 (d, J=5.5 Hz, 1H), 5.72 (d, J=8.2 Hz, 1H), 5.20 (s, 2H), 4.34-4.30 (m, 2H), 4.21-4.19 (m, 1H), 4.10-4.07 (m, 2H), 3.17 (q, J=7.3 Hz, 12H), 2.14 (t, J=19.2 Hz, 2H), 1.26 (t, J=7.3 Hz, 18H). ¹³C NMR (150 MHz, D₂O): δ 151.1, 147.0, 135.1, 133.0, 132.9, 132.1, 128.4, 128.0, 127.8, 127.2, 126.7, 126.6, 126.2, 98.4, 87.3, 83.4 (d, J=7.2 Hz), 75.5, 72.6, 70.2, 63.8, 46.7 (6C), 27.5 (t, J=127.0 Hz), 8.3 (6C). ³¹P NMR (160 MHz, D₂O): δ 18.3, 14.6. MS (ESI, m/z) 556.1 [M+H]⁻; ESI-HRMS calcd. m/z for C₂₁H₂₄N₃O₁₁P₂ 556.0892, found 556.0901 [M−H]⁻. HPLC purity 99% (Rₜ=12.2 min, Method HPLC-C).

N⁴-[O-(4-Benzyloxy)]-5-methyl-cytidine-5′-O-[(phosphonomethyl)phosphonic acid] (9t). Method B. The product was obtained as colorless solid after lyophilisation (2 eq Et₃N-salt, 17.5 mg, 9%). ¹H NMR (400 MHz, D₂O): δ 7.48-7.36 (m, 5H), 7.03-7.00 (m, 1H), 5.92-5.88 (m, 1H), 5.07 (s, 2H), 4.37-4.32 (m, 2H), 4.21-4.17 (m, 1H), 4.11-4.06 (m, 2H), 3.18 (q, J=7.3 Hz, 12H), 2.16 (t, J=19.6 Hz, 2H), 1.80 (s, 3H), 1.26 (t, J=7.3 Hz, 18H). ³¹p NMR (160 MHz, D₂O): δ18.0, 14.7. MS (ESI, m/z) 520.1 [M+H]⁻; ESI-HRMS calcd. m/z for C₁₈H₂₄N₃O₁₁P₂ 520.0892, found 520.0911 [M−H]⁻. HPLC purity >99% (Rₜ=10.9 min, Method HPLC-C).

N⁴-[O-(4-Benzyloxy)]-5-fluoro-cytidine-5′-O-[(phosphonomethyl)phosphonic acid] (9g). Method B. The product was obtained as colorless solid after lyophilisation (2 eq Et₃N- and 1 H₂CO₃-salt, 40.0 mg, 11%). ¹H NMR (600 MHz, D₂O): δ 7.45-7.36 (m, 6H), 5.89 (d, J=5.3 Hz, 1H), 5.09 (s, 2H), 4.35-4.31 (m, 1H), 4.29 (t, J=5.5 Hz, 1H), 4.24-4.17 (m, 1H), 4.14-4.07 (m, 2H), 3.57 (q, J=7.2 Hz, 6H), 3.17 (q, J=7.3 Hz, 6H), 2.24-2.05 (m, 2H), 1.33 (t, J=7.2 Hz, 9H), 1.26 (t, J=7.3 Hz, 9H). ¹³C NMR (150 MHz, D₂O): δ 149.7, 140.6 (d, J=21.7 Hz), 137.9 (d, J=235.3 Hz), 137.0, 128.8 (2C), 128.5, 128.3 (2C), 116.4 (d, J=35.1 Hz), 87.6, 83.4, 75.9, 72.8, 70.1, 63.7, 58.5 (3C), 46.7 (3C), 27.2 (t, J=124.4 Hz), 8.3 (3C), 7.19 (3C). ³¹P NMR (160 MHz, D₂O): δ 16.5. MS (ESI, m/z) 524.1 [M+H]⁻; ESI-HRMS calcd. m/z for C₁₇H₂₁FN₃O₁₁P₂ 524.0641, found 524.0645 [M−H]⁻. HPLC purity 99% (Rₜ=11.4 min, Method HPLC-C).

N⁴-[O-(4-Benzyloxy)]-3-methyl-cytidine-5′-O-[(phosphonomethyl)phosphonic acid] (9h). Method B. The product was obtained as colorless solid after lyophilisation (2 eq Et₃N-salt, 8.5 mg, 4%). ¹H NMR (600 MHz, D₂O): δ 7.48-7.44 (m, 2H), 7.44-7.41 (m, 2H), 7.40-7.37 (m, 1H), 7.32 (d, J=8.4 Hz, 1H), 6.44 (d, J=8.3 Hz, 1H), 5.94 (d, J=5.2 Hz, 1H), 5.01 (s, 2H), 4.34-4.30 (m, 2H), 4.20 (q, J=3.1 Hz, 1H), 4.12-4.08 (m, 2H), 3.21-3.14 (m, 15H), 2.16 (t, J=19.1 Hz, 2H), 1.26 (t, J=7.4 Hz, 18H). ¹³C NMR (150 MHz, D₂O): δ 153.9, 151.5, 136.9, 132.7, 128.9 (2C), 128.7 (2C), 128.5, 94.1, 88.4, 83.2 (d, J=7.3 Hz), 75.6, 73.0, 70.0, 63.7 (d, J=4.4 Hz), 46.7 (6C), 29.3, 27.4 (t, J=124.2 Hz), 8.3 (6C). ³¹P NMR (160 MHz, D₂O): δ 18.4, 15.0. MS (ESI, m/z) 520.1 [M+H]⁻; ESI-HRMS calcd. m/z for C₁₈H₂₄N₃O₁₁P₂ 520.0892, found 520.0889 [M−H]⁻. HPLC purity >99% (Rₜ=11.6 min, Method HPLC-C).

N⁴-[O-(4-Benzyloxy)]-3-ethyl-cytidine-5′-O-[(phosphonomethyl)phosphonic acid] (9i). Method B. The product was obtained as colorless solid after lyophilisation (2 eq Et₃N-salt, 9.0 mg, 5%). ¹H NMR (600 MHz, D₂O): δ 7.46 (dt, J=7.2, 1.3 Hz, 2H), 7.42 (tq, J=6.4, 1.1 Hz, 2H) 7.40-7.36 (m, 1H), 7.27 (dd, J=8.4, 1.1 Hz, 1H), 6.41 (dd, J=8.5, 1.2 Hz, 1H), 5.92 (dd, J=5.4, 1.2 Hz, 1H), 5.00 (s, 2H), 4.34-4.29 (m, 2H), 4.19 (q, J=3.2 Hz, 1H), 4.12-4.08 (m, 2H), 3.18 (q, J=7.3 Hz, 12H), 2.16 (t, J=18.9 Hz, 2H), 1.26 (t, J=7.3 Hz, 18H). ¹³C NMR (150 MHz, D₂O): δ 153.0, 151.1, 137.0, 132.7, 129.1 (2C), 128.7 (2C), 128.5, 94.3, 88.3, 83.2 (d, J=7.4 Hz), 75.6, 72.9, 70.0, 63.7 (d, J=4.6 Hz), 46.7 (6C), 27.4 (t, J=124.4 Hz), 8.3 (6C). ³¹P NMR (160 MHz, D₂O): δ 18.3, 15.0. MS (ESI, m/z) 534.1 [M+H]⁻; ESI-HRMS calcd. m/z for C₁₉H₂₆N₃O₁₁P₂ 534.1048, found 534.1074 [M−H]⁻. HPLC purity >99% (Rₜ=11.9 min, Method HPLC-B).

3-Deazauridine-5′-O-[(phosphonomethyl)phosphonic acid] (10). Method A. The product was obtained as colorless solid after lyophilization (2 eq Et₃N-salt, 19 mg, 15%). ¹H NMR (400 MHz, D₂O): δ 7.92 (d, J=7.8 Hz, 1H), 6.31 (d, J=7.6 Hz, 1H), 6.19 (d, J=4.3 Hz, 1H), 5.87 (d, J=2.6 Hz, 1H), 4.37 (dt, J=18.0, 5.1 Hz, 2H), 4.33-4.27 (m, 1H), 4.27-4.09 (m, 2H), 3.21 (q, J=7.3 Hz, 12H), 2.20 (t, J=19.7 Hz, 2H), 1.28 (t, J=7.3 Hz, 18H). ¹³C NMR (100 MHz, D₂O): δ 169.7, 165.7, 134.7, 104.2, 88.4, 82.9 (d, J=7.8 Hz), 74.7, 69.4, 63.2, 46.7 (6C), 27.5 (t, J=124.3 Hz), 8.3 (6C). ³¹P NMR (160 MHz, D₂O): δ 18.3, 14.7. MS (ESI, m/z) 400.0 [M−H]⁻; ESI-HRMS calcd. m/z for C₁₁H₁₆NO₁₁IP₂ 400.0199, found 400.0203 [M−H]⁻. HPLC purity 96% (Rₜ=9.4 min Method HPLC-C).

(S)-Methanocarbauridine-5′-O-[(phosphonomethyl)phosphonic acid] (11). Method A. The product was obtained as colorless solid after lyophilization (2 eq Et₃N-salt, 1.4 mg, 5%). ¹H NMR (400 MHz, D₂O): δ 7.76 (d, J=7.8 Hz, 1H), 5.83 (d, J=7.8 Hz, 1H), 4.67 (d, J=6.3 Hz, 1H), 4.12 (d, J=6.5 Hz, 1H), 4.10-3.98 (m, 2H), 3.21 (q, J=7.3 Hz, 12H), 2.35 (s, 1H), 2.22-2.02 (m, 2H), 1.86 (dd, J=9.5, 4.8 Hz, 1H), 1.68 (t, J=5.5 Hz, 1H), 1.28 (t, J=7.3 Hz, 18H). ¹³C NMR (100 MHz, D₂O): δ 166.9, 152.7, 148.2, 101.8, 75.0, 71.9, 65.4, 51.6, 48.3, 46.8 (6C), 24.8, 15.7, 8.3 (6C). ³¹P NMR (160 MHz, D₂O): δ 19.3, 14.1. MS (ESI, m/z) 411.0 [M−H]⁻; ESI-HRMS calcd. m/z for C₁₂H₁₇N₂O₁₀P₂ 411.0358, found 411.0363 [M−H]⁻. HPLC purity 96% (Rₜ=2.9 min, Method HPLC-A).

### Example 2

This examples demonstrates the synthesis of compounds in accordance with an embodiment of the invention.

HPLC: Method D. For the determination of HPLC purity of the nucleosides a different analytical HPLC method was used. Method D: Eluent: demineralized H₂O with 0.05% (v/v) trifluoroacetic acid —CH₃CN with 0.05% (v/v) trifluoroacetic acid 90:10 for 0 to 4 min, then from 90:10 to 0:100 in 25 min, 0:100 for 2 min, then from 0:100 to 90:10 in 0.5 min. Column: LiChrospher™ 60 RP-select B (5.0 μm), 250×4 mm; Merck KGaA. Peaks were detected by UV absorption (210 nm) using a diode array detector.

Procedure for the synthesis of compounds 3c-e and 8h-I (46). A suspension of uridine (3a, 1.0 eq) or N⁴[O-(benzyloxy)]-cytidine (8b, 1.0 eq), K₂CO₃ (1.7 eq) and corresponding alkyl halide (1.5 eq) was stirred in DMF/acetone (1:1) at 55-60° C. for 3 d. The mixture was cooled to room temperature, filtered and the residue was washed with acetone and purified by flash column chromatography (MeOH/DCM 1:20 to 1:10).

3-Ethyluridine (3c) (42), The product was obtained as colorless solid after flash column chromatography (748 mg, 67%). ¹H NMR (400 MHz, CD₃OD): δ 8.01 (d, J=8.1 Hz, 1H), 5.92 (d, J=4.0 Hz, 1H), 5.76 (d, J=8.1 Hz, 1H), 4.23-4.11 (m, 2H), 4.01 (dt, J=4.7, 3.0 Hz, 1H), 3.97 (q, J=7.1, 2H), 3.85 (dd, J=12.3, 2.7 Hz, 1H), 3.74 (dd, J=12.2, 3.1 Hz, 1H), 1.19 (t, J=7.1 Hz, 3H). ¹³C NMR (100 MHz, CD₃OD): δ 164.8, 152.4, 140.8, 102.1, 91.6, 86.3, 75.9, 71.1, 62.1, 37.2, 13.0. APCI-HRMS calcd. m/z for C₁₁H₁₇N₂O₆ 273.1081, found 273.1072 [M+H]+. HPLC purity 81% (Rₜ=2.2 min, Method HPLC-C).

3-Propyluridine (3d) (42). The product was obtained as colorless solid after flash column chromatography (902 mg, 75%). ¹H NMR (400 MHz, CD₃OD): δ 8.02 (d, J=8.0 Hz, 1H), 5.92 (d, J=4.2 Hz, 1H), 5.76 (d, J=8.1 Hz, 1H), 4.18-4.13 (m, 2H), 4.01 (dt, J=4.9, 2.9 Hz, 1H), 3.90-3.83 (m, 3H), 3.74 (dd, J=12.3, 3.1 Hz, 1H), 1.63 (h, J=7.5 Hz, 2H), 0.93 (t, J=7.5 Hz, 3H). ¹³C NMR (100 MHz, CD₃OD): δ 165.0, 152.6, 140.8, 102.0, 91.6, 86.2, 75.9, 71.1, 62.1, 43.6, 21.8, 11.5. APCI-HRMS calcd. m/z for C₁₂H₁₉N₂O₆ 287.1238, found 287.1238 [M+H]+. HPLC purity >99% (Rₜ=5.2 min, Method HPLC-C).

3-Benzyluridine (3e) (42). The product was obtained as colorless solid after flash column chromatography (942 mg, 69%). ¹H NMR (400 MHz, CD₃OD): δ 8.04 (d, J=8.1 Hz, 1H), 7.40-7.33 (m, 2H), 7.31-7.26 (m, 2H), 7.26-7.20 (m, 1H), 5.92 (d, J=3.8 Hz, 1H), 5.80 (d, J=8.1 Hz, 1H), 5.09 (s, 2H), 4.19-4.08 (m, 2H), 4.01 (dt, J=4.6, 2.9 Hz, 1H), 3.85 (dd, J=12.3, 2.7 Hz, 1H), 3.73 (dd, J=12.3, 3.1 Hz, 1H). ¹³C NMR (100 MHz, CD₃OD): δ 164.9, 152.6, 141.0, 138.2, 129.4 (2C), 129.3 (2C), 128.5, 102.0, 91.7, 86.3, 75.9, 71.1, 62.1, 45.1. APCI-HRMS calcd. m/z for C₁₆H₁₉N₂O₆ 335.1238, found 335.1236 [M+H]⁺. HPLC purity 92% (Rₜ=2.7 min, Method HPLC-C).

N⁴-[O-(Benzyloxy)]-3-methylcytidine (8h). The product was obtained as colorless solid after flash column chromatography (112 mg, 56%). ¹H NMR (400 MHz, CD₃OD): δ 7.38-7.24 (m, 6H), 6.24 (d, J=8.3 Hz, 1H), 5.87 (d, J=4.8 Hz, 1H), 4.98 (s, 2H), 4.15-4.08 (m, 3H), 3.94 (td, J=3.7, 3.1 Hz, 1H), 3.79 (dd, J=12.2, 2.9 Hz, 1H), 3.69 (dd, J=12.2, 3.5 Hz, 1H), 3.19 (s, 3H). ¹³C NMR (100 MHz, CD₃OD): δ 152.0, 151.4, 139.6, 133.2, 129.2(4) (2C), 129.2(5) (2C), 128.7, 93.9, 90.8, 86.1, 76.9, 75.0, 71.5, 62.7, 29.3. APCI-HRMS calcd. m/z for C₁₇H₂₂N₃O₆ 364.1503, found 364.1497 [M+H]+. HPLC purity 87% (Rₜ=7.9 min, Method HPLC-D).

N⁴-[O-(Benzyloxy)]-3-ethylcytidine (8i). The product was obtained as colorless solid after flash column chromatography (100 mg, 46%). ¹H NMR (400 MHz, CD₃OD): δ 7.37-7.29 (m, 4H), 7.28-7.24 (m, 1H), 7.23 (d, J=8.4 Hz, 1H), 6.21 (d, J=8.4 Hz, 1H), 5.87 (d, J=5.1 Hz, 1H), 4.97 (s, 2H), 4.13-4.09 (m, 2H), 3.94 (q, J=3.5 Hz, 1H), 3.88 (q, J=7.0 Hz, 1H), 3.78 (dd, J=12.2, 2.9 Hz, 1H),), 3.69 (dd, J=12.2, 3.5 Hz, 1H), 1.11), 3.78 (t, J=7.0 Hz, 1H). ¹³C NMR (100 MHz, CD₃OD): δ 151.6, 150.1, 139.7, 133.1, 129.3 (2C), 129.2 (2C), 128.7, 94.1, 90.6, 86.0, 76.9, 74.9, 71.6, 62.7, 38.2, 11.9. APCI-HRMS calcd. m/z for C₁₈H₂₄N₃O₆ 378.1660, found 378.1665 [M+H]+. HPLC purity 95% (Rₜ=9.2 min, Method HPLC-D).

### Example 3

This examples demonstrates the synthesis of compounds in accordance with an embodiment of the invention.

Procedure for the synthesis of compounds 8c-g (43). To a solution of hydroxylamine hydrochloride (2.0 eq) in pyridine (10 mL), cytidine-derivative (1.0 eq) was added and the suspension was stirred at 80° C. for overnight. The mixture was concentrated in vacuo and purified by flash column chromatography (CH₃OH/DCM 1:10).

N⁴-[O-(Benzyloxy)]-2′deoxycytidine (8c). The product was obtained as colorless solid after flash column chromatography (44).

N⁴-[O-(4-Trifluoromethylbenzyloxy)]-cytidine (8d). The product was obtained as colorless solid after flash column chromatography (423 mg, 77%). ¹H NMR (400 MHz, aceton-d6): δ 9.07 (bs, 1H), 7.72-7.61 (m, 4H), 7.30-7.24 (m, 1H), 5.91-5.85 (m, 1H), 5.52 (d, J=8.2 Hz, 1H), 5.10 (s, 2H), 4.65 (bs, 1H), 4.40-4.18 (m, 4H), 3.98 (s, 1H), 3.83-3.69 (m, 2H). ESI-HRMS calcd. m/z for C₁₇H₁₉F₃N₃O₆ 418.1226, found 418.1228 [M+H^{]}+.

N⁴-[O-(Naphth-2-yl-methyloxy)]-cytidine (8e). The product was obtained as colorless solid after flash column chromatography (373 mg, 79%). ¹H NMR (400 MHz, CD₃OD): δ 7.88-7.79 (m, 4H), 7.52 (d, J=9.4 Hz, 1H), 7.49-7.43 (m, 2H), 7.20 (d, J=8.2 Hz, 1H), 5.86 (d, J=5.4 Hz, 1H), 5.56 (d, J=8.2 Hz, 1H), 5.18 (s, 2H), 4.19-4.05 (m, 2H), 3.93 (q, J=3.3 Hz, 1H), 3.77 (dd, J=12.2, 2.9 Hz, 1H), 3.68 (dd, J=12.2, 3.4 Hz, 1H). APCI-HRMS calcd. m/z for C₂₀H₂₂N₃O₆ 400.1503, found 400.1534 [M+H]+.

N⁴-[O-(Benzyloxy)]-5-methylcytidine (80. The product was obtained as colorless solid after flash column chromatography. ¹H NMR (400 MHz, CD₃OD): δ 7.41-7.36 (m, 2H), 7.35-7.30 (m, 2H), 7.30-7.24 (m, 1H), 7.02 (d, J=1.4 Hz, 1H), 5.84 (d, J=5.3 Hz, 1H), 5.05 (s, 2H), 4.16-4.10 (m, 2H), 3.93 (q, J=3.3 Hz, 1H), 3.79 (dd, J=12.2, 2.9 Hz, 1H), 3.69 (dd, J=12.2, 3.4 Hz, 1H), 1.77 (d, J=1.4 Hz, 3H). ¹³C NMR (100 MHz, CD₃OD): δ 151.6, 146.4, 139.4, 129.3 (2C), 129.2 (2C), 129.1, 128.8, 107.9, 89.6, 86.1, 76.8, 74.6, 71.6, 62.7, 12.9. APCI-HRMS calcd. m/z for C₁₇H₂₂N₃O₆ 364.1503, found 364.1538 [M+H]⁺.

N⁴-[O-(Benzyloxy)]-5-fluorocytidine (8g). The product was obtained as colorless solid after flash column chromatography (472 mg, 83%). ¹H NMR (600 MHz, CD₃OD): δ 7.51 (d, J=7.9 Hz, 1H), 7.41-7.26 (m, 5H), 5.86 (dd, J=5.0, 1.9 Hz, 1H), 5.08 (s, 2H), 4.13-4.08 (m, 2H), 3.96-3.93 (m, 1H), 3.79 (dd, J=12.1, 2.7 Hz, 1H), 3.70 (dd, J=12.1, 2.9 Hz, 1H). ¹³C NMR (150 MHz, CD₃OD): δ 150.3, 140.3 (d, J=21.6 Hz), 139.1(2) (d, J=234.6 Hz), 139.0(5), 129.3(4) (2C), 129.2(7) (2C), 129.0, 117.1 (d, J=35.5 Hz), 89.8, 86.3, 77.2, 75.0, 71.7, 62.5. APCI-HRMS calcd. m/z for C₁₆H₁₆FN₃O₆ 368.1252, found 368.1249 [M+H]+. HPLC purity >99% (Rₜ=6.2 min, Method HPLC-C).

### Example 4

This example demonstrates the synthesis of compounds in accordance with an embodiment of the invention.

Synthesis of 1-(bromomethyl)-4-ethynylbenzene (124b). To a solution of 4-ethynylbenzyl alcohol (124a, 200 mg, 1.5 mmol) in anhydrous THF (7.0 mL) at 0° C., was added phosphorus tribromide (172 μL, 1.8 mmol) dropwise and the mixture stirred at room temperature for 12 h. Solvent was removed under reduced pressure and the resulting yellow oil was purified by silica gel column (hexane) to afford the pure compound as a colorless oil (273 mg, 93%).

¹H NMR (MeOD, 400 Hz) δ 7.55 (d, J=8.0 Hz, 2H), 7.43 (d, J=8.2 Hz, 2H), 4.80 (s, 2H), 3.57 (s, 1H).

### General Procedure for the Synthesis of Substituted O-benzylhydroxylamine hydrochloride (215-135)

Compound 25-35 were synthesized following the reported procedure¹ starting from the corresponding benzyl bromide (when commercially available) or alcohol (e.g. 124a).

O-2-Chlorobenzylhydroxylamine hydrochloride (125). The product was obtained as a white solid (120 mg) in 63% yield. ¹H NMR (MeOD, 400 Hz) δ 7.35-7.55 (m, 4H), 5.20 (s, 2H).

O-3-Chlorobenzylhydroxylamine hydrochloride (126). The product was obtained as a white solid (105 mg) in 74% yield. ¹H NMR (MeOD, 400 Hz) δ 7.36-7.47 (m, 4H), 5.02 (s, 2H).

O-3-(Trifluoromethyl)benzylhydroxylamine hydrochloride (127). The product was obtained as a white solid (130 mg) in 68% yield. ¹H NMR (MeOD, 400 Hz) δ 7.61-7.78 (m, 4H), 5.12 (s, 2H).

O-4-Fluorobenzylhydroxylamine hydrochloride (128). The product was obtained as a white solid (150 mg) in 75% yield. ¹H NMR (MeOD, 400 Hz) δ 7.48 (dd, J=5.4, 8.4 Hz, 2H), 7.16 (t, J=8.7 Hz, 2H), 5.03 (s, 2H).

O-4-Chlorobenzylhydroxylamine hydrochloride (129). The product was obtained as a white solid (100 mg) in 70% yield. ¹H NMR (MeOD, 400 Hz) δ 7.45-7.59 (m, 4H), 5.04 (s, 2H).

O-4-Bromobenzylhydroxylamine hydrochloride (130). The product was obtained as a white solid (120 mg) in 74% yield. ¹H NMR (MeOD, 400 Hz) δ 7.60 (d, J=8.3 Hz, 2H), 7.38 (d, J=8.2 Hz, 2H), 5.02 (s, 2H).

O-4-Iodobenzylhydroxylamine hydrochloride (131). The product was obtained as a white solid (124 mg) in 64% yield. ¹H NMR (MeOD, 400 Hz) δ 7.82 (d, J=8.3 Hz, 2H), 7.22 (d, J=8.2 Hz, 2H), 4.98 (s, 2H).

O-4-(Trifluoromethyl)benzylhydroxylamine hydrochloride (132). The product was obtained as a white solid (145 mg) in 76% yield. ¹H NMR (MeOD, 400 Hz) δ 7.95 (d, J=8.0 Hz, 2H), 7.65 (d, J=8.0 Hz, 2H), 5.12 (s, 2H).

O-4-(Pentafluorosulfanyl)benzylhydroxylamine hydrochloride (133). The product was obtained as a white solid (114 mg) in 59% yield. ¹H NMR (MeOD, 400 Hz) δ 7.90 (d, J=8.7 Hz, 2H), 7.60 (d, J=8.3 Hz, 2H), 5.10 (s, 2H).

O-4-Ethynylbenzylhydroxylamine hydrochloride (134). The product was obtained as a white solid (160 mg) in 62% yield, starting from compound 124b. ¹H NMR (MeOD, 400 Hz) δ 7.55 (d, J=8.0 Hz, 2H), 7.43 (d, J=8.2 Hz, 2H), 5.05 (s, 2H), 3.55 (s, 1H).

O-4-(Methylbenzoate)benzylhydroxylamine hydrochloride (135). The product was obtained as a white solid (123 mg) in 64% yield. ¹H NMR (MeOD, 400 Hz) δ 8.09 (d, J=8.2 Hz, 2H), 7.56 (d, J=8.1 Hz, 2H), 5.11 (s, 2H), 3.92 (s, 3H).

Synthesis of 3-methylcytidine (136). To a solution of cytosine (1.0 g, 4.1 mmol) in N,N-dimethylacetamide (Dmac, 25 mL) was added methyl iodide (7.7 mL, 12.3 mmol) and the mixture stirred at room temperature for 4 h. Solvent was removed under reduced pressure and the resulting crude mixture resuspended in dichloromethane with the formation of a white precipitate that was filtered, obtaining 1.0 g of the pure compound (95% yield). ¹H NMR (MeOD, 400 Hz) δ 8.5 (d, J=7.8 Hz, 1H), 6.15 (d, J=7.9 Hz, 1H), 5.87 (d, J=2.6 Hz, 1H), 4.14-4.21 (m, 2H), 4.08 (t, J=3.1 Hz, 1H), 3.95 (dd, J=2.12, 12.4 Hz, 1H), 3.79 (dd, J=2.12, 12.4 Hz, 1H), 3.49 (s, 3H). HRMS calcd C₁₀H₁₇N₃O₅ (M+H)⁺: 258.1087; found 258.1090.

### General Procedure for the Synthesis of Compounds 137-147

A solution of compound 136 (N³-methylcytidine, 0.8 mmol) and the desired O-benzylhydroxylamine (125-135, 1.6 mmol) in dry pyridine was stirred at 80° C. for 12 h. Then the reaction mixture was concentrated under reduced pressure and purified by silica gel column.

N⁴-(2-Chlorobenzyloxy)-3-methylcytidine (137). Compound 137 was synthesized following the procedure above starting from O-(2-chlorobenzyl)hydroxylamine hydrochloride. The crude was purified by silica gel column (methanol/dichloromethane 8:92) to obtain 96 mg of a white solid (97% yield). ¹H NMR (MeOD, 400 Hz) δ 7.45-7.50 (m, 1H), 7.40-7.43 (m, 1H), 7.25-7.35 (m, 3H), 6.30 (d, J=8.3 Hz, 1H), 5.89 (d, J=4.8 Hz, 1H), 5.11 (s, 2H), 4.10-4.17 (m, 2H), 3.95 (t, J=3.3 Hz, 1H), 3.80 (dd, J=2.7, 12.1 Hz, 1H), 3.70 (dd, J=2.7, 12.1 Hz, 1H), 3.18 (s, 3H). HRMS calcd C₁₇H₂₁C₁N₃O₆ (M+H)⁺: 398.1119; found 398.1121.

N⁴-(3-Chlorobenzyloxy)-3-methylcytidine (138). Compound 138 was synthesized following the procedure above starting from O-(3-chlorobenzyl)hydroxylamine hydrochloride. The crude was purified by silica gel column (methanol/dichloromethane 5:95) to obtain 66 mg of a white solid (74% yield). ¹H NMR (MeOD, 400 Hz) δ 7.38 (s, 1H), 7.25-7.35 (m, 4H), 6.26 (d, J=8.3 Hz, 1H), 5.89 (d, J=4.8 Hz, 1H), 5.00 (s, 2H), 4.10-4.16 (m, 2H), 3.95 (t, J=3.3 Hz, 1H), 3.80 (dd, J=2.7, 12.1 Hz, 1H), 3.70 (dd, J=2.7, 12.1 Hz, 1H), 3.18 (s, 3H). HRMS calcd C₁₇H₂₁C₁N₃O₆ (M+H)+: 398.1178; found 398.1121.

N⁴-(3-Trifluoromethylbenzyloxy)-3-methylcytidine (139). Compound 139 was synthesized following the procedure above starting from O-(3-trifluoromethylbenzyl)hydroxylamine hydrochloride. The crude was purified by silica gel column (methanol/dichloromethane 5:95) to obtain 29 mg of a white solid (59% yield). ¹H NMR (MeOD, 400 Hz) δ 7.50-7.65 (m, 5H), 7.31 (d, J=8.4 Hz, 1H), 6.23 (d, J=8.3 Hz, 1H), 5.05 (s, 2H), 4.10-4.16 (m, 2H), 3.95 (t, J=3.2 Hz, 1H), 3.80 (dd, J=2.7, 12.1 Hz, 1H), 3.68 (dd, J=2.7, 12.1 Hz, 1H), 3.15 (s, 3H). HRMS calcd C₁₈H₂₁F₃N₃O₆ (M+H)⁺: 431.1298; found 431.1304.

N⁴-(4-Fluorobenzyloxy)-3-methylcytidine (140). Compound 140 was synthesized following the procedure above starting from O-(4-fluorobenzyl)hydroxylamine hydrochloride. The crude was purified by silica gel column (methanol/dichloromethane 5:95) to obtain 56 mg of a white solid (35% yield). ¹H NMR (MeOD, 400 Hz) δ 7.32-7.41 (m, 2H), 7.29 (d, J=8.4 Hz, 1H), 7.00-7.10 (m, 2H), 6.21 (d, J=8.2 Hz, 1H), 5.85 (d, J=4.6 Hz), 4.95 (s, 2H), 4.05-4.15 (m, 2H), 3.95 (t, J=3.2 Hz, 1H), 3.75 (dd, J=2.7, 12.1 Hz, 1H), 3.68 (dd, J=2.7, 12.1 Hz, 1H), 3.16 (s, 3H). HRMS calcd C₁₇H₂₁FN₃O₆(M+H)⁺: 382.1409; found 382.1414.

N⁴-(4-Chlorobenzyloxy)-3-methylcytidine (141). Compound 141 was synthesized following the procedure above starting from O-(4-chlorobenzyl)hydroxylamine hydrochloride. The crude was purified by silica gel column (methanol/dichloromethane 5:95) to obtain 96 mg of a white solid (97% yield). ¹H NMR (MeOD, 400 Hz) δ 7.33-7.36 (m, 4H), 7.30 (d, J=8.4 Hz, 1H), 6.21 (d, J=8.2 Hz, 1H), 5.88 (d, J=4.6 Hz, 1H), 4.98 (s, 2H), 4.08-4.15 (m, 2H), 3.95 (t, J=3.2 Hz, 1H), 3.78 (dd, J=2.7, 12.1 Hz, 1H), 3.69 (dd, J=2.7, 12.1 Hz, 1H), 3.19 (s, 3H). HRMS calcd C₁₇H₂₁C₁N₃O₆ (M+H)⁺: 397.1041; found 397.1050.

N⁴-(4-Bromobenzyloxy)-3-methylcytidine (142). Compound 142 was synthesized following the procedure above starting from O-(4-bromobenzyl)hydroxylamine hydrochloride. The crude was purified by silica gel column (methanol/dichloromethane 6:94) to obtain 40 mg of a white solid (39% yield). ¹H NMR (MeOD, 400 Hz) δ 7.49 (d, J=8.3 Hz, 2H), 7.26-7.32 (m, 3H), 6.22 (d, J=8.2 Hz, 1H), 5.88 (d, J=4.6 Hz, 1H), 4.98 (s, 2H), 4.08-4.15 (m, 2H), 3.95 (t, J=3.2 Hz, 1H), 3.80 (dd, J=2.7, 12.1 Hz, 1H), 3.70 (dd, J=2.7, 12.1 Hz, 1H), 3.17 (s, 3H). HRMS calcd C₁₇H₂₁BrN₃O₆(M+H)⁺: 442.0614; found 442.0614.

N⁴-(4-Iodobenzyloxy)-3-methylcytidine (143). Compound 143 was synthesized following the procedure above starting from O-(4-iodobenzyl)hydroxylamine hydrochloride. The crude was purified by silica gel column (methanol/dichloromethane 5:95) to obtain 108 mg of a white solid (92% yield). ¹H NMR (MeOD, 400 Hz) δ 7.30 (d, J=8.3 Hz, 2H), 7.26-7.32 (m, 3H), 6.22 (d, J=8.2 Hz, 1H), 5.87 (d, J=4.6 Hz, 1H), 4.98 (s, 2H), 4.08-4.15 (m, 2H), 3.94 (t, J=3.2 Hz, 1H), 3.78 (dd, J=2.7, 12.1 Hz, 1H), 3.69 (dd, J=2.7, 12.1 Hz, 1H), 3.17 (s, 3H). HRMS calcd C₁₇H₂₁IN₃O₆ (M+H)⁺: 490.0471; found 490.0475.

N⁴-(4-Trifluorobenzyloxy)-3-methylcytidine (144). Compound 144 was synthesized following the procedure above starting from O-(4-trifluoromethylbenzyl)hydroxylamine hydrochloride. The crude was purified by silica gel column (methanol/dichloromethane 5:95) to obtain 111 mg of a white solid (83% yield). ¹H NMR (MeOD, 400 Hz) δ 7.64 (d, J=8.0 Hz, 2H), 7.53 (d, J=8.0, 2H), 7.32 (d, J=8.3 Hz, 1H), 6.27 (d, J=8.2 Hz, 1H), 5.88 (d, J=4.6 Hz, 1H), 5.07 (s, 2H), 4.08-4.14 (m, 2H), 3.95 (t, J=3.2 Hz, 1H), 3.79 (dd, J=2.7, 12.1 Hz, 1H), 3.70 (dd, J=2.7, 12.1 Hz, 1H), 3.16 (s, 3H). HRMS calcd C₁₈H₂₁F₃N₃O₆ (M+H)⁺: 432.1375; found 432.1382.

N⁴-(4-(Pentafluorosulfanyl)benzyloxy)-3-methylcytidine (145). Compound 145 was synthesized following the procedure above starting from O-(4-pentafluorosulfanylbenzyl)hydroxylamine hydrochloride. The crude was purified by silica gel column (methanol/dichloromethane 2:98) to obtain 68 mg of a white solid (69% yield). ¹H NMR (MeOD, 400 Hz) δ 7.78 (d, J=8.6 Hz, 2H), 7.51 (d, J=8.2, 2H), 7.32 (d, J=8.3 Hz, 1H), 6.26 (d, J=8.3 Hz, 1H), 5.86 (d, J=4.6 Hz, 1H), 5.07 (s, 2H), 4.11-4.16 (m, 2H), 3.95 (t, J=3.2 Hz, 1H), 3.79 (dd, J=2.7, 12.1 Hz, 1H), 3.71 (dd, J=2.7, 12.1 Hz, 1H), 3.16 (s, 3H). HRMS calcd C₁₇H₂₁F₅N₃O₆S (M+H)⁺: 490.1071; found 490.1071.

N⁴-(4-Ethynylbenzyloxy)-3-methylcytidine (146). Compound 146 was synthesized following the procedure above starting from O-(4-ethynylbenzyl)hydroxylamine hydrochloride. The crude was purified by silica gel column (methanol/dichloromethane 5:95) to obtain 91 mg of a white solid (54% yield). ¹H NMR (MeOD, 400 Hz) δ 7.45 (d, J=8.0 Hz, 2H), 7.25-7.36 (m, 3H), 6.23 (d, J=8.3 Hz, 1H), 5.89 (d, J=4.6 Hz, 1H), 4.99 (s, 2H), 4.10-4.16 (m, 2H), 3.94 (t, J=3.2 Hz, 1H), 3.79 (dd, J=2.7, 12.1 Hz, 1H), 3.69 (dd, J=2.7, 12.1 Hz, 1H), 3.46 (s, 1H), 3.16 (s, 3H). HRMS calcd C₁₉H₂₂N₃O₆ (M+H)⁺: 388.1509; found 388.1509.

Methyl 4-(((((Z)-1-((2R,3R,4S,5R)-3,4-dihydroxy-5-(hydroxymethyl)tetrahydrofuran-2-yl)-3-methyl-2-oxo-2,3-dihydropyrimidin-4(1H)-ylidene)amino)oxy)methyl)benzoate (147). Compound 147 was synthesized following the procedure above starting from methyl 4-((aminooxy)methyl)benzoate hydrochloride. The crude was purified by silica gel column (methanol/dichloromethane 5:95) to obtain 26 mg of a white solid (48% yield). ¹H NMR (MeOD, 400 Hz) δ 7.79 (d, J=8.2 Hz, 2H), 7.46 (d, J=8.1 Hz, 2H), 7.31 (d, J=8.3 Hz, 1H), 6.26 (d, J=8.3 Hz, 1H), 5.86 (d, J=4.7 Hz, 1H), 5.05 (s, 2H), 4.10-4.16 (m, 2H), 3.94 (t, J=3.2 Hz, 1H), 3.89 (s, 3H), 3.79 (dd, J=2.7, 12.1 Hz, 1H), 3.69 (dd, J=2.7, 12.1 Hz, 1H), 3.16 (s, 3H). HRMS calcd C₁₉H₂₄N₃O₈ (M+H)+: 422.1558; found 422.1547.

Synthesis of compound 148 through hydrolysis of the methyl ester moiety. To a solution of compound 147 (84 mg, 0.2 mmol) in MeOH (2 mL), was added a 1N solution of NaOH (2 mL) and the reaction stirred at rt for 40 min. Glacial acetic acid was added until neutrality. Then, the mixture was diluted with water and extracted with ethyl acetate (3×15 mL). The combined organic layer was dried over sodium sulfate, filtered and evaporated to obtain 62 mg (77%) of pure compound as a white solid. ¹H NMR (MeOD, 400 Hz) δ 8.05 (d, J=8.1 Hz, 2H), 7.50 (d, J=8.0 Hz, 2H), 7.41 (d, J=8.3 Hz, 1H), 6.37 (d, J=8.3 Hz, 1H), 5.99 (d, J=4.7 Hz, 1H), 5.14 (s, 2H), 4.20-4.27 (m, 2H), 4.05 (d, J=3.2 Hz, 1H), 3.87-3.93 (m, 1H), 3.78-3.84 (m, 1H), 3.28 (s, 3H). HRMS calcd C₁₈H₂₂N₃O₈(M+H)⁺: 408.1407; found 408.1408.

Synthesis of compound 150 through hydrolysis of the acetylated intermediate formed by serendipity. To a solution of compound 148 (30 mg, 0.07 mmol, with traces of acetic acid), HATU (42 mg, 0.11 mmol) and DIPEA (19 μL, 0.11 mmol) in anhydrous DMF, methylamine hydrochloride (6.5 mg, 0.09 mmol) was added and the reaction stirred at rt for 12 h. Next, solvent was removed, and the crude purified by silica gel column (5% MeOH/DCM) to obtain an acetylated intermediate as a white solid (149). The latter was treated with methanolic ammonia for 2 h at rt to deblock the acetylated group and eventually recover the starting material. TLC analysis showed formation of a new compound that was obtained pure directly after removal of the solvent. NMR and LC-MS confirmed the formation of compound x (19 mg, 64%, 2-step yield). ¹H NMR (MeOD, 400 Hz) δ 7.85 (d, J=8.2 Hz, 2H), 7.46 (d, J=8.1 Hz, 2H), 7.30 (d, J=8.3 Hz, 1H), 6.27 (d, J=8.2 Hz, 1H), 5.88 (d, J=4.6 Hz, 1H), 5.06 (s, 2H), 4.10-4.16 (m, 2H), 3.95 (d, J=3.1 Hz, 1H), 3.77-3.82 (m, 1H), 3.67-3.73 (m, 1H), 3.18 (s, 3H). HRMS calcd C₁₈H₂₂N₄O₇(M+H)⁺: 407.1567; found 407.1567.

### General Procedure for the Synthesis of Derivatives 151 and 152 by Amination of the Methyl Ester 47

A solution of compound 47 (20 mg, 0.047 mmol and 50 mg, 0.119 mmol, respectively) in the required amine (ethylene diamine for compound 151 or 1,4-diaminobutine for compound 52, 2 mL) was stirred at rt for 2 h. LC-MS analysis showed full conversion to the amide product. Solvent was removed under reduced pressure and the so obtained crude mixture directly used for the next step.

### Synthesis of Compound 154 by Click-Reaction with 1-Azido-2-Fluoroethane

1-Azido-2-fluoroethane (153) has been synthesized following the reported procedure² starting from 1-chloro-2-fluoroethane.

To a solution of compound 146 (20 mg, 0.05 mmol) and 153 (4.5 mg, 0.05 mmol) in THF/H₂O (1:1, 1.0 mL) were subsequently added sodium ascorbate (15.0 mg, 0.08 mmol) and CuSO₄×5 H₂O (6.2 mg, 0.03 mmol). The reaction was stirred at rt for 12 h. After removal of solvent, the crude mixture was purified by silica gel column (5% MeOH in DCM) to obtain the pure compound as a white solid (22.8 mg, 96%). ¹H NMR (MeOD, 400 Hz) δ 8.35 (s, 1H), 7.80 (d, J=8.1 Hz, 2H), 7.45 (d, J=8.1 Hz, 2H), 7.30 (d, J=8.3 Hz, 1H), 6.26 (d, J=8.3 Hz, 1H), 5.87 (d, J=4.7 Hz, 1H), 5.02 (s, 2H), 4.78-4.82 (m, 2H), 4.06-4.15 (m, 4H), 3.82-3.75 (m, 1H), 3.72-3.68 (m, 1H), 3.19 (s, 3H). HRMS calcd C₂₂H₃₀FN₆O₁₁P₂(M+H)⁺: 477.1898; found 477.1893.

(2R,3S,4R,5S)-1-(Acetoxymethyl)-4-(2,4-dioxo-3,4-dihydropyrimidin-1(2H)-yl)-1λ-bicyclo[3.1.0]hexane-2,3-diyl diacetate (156). Compound 155 was prepared following a reported procedure. A mixture of compound 155 (100 mg, 0.4 mmol), acetic anhydride (148 μL, 1.6 mmol), triethylamine (219 μL, 1.6 mmol) and DMAP (24 mg, 0.2 mmol) in acetonitrile (3 mL) was stirred at rt for 2 h. Solvent was removed under reduced pressure and the residue purified by silica gel column (50% ethyl acetate in hexane) to obtain 100.0 mg of the pure compound as a white solid (67%). ¹H NMR (CDCl₃, 400 Hz) δ 9.95 (s, 1H), 7.36 (d, J=7.9 Hz, 1H), 5.67 (dd, J=7.7, 12.9 Hz, 2H), 5.15 (d, J=7.2 Hz, 1H), 4.58 (s, 1H), 4.34 (d, J=12.0 Hz, 1H), 3.96 (d, J=12.0 Hz, 1H), 2.04 (s, 3H), 1.99 (s, 6H), 1.47 (dd, J=3.8, 8.4 Hz, 1H), 1.21 (dd, J=5.3, 9.6 Hz, 2H), 0.95 (t, J=7.0 Hz, 1H). HRMS calcd C₁₇H₂₂N₂O₈(M+H)⁺: 382.1381; found 382.1384.

(2R,3S,4R,5S)-1-(Acetoxymethyl)-4-((Z)-4-(((4-chlorobenzyl)oxy)imino)-2-oxo-3,4-dihydropyrimidin-1(2H)-yl)-1λ⁵-bicyclo[3.1.0]hexane-2,3-diyl diacetate (157). Compound 57 was synthesized following the reported procedure¹ starting from compound 156 and O-4-chlorobenzylhydroxylamine hydrochloride (129). Purification was performed by silica gel column (50% Ethyl acetate in hexane) to obtain 36 mg of the pure product as a white powder (26% yield for 2 steps). ¹H NMR (CDCl₃, 400 Hz) δ 8.13 (s, 1H), 7.30 (dd, J=8.3, 19.6 Hz, 4H), 6.77 (d, J=8.1 Hz, 1H), 5.65 (d, J=6.3, 1H), 5.56 (dd, J=2.0, 8.1 Hz, 1H), 5.14 (d, J=7.1 Hz, 1H), 4.97 (s, 2H), 4.62 (s, 1H), 4.42 (d, J=12.0 Hz, 1H), 3.90 (d, J=12.0 Hz, 1H), 2.07 (s, 3H), 2.04 (s, 6H), 1.42 (dd, J=4.0, 8.7 Hz, 1H), 1.22-1.29 (m, 2H), 0.94 (t, J=6.8 Hz, 1H). HRMS calcd C₂₄H₂₈C₁N₃O₈(M+H)+: 520.1487; found 520.1481.

(2R,3S,4R,5S)-1-(Acetoxymethyl)-4-((Z)-4-(((4-chlorobenzyl)oxy)imino)-3-methyl-2-oxo-3,4-dihydropyrimidin-1(2H)-yl)-1λ⁵-bicyclo[3.1.0]hexane-2,3-diyl diacetate (158). To a mixture of compound 157 (36.0 mg, 0.07 mmol) and K₂CO₃ (15 mg, 0.10 mmol) in N,N-dimethylacetamide (Dmac, 2.0 mL) was added methyl iodide (13 μL, 0.20 mmol) and the mixture stirred at room temperature for 18 h. Solvent was removed under reduced pressure and the resulting crude mixture purified by silica gel column (50% ethyl acetate in hexane) to obtain 30.7 mg of the pure product (83%). ¹H NMR (CDCl₃, 400 Hz) δ 7.30 (s, 4H), 6.80 (d, J=8.2 Hz, 1H), 6.16 (d, J=8.2, 1H), 5.69 (d, J=7.2 Hz, 1H), 5.14 (d, J=7.2 Hz, 1H), 4.96 (s, 2H), 4.58 (s, 1H), 4.41 (d, J=12.0 Hz, 1H), 3.92 (d, J=12.0 Hz, 1H), 3.19 (s, 3H), 2.10 (s, 3H), 2.04 (s, 6H), 1.42 (dd, J=4.0, 8.7 Hz, 1H), 1.25 (t, J=4.9 Hz, 2H), 0.94 (t, J=7.3 Hz, 1H). HRMS calcd C₂₅H₃₀C₁N₃O₈(M+H)⁺: 534.1643; found 534.1647.

(Z)-4-(((4-Chlorobenzyl)oxy)imino)-1-((1S,2R,3S,4R)-3,4-dihydroxy-5-(hydroxymethyl)-5λ⁵-bicyclo[3.1.0]hexan-2-yl)-3-methyl-3,4-dihydropyrimidin-2(1H)-one (159). A solution of compound 158 (30.7 mg, 0.06 mmol) in NH₃ methanol solution (2.0 mL) was stirred at rt for 18 h. The solvent was removed, and the crude purified by silica gel column (5% methanol in dichloromethane) to obtain 21.0 mg of the pure product (91%). ¹H NMR (MeOD, 400 Hz) δ 7.38 (d, J=8.2 Hz, 1H), 7.32 (d, J=1.0 Hz, 4H), 6.17 (d, J=8.2, 1H), 4.95 (s, 2H), 4.65 (s, 1H), 4.55 (d, J=6.7 Hz, 1H), 4.16 (d, J=11.7 Hz, 1H), 3.75 (d, J=6.7 Hz, 1H), 3.23 (d, J=11.7 Hz, 1H), 3.17 (s, 3H), 1.26-1.33 (m, 2H), 0.64 (t, J=3.3 Hz, 1H). HRMS calcd C₁₉H₂₄C₁N₃O₅(M+H)⁺: 408.1326; found 408.1330.

### Synthesis of Compound 161 by Acylation of Cytidine

A mixture of cytidine (200 mg, 0.8 mmol), acetic anhydride (310 μL, 3.3 mmol), triethylamine (458 μL, 3.3 mmol) and DMAP (50 mg, 0.4 mmol) in acetonitrile (4 mL) was stirred at rt for 10 min with formation of both tri-O-acetyl-cytidine and tetra-O,N₄-acetyl-cytidine. Solvent was removed under reduced pressure and the two derivatives separated by silica gel column (10% methanol in dichlomethane). Next, the tetra-O,N⁴-acetyl-cytidine was dissolved in MeOH (3 mL) and heated at 105° C. for 3 h in a microwave reactor with full conversion of the latter to tri-O-acetyl-cytidine (purified with the same method as before). ⁵ Total yield for two steps 95% (289 mg).

### Synthesis of Tri-O-Acetyl-3-Methylcytidine (162)

Compound 162 was synthesized as reported above for the 3-methylcytidine, starting from compound 161. ¹H NMR (CDCl₃, 400 Hz) δ 7.4 (s, 1H), 7.05 (d, J=8.0 Hz, 1H), 6.00 (d, J=8.0 Hz, 1H), 5.95 (d, J=4.7 Hz, 1H), 5.27-5.36 (m, 2H), 4.29-4.33 (m, 3H), 3.44 (s, 3H), 2.12 (s, 3H), 2.08 (t, J=4.9 Hz, 6H). HRMS calcd C₁₆H₂₃N₃O₈(M+H)⁺: 384.1407; found 384.1410.

### Synthesis of Tri-O-acetyl-4-((benzylcarbamoyl)imino)-3-methylcytidine (613)

N-benzyl-1H-imidazole-1-carboxamide (160) was synthesized as reported.⁶ A solution of compound 162 (73 mg, 0.2 mmol), compound 160 (77 mg, 0.4 mmol) and triethylamine (27 μL, 0.2 mmol) in THF (3 mL) was stirred at reflux for 12 h. Solvent was removed under reduced pressure and the crude mixture purified by silica gel column (2% methanol in dichloromethane) to obtain the pure product as a white solid (72 mg, 72%). ¹H NMR (CDCl₃, 400 Hz) δ 7.26-7.33 (m, 5H), 7.13 (d, J=8.2 Hz, 1H), 6.78 (d, J=8.2 Hz, 1H), 6.00 (d, J=4.4 Hz, 1H), 5.27-5.36 (m, 2H), 4.45 (d, J=5.9 Hz, 2H), 4.32 (s, 3H), 3.33 (s, 3H), 2.12 (s, 3H), 2.09 (s, 3H), 2.08 (s, 3H). HRMS calcd C₂₄H₂₉ N₄O₉(M+H)⁺: 517.1935; found 517.1940.

### Synthesis of 4-((benzylcarbamoyl)imino)-3-methylcytidine (164)

A suspension of compound 163 (72 mg, 0.1 mmol) in methanolic ammonia solution (2 mL) was stirred at rt for 12 h. Solvent was removed, and the crude mixture purified by silica gel column (7% methanol in dichloromethane) to obtain the pure product as a white solid (47 mg, 87%). ¹H NMR (MeOD, 400 Hz) δ 7.72 (d, J=8.2, 1H), 7.22-7.33 (m, 5H), 6.18 (d, J=8.2 Hz, 1H), 5.90 (d, J=3.7 Hz, 1H), 4.38 (s, 2H), 4.12 (dd, J=4.4, 8.2 Hz, 2H), 3.98 (d, J=3.0 Hz, 1H), 3.83 (dd, J=2.8, 12.2 Hz, 1H), 3.71 (dd, J=2.8, 12.2 Hz, 1H), 3.35 (s, 3H). HRMS calcd C₁₈H₂₃N₄O₆(M+H)⁺: 391.1618; found 391.1616.

### General Procedure for the Synthesis of Nucleotides (105-123)

To a solution of methylenebis(phosphonic dichloride) (3 eq) in trimethylphosphate (3 mL) at 0° C. was added a solution of the desired compound (137-148, 150-152, 154, 155, 159, or 164, 1 eq) in trimethylphosphate (2 mL) at 0° C. The so obtained mixture was stirred at 0° C. for 3 h and then a sample was withdrawn for LC-MS to check the disappearance of the nucleoside. Then, the reaction was quenched with 7 mL of cold 1 M aqueous triethylammonium hydrogen carbonate buffer solution (pH=8.4-8.6) and stirred at room temperature for 1 h. Solvent was removed by lyophilization and the residue dissolved in water, purified first by ion-exchange chromatography and then by RP-HPLC to afford the pure product.

N⁴-(2-Chlorobenzyloxy)-3-methyleytidine-5′-α,β-methylenediphosphate triethylammonium salt (105). The product was obtained as a white solid after lyophilization (9.8 mg, 20%; RP-HPLC acetonitrile/10 mM triethylammonium acetate, 15/100 to 35/65 in 40 min @5 mL/min). ¹H NMR (D₂O, 400 Hz) δ 7.45-7.55 (m, 2H), 7.29-7.40 (m, 3H), 6.44 (d, J=8.3 Hz, 1H), 5.93 (d, J=4.5 Hz, 1H), 5.12 (s, 2H), 4.35 (s, 2H), 4.21 (s, 1H), 4.10 (s, 2H), 3.11-3.22 (m, 15H), 2.18 (t, J=19.7 Hz, 2H), 1.26 (t, J=7.1 Hz, 18H). ³¹P NMR (D₂O) δ 18.20, 14.21. HRMS calcd C₁₈H₂₃ClN₃O₁₁P₂ (M−H)⁻: 554.0496; found 554.0490. N⁴-(3-Chlorobenzyloxy)-3-methylcytidine-5′-α,β-methylenediphosphate triethylammonium salt (106). The product was obtained as a white solid after lyophilization (3.7 mg, 2.0%; RP-HPLC acetonitrile/10 mM triethylammonium acetate, 15/100 to 35/65 in 40 min @5 mL/min). ¹H NMR (D₂O, 400 Hz) δ 7.49 (s, 1H), 7.32-7.42 (m, 3H), 6.45 (d, J=8.4 Hz, 1H), 5.93 (d, J=4.9 Hz, 1H), 5.03 (s, 2H), 4.30-4.39 (m, 2H), 4.21 (s, 1H), 4.12 (s, 2H), 3.11-3.29 (m, 9H), 2.18 (t, J=19.7 Hz, 2H), 1.26 (t, J=6.7 Hz, 9H). ³¹P NMR (D₂O) δ 20.50, 12.50. HRMS calcd C₁₈H₂₃C₁N₃O₁₁P₂ (M−H)⁻: 554.0496; found 554.0503. N⁴-((3-Trifluoromethyl)benzyloxy)-3-methyleytidine-5′-α,β-methylenediphosphate triethylammonium salt (107). The product was obtained as a white solid after lyophilization (4.2 mg, 9.5%; RP-HPLC acetonitrile/10 mM triethylammonium acetate, 15/100 to 35/65 in 40 min @5 mL/min). ¹H NMR (D₂O, 400 Hz) δ 7.64 (s, 1H), 7.56 (d, J=7.6 Hz, 2H), 7.45 (t, J=7.6 Hz, 1H), 7.22 (d, J=8.3 Hz, 1H), 6.32 (d, J=8.3 Hz, 1H), 5.80 (d, J=4.7 Hz, 1H), 4.95 (s, 2H), 4.20 (d, J=4.8 Hz, 2H), 4.08 (s, 1H), 3.97 (s, 2H), 3.00-3.11 (m, 15H), 2.02 (t, J=19.7 Hz, 2H), 1.13 (t, J=7.1 Hz, 18H). ³¹P NMR (D₂O) δ 29.00, 24.00. ¹⁹F NMR (D₂O) δ −62.40. HRMS calcd C₁₉H₂₃F₃N₃O₁₁P₂ (M−H)⁻: 588.0760; found 588.0756.

N⁴-(4-Fluorobenzyloxy)-3-methyleytidine-5′-α,β-methylenediphosphate triethylammonium salt (108). The product was obtained as a white solid after lyophilization (12.9 mg, 27.0%; RP-HPLC acetonitrile/10 mM triethylammonium acetate, 15/100 to 35/65 in 40 min @5 mL/min). ¹H NMR (D₂O, 400 Hz) δ 7.36 (t, J=6.8, 2H), 7.22 (d, J=7.7 Hz, 1H), 7.02 (t, J=8.7 Hz, 2H), 6.30 (d, J=8.1 Hz, 1H), 5.81 (s, 1H), 4.84 (s, 2H), 4.16-4.27 (m, 2H), 3.91-4.09 (m, 3H), 3.00-3.12 (m, 15H), 2.00 (t, J=19.7 Hz, 2H), 1.13 (t, J=7.2 Hz, 18H). ³¹P NMR (D₂O) δ 21.00, 13.00. ¹⁹F NMR (D₂O) 6-75.50. HRMS calcd C₁₈H₂₃FN₃O₁₁P₂ (M−H)⁻: 538.0792; found 538.0789.

N⁴-(4-Chlorobenzyloxy)-3-methylcytidine-5′-α,β-methylenediphosphate triethylammonium salt (109). The product was obtained as a white solid after lyophilization (35.4 mg, 33.0%; RP-HPLC acetonitrile/10 mM triethylammonium acetate, 15/100 to 35/65 in 40 min @5 mL/min). ¹H NMR (D₂O, 400 Hz) δ 7.23 (s, 4H), 7.19 (d, J=8.4 Hz, 1H), 6.23 (d, J=8.3 Hz, 1H), 5.79 (d, J=5.0 Hz, 1H), 4.82 (s, 2H), 4.18-4.22 (m, 2H), 4.08 (d, J=1.8 Hz, 1H), 4.12 (s, 2H), 3.98-4.00 (m, 2H), 3.08 (dd, J=7.3, 14.6 Hz, 12H), 2.99 (s, 3H), 2.06 (t, J=19.7 Hz, 2H), 1.12 (t, J=7.3 Hz, 18H). ³¹P NMR (D₂O) δ 22.00, 15.00. HRMS calcd C₁₈H₂₃C₁N₃O₁₁P₂ (M−H)⁻: 554.0496; found 554.0501.

N⁴-(4-Bromobenzyloxy)-3-methyleytidine-5′-α,β-methylenediphosphate triethylammonium salt (110). The product was obtained as a white solid after lyophilization (3.5 mg, 20.0%; RP-HPLC acetonitrile/10 mM triethylammonium acetate, 15/100 to 35/65 in 40 min @5 mL/min). ¹H NMR (D₂O, 400 Hz) δ 7.50 (d, J=8.0, 2H), 7.28 (d, J=8.16 Hz, 3H), 6.36 (d, J=8.2 Hz, 1H), 5.82 (d, J=4.5 Hz, 1H), 4.91 (s, 2H), 4.31 (s, 1H), 4.22 (s, 1H), 4.00-4.12 (m, 3H), 3.10 (d, J=7.2 Hz, 21H), 1.95 (t, J=19.1 Hz, 2H), 1.20 (s, 27H). ³¹P NMR (D₂O) δ 21.00, 12.00. HRMS calcd C₁₈H₂₃BrN₃O₁₁P₂ (M−H)⁻: 597.9991; found 597.9988.

N⁴-(4-Iodobenzyloxy)-3-methylcytidine-5′-α,β-methylenediphosphate triethylammonium salt (111). The product was obtained as a white solid after lyophilization (8.2 mg, 22.1%; RP-HPLC acetonitrile/10 mM triethylammonium acetate, 15/100 to 35/65 in 40 min @5 mL/min). ¹H NMR (D₂O, 400 Hz) δ 7.69 (d, J=8.0, 2H), 7.24 (d, J=8.2 Hz, 1H), 7.12 (d, J=8.0, 2H), 6.32 (d, J=8.2 Hz, 1H), 5.82 (d, J=5.0 Hz, 1H), 4.88 (s, 2H), 4.25 (t, J=5.5 Hz, 2H), 4.12 (s, 1H), 4.01 (s, 2H), 3.10 (t, J=6.7 Hz, 6H), 3.03 (s, 3H), 2.05 (t, J=19.5 Hz, 2H), 1.18 (t, J=6.6 Hz, 9H). ³¹P NMR (D₂O) δ 23.00, 12.00. HRMS calcd C₁₈H₂₃IN₃O₁₁P₂ (M−H)⁻: 645.9852; found 645.9861.

N⁴-(4-(Trifluoromethyl)benzyloxy)-3-methyleytidine-5′-α,β-methylenediphosphate triethylammonium salt (112). The product was obtained as a white solid after lyophilization (1.4 mg, 10.0%; RP-HPLC acetonitrile/10 mM triethylammonium acetate, 15/100 to 35/65 in 40 min @5 mL/min). ¹H NMR (D₂O, 400 Hz) δ 7.58 (d, J=6.3, 2H), 7.43 (d, J=6.3 Hz, 2H), 7.22 (d, J=6.5, 1H), 6.32 (d, J=6.6 Hz, 1H), 5.79 (d, J=3.9 Hz, 1H), 4.94 (s, 2H), 4.23 (s, 1H), 4.17 (d, J=4.1 Hz, 1H), 4.00-4.05 (m, 2H), 3.92 (s, 1H), 3.02 (dd, J=5.8, 11.6 Hz, 6H), 2.98 (s, 3H), 1.91 (t, J=15.2 Hz, 2H), 1.10 (t, J=5.8 Hz, 9H). ³¹P NMR (D₂O) δ 22.00, 12.50. ¹⁹F NMR (D₂O) 6-62.00. HRMS calcd C₁₉H₂₃F₃N₃O₁₁P₂ (M−H)⁻: 588.0760; found 588.0755.

N⁴-(4-(Pentafluorosulfanyl)benzyloxy)-3-methyleytidine-5′-α,β-methylenediphosphate triethylammonium salt (113). The product was obtained as a white solid after lyophilization (8.0 mg, 23.0%; RP-HPLC acetonitrile/10 mM triethylammonium acetate, 15/100 to 35/65 in 40 min @5 mL/min). ¹H NMR (D₂O, 400 Hz) δ 7.75 (d, J=8.6, 2H), 7.46 (d, J=8.1 Hz, 2H), 7.23 (d, J=8.3, 1H), 6.37 (d, J=8.3 Hz, 1H), 5.82 (d, J=5.0 Hz, 1H), 4.99 (s, 2H), 4.22 (dd, J=4.4, 11.8 Hz, 2H), 4.11 (s, 1H), 4.00 (s, 2H), 3.09 (dd, J=7.3, 14.6 Hz, 6H), 3.02 (s, 3H), 2.05 (t, J=19.7 Hz, 2H), 1.14 (t, J=14.6 Hz, 9H). ³¹P NMR (D₂O) δ 22.00, 12.00. ¹⁹F NMR (D₂O) 6-75.55. HRMS calcd C₁₈H₂₃F₅N₃O₁₁P₂S (M−H)⁻: 646.0449; found 646.0452.

N⁴-(4-Ethynylbenzyloxy)-3-methyleytidine-5′-α,β-methylenediphosphate triethylammonium salt (114). The product was obtained as a white solid after lyophilization (15.2 mg, 35.0%; RP-HPLC acetonitrile/10 mM triethylammonium acetate, 15/100 to 35/65 in 40 min @5 mL/min). ¹H NMR (D₂O, 400 Hz) δ 7.42 (d, J=8.0, 2H), 7.31 (d, J=7.9 Hz, 2H), 7.22 (d, J=8.3, 1H), 6.31 (d, J=8.3 Hz, 1H), 5.79 (d, J=5.1 Hz, 1H), 4.89 (s, 2H), 4.18-4.24 (m, 2H), 4.07 (s, 1H), 4.02 (s, 2H), 3.92 (s, 1H), 3.41 (s, 1H), 3.01 (d, J=6.8 Hz, 18H), 3.00 (s, 3H), 2.02 (t, J=19.7 Hz, 2H), 1.14 (t, J=6.6 Hz, 27H). ³¹P NMR (D₂O) δ 19.00, 13.50. HRMS calcd C₂₀H₂₄N₃O₁₁P₂ (M−H)⁻: 544.0886; found 544.0884.

N⁴-(4-(Methoxycarbonyl)benzyloxy)-3-methyleytidine-5′-α,β-methylenediphosphate triethylammonium salt (115). The product was obtained as a white solid after lyophilization (38.7 mg, 32.0%; RP-HPLC acetonitrile/10 mM triethylammonium acetate, 15/100 to 30/70 in 40 min @5 mL/min). ¹H NMR (D₂O, 400 Hz) δ 7.80 (d, J=8.0, 2H), 7.33 (d, J=8.0 Hz, 2H), 7.20 (d, J=8.3, 1H), 6.30 (d, J=8.3 Hz, 1H), 5.77 (d, J=5.0 Hz, 1H), 4.91 (s, 2H), 4.21 (t, J=5.2 Hz, 2H), 4.07 (s, 1H), 3.98 (s, 2H), 3.74 (s, 3H), 3.09 (dd, J=7.1, 14.4 Hz, 12H), 2.92 (s, 3H), 2.04 (t, J=19.7 Hz, 2H), 1.13 (t, J=7.2 Hz, 18H).³¹P NMR (D₂O) δ 17.50, 14.00. HRMS calcd C₂₀H₂₆N₃O₁₃P₂ (M−H)⁻: 578.0941; found 578.0943.

N⁴-(4-(Carbonyl)benzyloxy)-3-methylcytidine-5′-α,β-methylenediphosphate triethylammonium salt (116). The product was obtained as a white solid after lyophilization (11.9 mg, 29.0%; RP-HPLC acetonitrile/10 mM triethylammonium acetate, 5/100 to 15/85 in 40 min @5 mL/min). ¹H NMR (D₂O, 400 Hz) δ 7.75 (d, J=7.8, 2H), 7.39 (d, J=7.7 Hz, 2H), 7.22 (d, J=8.2, 1H), 6.37 (d, J=8.2 Hz, 1H), 5.83 (s, 1H), 4.96 (s, 2H), 4.23 (s, 2H), 4.09 (s, 1H), 4.00 (s, 2H), 3.74 (s, 3H), 3.06 (dd, J=7.2, 14.3 Hz, 21H), 2.05 (t, J=19.6 Hz, 2H), 1.14 (t, J=7.2 Hz, 27H). ³¹P NMR (D₂O) δ 21.00, 15.40. HRMS calcd C₁₉H₂₄N₃O₁₃P₂ (M−H)⁻: 564.0784; found 564.0781.

N⁴-(4-(Carbamoyl)benzyloxy)-3-methyleytidine-5′-α,β-methylenediphosphate triethylammonium salt (117). The product was obtained as a white solid after lyophilization (1.6 mg, 9.6%; RP-HPLC acetonitrile/10 mM triethylammonium acetate, 5/100 to 15/85 in 40 min @5 mL/min). ¹H NMR (D₂O, 400 Hz) δ 7.71 (d, J=8.1, 2H), 7.45 (d, J=8.1 Hz, 2H), 7.25 (d, J=8.3, 1H), 6.37 (d, J=8.4 Hz, 1H), 5.83 (d, J=5.3 Hz, 1H), 4.98 (s, 2H), 4.21-4.26 (m, 2H), 4.09 (s, 1H), 4.01 (s, 2H), 3.08 (d, J=7.0 Hz, 6H), 3.05 (s, 3H), 2.02 (t, J=19.9 Hz, 2H), 1.16 (t, J=6.9 Hz, 9H). ³¹P NMR (D₂O) δ 19.90, 13.50. HRMS calcd C₁₉H₂₅N₄O₁₂P₂ (M−H)⁻: 563.0944; found 563.0950.

N⁴-(4-((2-Aminoethyl)carbamoyl)benzyloxy)-3-methylcytidine-5′-α,β-methylenediphosphate triethylammonium salt (118). The product was obtained as a white solid after lyophilization (4.1 mg, 10.5%, 2-step yield; RP-HPLC acetonitrile/10 mM triethylammonium acetate, 5/100 to 15/85 in 40 min @5 mL/min). ¹H NMR (D₂O, 400 Hz) δ 7.69 (d, J=8.2, 2H), 7.45 (d, J=8.1 Hz, 2H), 7.21 (d, J=8.4, 1H), 6.28 (d, J=8.3 Hz, 1H), 5.80 (d, J=4.8 Hz, 1H), 4.96 (s, 2H), 4.22 (d, J=4.7 Hz, 1H), 4.18 (t, J=4.9 Hz, 1H), 4.01 (s, 2H), 3.60 (t, J=5.7 Hz, 2H), 3.16-3.04 (m, 17H), 2.00 (t, J=18.7 Hz, 2H), 1.17 (t, J=7.3 Hz, 18H). ³¹P NMR (D₂O) δ 20.00, 13.00. HRMS calcd C₂₁H₃₀N₅O₁₂P₂ (M−H)⁻: 606.1366; found 606.1362.

N⁴-(4-((2-Aminobutyl)carbamoyl)benzyloxy)-3-methyleytidine-5′-α,β-methylenediphosphate triethylammonium salt (119). The product was obtained as a white solid after lyophilization (1.8 mg, 8.0%, 2-step yield; RP-HPLC acetonitrile/10 mM triethylammonium acetate, 5/100 to 15/85 in 40 min @5 mL/min). ¹H NMR (D₂O, 400 Hz) δ 7.66 (d, J=8.3, 2H), 7.46 (d, J=8.3 Hz, 2H), 7.27 (d, J=8.3, 1H), 6.36 (d, J=8.3 Hz, 1H), 5.85 (d, J=5.2 Hz, 1H), 4.99 (s, 2H), 4.20-4.28 (m, 2H), 4.12 (s, 1H), 4.03 (d, J=5.0 Hz, 2H), 3.35 (t, J=6.1 Hz, 2H), 3.07-3.15 (m, 4H), 2.95 (t, J=7.1 Hz, 2H), 2.04 (t, J=19.2 Hz, 2H), 1.63 (d, J=3.04 Hz, 4H), 1.19 (t, J=7.2 Hz, 5H). ³¹P NMR (D₂O) δ 19.92, 13.29. HRMS calcd C₂₃H₃₄N₅O₁₂P₂ (M−H)⁻: 634.1679; found 634.1685.

N⁴-(4-(1-(2-Fluoroethyl)-1H-1,2,3-triazol-4-yl)benzyloxy)-3-methylcytidine-5′-α,β-methylenediphosphate triethylammonium salt (120). The product was obtained as a white solid after lyophilization (6.8 mg, 16.0%; RP-HPLC acetonitrile/10 mM triethylammonium acetate, 15/100 to 35/75 in 40 min @5 mL/min). ¹H NMR (D₂O, 400 Hz) δ 8.27 (s, 1H), 7.69 (d, J=7.9, 2H), 7.43 (d, J=7.9 Hz, 2H), 7.22 (d, J=8.3, 1H), 6.34 (d, J=8.2 Hz, 1H), 5.79 (d, J=5.0 Hz, 1H), 4.92 (s, 2H), 4.83 (t, J=4.3 Hz, 2H), 4.70-4.74 (m, 2H), 4.18-4.24 (m, 2H), 4.07 (s, 1H), 4.00 (s, 2H), 3.05 (t, J=5.6 Hz, 15H), 2.00 (t, J=19.6 Hz, 2H), 1.15 (s, 18H). ¹⁹F NMR (D₂O) 6-222.40. ³¹P NMR (D₂O) δ 19.98, 13.02. HRMS calcd C₂₂H₂₈FN₆O₁₁P₂ (M−H)⁻: 633.1275; found 633.1271.

### (((((2R,3S,4R,5S)-4-(2,4-Dioxo-3,4-dihydropyrimidin-1(2H)-yl)-2,3-dihydroxy-1λ⁵-bicyclo[3.1.0]hexan-1-yl)methoxy)(hydroxy)phosphoryl)methyl)phosphonic Acid Triethylammonium Salt (121)

The product was obtained as a white solid after lyophilization (1.6 mg, 6.1%; RP-HPLC acetonitrile/10 mM triethylammonium acetate, 0/100 to 10/90 in 40 min @5 mL/min). ¹H NMR (D₂O, 400 Hz) δ 7.91 (d, J=7.8 Hz, 1H), 5.82 (d, J=7.8 Hz, 1H), 4.28 (dd, J=5.1, 11.0 Hz, 1H), 3.86 (d, J=6.6 Hz, 1H), 3.70 (d, J=11.0 Hz, 1H), 3.60 (dd, J=5.2, 10.5 Hz, 1H), 2.55-3.30 (sbr, 6H), 2.04 (t, J=19.8 Hz, 2H), 1.48 (d, J=7.8 Hz, 1H), 1.14-1.25 (m, 11H), 0.81 (d, J=7.3 Hz, 1H). ³¹P NMR (D₂O) δ 21.00, 13.03. HRMS calcd C₁₂H₁₈N₂O₁₀P₂ (M−H)⁻: 411.0358; found 411.0355.

### (((((2R,3S,4R,5S)-4-((Z)-4-(((4-Chlorobenzyl)oxy)imino)-3-methyl-2-oxo-3,4-dihydropyrimidin-1(2H)-yl)-2,3-dihydroxy-1V-bicyclo[3.1.0]hexan-1-yl)methoxy)(hydroxy)phosphoryl)methyl)phosphonic Acid Triethylammonium Salt (122)

The product was obtained as a white solid after lyophilization (6.9 mg, 16.0%; RP-HPLC acetonitrile/10 mM triethylammonium acetate, 15/100 to 35/65 in 40 min @5 mL/min). ¹H NMR (D₂O, 400 Hz) δ 7.32 (s, 4H), 7.26 (d, J=8.3 Hz, 1H), 6.28 (d, J=8.2 Hz, 1H), 4.87 (s, 2H), 4.53-4.58 (m, 2H), 4.23 (dd, J=5.4, 10.9 Hz, 1H), 3.79 (d, J=6.8 Hz, 1H), 3.64 (dd, J=4.9, 10.9 Hz, 1H), 3.05 (s, 21H), 1.95 (t, J=19.6 Hz, 2H), 1.38 (t, J=4.4 Hz, 2H), 1.15 (s, 27H), 0.75 (t, J=6.6 Hz, 1H). ³¹P NMR (D₂O) δ 22.00, 12.00. HRMS calcd C₂₀H₂₆ClN₃O₁₀P₂ (M−H)⁻: 564.0704; found 564.0702.

4-((Benzylcarbamoyl)imino)-3-methylcytidine-5′-α,β-methylenediphosphate triethylammonium salt (123). The product was obtained as a white solid after lyophilization (7.6 mg, 8.4%; RP-HPLC acetonitrile/10 mM triethylammonium acetate, 15/100 to 35/75 in 40 min @5 mL/min). ¹H NMR (D₂O, 400 Hz) δ 7.64 (d, J=8.2 Hz, 1H), 7.25-7.7.46 (m, 5H), 6.13 (d, J=8.2 Hz, 1H), 5.97 (d, J=3.1 Hz, 1H), 4.42 (s, 2H), 4.35 (s, 2H), 4.24 (s, 1H), 4.14 (s, 2H), 3.31 (s, 3H), 3.19 (dd, J=7.3, 14.6 Hz, 12H), 2.16 (t, J=19.7 Hz, 2H), 1.26 (t, J=7.0 Hz, 18H). ³¹P NMR (D₂O) δ 20.00, 13.00. HRMS calcd C₁₉H₂₅N₄O₁₁P₂ (M−H)⁻: 547.0995; found 547.0995.

### Example 5

This examples demonstrates a rat ecto-5′-nucleotidase assay in accordance with an embodiment of the invention.

The assay was performed as previously described (Williamson et al., J. Med. Chem. 52: 1510-1513 (2009)) using recombinant rat CD73 expressed in Sf9 insect cells (Servos et al., Drug Dev. Res. 45: 269-276 (1998). The assay was performed by adding 10 μL of compound solution to 70 μL of assay buffer (25 mM Tris, 140 mM sodium chloride and 50 mM sodium phosphate, pH 7.4), 10 μL of [³H]AMP (Hartmann Analytic, Germany) with a final substrate concentration of 5 μM and 10 μL, of the enzyme solution (final concentration 0.3 ng/4). The mixture was incubated for 25 min at 37° C. The reaction was stopped by adding 500 μL of ice-cooled precipitation buffer containing 100 mM aqueous lanthanum chloride and 100 mM aqueous sodium acetate solution (pH 4.0). After 30 min, the precipitation was filtered through GF/B glass fiber filters (M-48, Brandel, Gaithersburg, Md., USA). After washing three times, 4 mL of the scintillation cocktail ULTIMA Gold XR was added and then quantified by scintillation counting (TRICARB 2900 TR, Packard/PerkinElmer). Three separate inhibition assays were performed in triplicate, concentration-response curves were fitted, and K, values were calculated.

The results are shown in Tables 1-3.

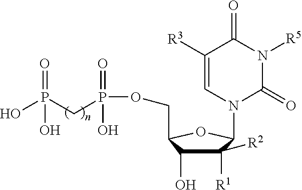

TABLE 1

| | | | | | Kᵢ ± SEM (nM) | |
|---|---|---|---|---|---|---|
| | | | | | (% inhibition at indicated | |
| Compound | R³ | R⁵ | R¹ | R² | concentration), rat CD73 | |
| 4b | H | Me | OH | H | 1864 ± 402 | |
| 4c | H | Et | OH | H | >1000 | (4%) |
| 4d | H | n-Pr | OH | H | >1000 | (7%) |
| 4e | H | Benzyl | OH | H | >1000 | (3%) |
| 4f | Me | H | OH | H | 338 ± 56 | |
| 4g | Me | H | H | H | 639 ± 65 | |
| 4h | Me | H | OMe | H | >1000 | (3%) |
| 4i | Ethynyl | H | OH | H | 276 ± 37 | |
| 4j | 1-chlorovinyl | H | OH | H | 424 ± 27 | |
| 4k | 1-chlorovinyl | Me | OH | H | 1051 ± 291 | |
| 4l | F | H | OH | H | 14.8 ± 1.9 | |
| 4m | Cl | H | OH | H | 86.7 ± 7.6 | |
| 4n | Br | H | OH | H | 88.7 ± 12.5 | |
| 4o | I | H | OH | H | 162 ± 4 | |
| 4p | H | H | H | H | >1000 | (37%) |
| 4q | H | H | NH₂ | H | >1000 | (2%) |
| 4r | H | H | N₃ | H | >1000 | (9%) |
| 4s | H | H | F | H | >1000 | (11%) |
| 4t | H | H | H | H | 1751 ± 378 | |
| 4u | H | H | H | OH | >1000 | (8%) |

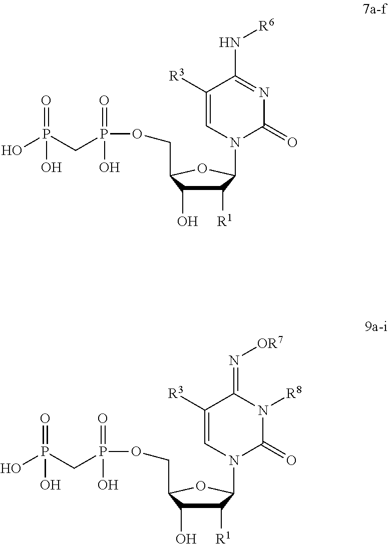

TABLE 2

| | | | | | | Kᵢ ± SEM (nM) |
|---|---|---|---|---|---|---|
| | | | | | | (% inhibition at indicated |
| Compound | R¹ | R³ | R⁶ | R⁷ | R⁸ | concentration), rat CD73 |
| 7a | OH | H | H | — | — | 898 ± 63 |
| 7b | H | H | H | — | — | >1000 (18%) |
| 7c | OH | I | H | — | — | 502 ± 83 |
| 7d | OH | F | H | — | — | 349 ± 41 |
| 7e | OH | Me | H | — | — | 2027 ± 674 |
| 7f | OH | H | PhCO | — | — | 13.9 ± 1.6 |
| 9a | OH | H | — | Me | | 257 ± 39 |
| 9b | OH | H | — | Bn | | 112 ± 15 |
| 9c | H | H | — | Bn | | 780 ± 15 |
| 9d | OH | H | — | 4-CF₃Bn | | 30.3 ± 4.2 |
| 9e | OH | H | — | Naphth-2-yl-methyl | | 18.8 ± 3.2 |
| 9f | OH | Me | — | Bn | | 321 ± 9 |
| 9g | OH | F | — | Bn | | 85.1 ± 7.5 |
| 9h | OH | H | — | Bn | Me | 3.67 ± 0.26 |
| 9i | OH | H | — | Bn | Et | 262 ± 46 |

TABLE 3

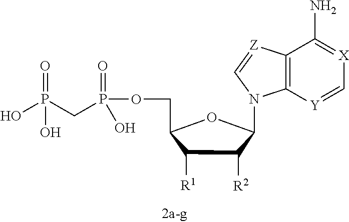

| | | | | Ki ± SEM (nM) |
|---|---|---|---|---|
| | | | | (% inhibition |
| | | | | at indicated |
| | | | | concentration), |
| Compd. | Substitution | R¹ | R² | rat CD73 |
| I, | | OH | OH | 167 ± 53 |
| (AOPCP) | | | | |
| 2a | | OH | H | >1000 (30%) |
| 2b | | OH | NH2 | >1000 (2%) |
| 2c | 5□-OH | OP2O5CH5 | NH2 | >1000 (8%) |
| 2d | | H | OH | 1969 ± 222 |
| 2e | X = CH | OH | OH | >1000 (36%) |
| 2f | Y = CH | OH | OH | >1000 (36%) |
| 2g | Z = CH | OH | OH | 88.6 ± 4.0 |

### Example 5

This examples demonstrates a human ecto-5′-nucleotidase assay in accordance with an embodiment of the invention.

Blood was taken from healthy volunteers by venipuncture and allowed to clot at room temperature for 30 min. Blood samples were centrifuged for 15 min at 2000 g followed by freezing of the serum at −70° C. Serum samples (4-7 μL), or recombinant human CD73 (purified as previously described; Yegutkin et al., Mediators Inflamm. [online] Vol. 2014, Article ID 485743) were incubated for 1 h at 37° C. The enzyme assays were carried out in a final volume of 80 μL of RPMI-1640 medium containing 5 mM β-glycerophosphate, 40 μM of unlabeled AMP containing tracer [2-³H]AMP (18.6 Ci/mmol; Quotient Bioresearch, GE Healthcare) as a substrate. The products were separated by TLC on Alugram SIL G/UV₂₅₄ plates (Machery-Nagel, Duren, Germany) (eluent: 1-butanol 1.5 eq.; isoamyl alcohol, 1 eq.; diethylene glycol monoethyl ether, 3 eq.; ammonia solution, 1.5 eq.; pure water 2.5 eq.) and quantified by scintillation β-counting. Concentration-inhibition curves were performed in 2-4 separate experiments. The results are shown in Table 4.

TABLE 4

| | hP2Y₆ EC₅₀ ± | hP2Y₁₄ IC₅₀ ± | |
|---|---|---|---|
| | SEM (μM) | SEM (μM) | |
| | (% activation at | (% inhibition at | CD73 Kᵢ ± |
| | indicated | indicated | SEM (nM) |
| Compound | concentration) | concentration) | rat eN |
| 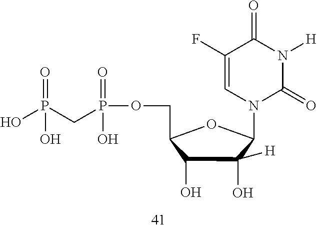 | 0.203 ± 0.030 | 0.362 ± 0.090 | 14.8 ± 1.9 |
| 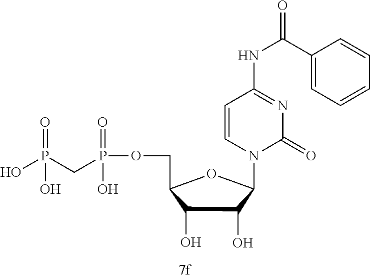 | 1.39 ± 0.22 | 6.66 ± 4.74 | 13.9 ± 1.6 |
| 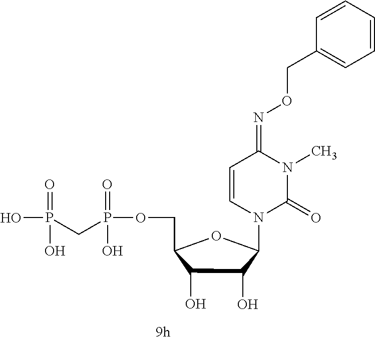 | >3000 (3.6%) | >3000 (inactive) | 3.67 ± 0.26 |

### Example 6

This example demonstrates a comparison of potencies measured with rat and human CD73.

Compounds were assayed against human soluble and membrane-bound CD73 as described herein. The results are set forth in Table 5 and are shown graphically in FIG. 1.

TABLE 5

| | Rat | Human | Human membrane- |
|---|---|---|---|
| | soluble CD73 | soluble CD73 | bound CD73 |
| Compound | Kᵢ ± SEM (nM) | Kᵢ ± SEM (nM) | Kᵢ ± SEM (nM) |
| 4l | 14.8 ± 1.9 | 5.33 ± 0.73 | 4.51 ± 0.13 |
| 7f | 13.9 ± 1.6 | 4.58 ± 0.55 | 5.68 ± 0.75 |
| 9d | 30.3 ± 4.2 | 14.0 ± 1.6 | 10.1 ± 1.4 |
| 9e | 18.8 ± 3.2 | 6.88 ± 1.05 | 6.29 ± 0.45 |
| 9g | 85.1 ± 7.5 | 15.9 ± 1.1 | 16.6 ± 0.7 |
| 9h | 3.67 ± 0.26 | 10.6 ± 0.4 | 7.96 ± 0.57 |

As is apparent from the results set forth in Table 5, the inventive compounds are usually slightly more potent at human CD73 as compared to the rat enzyme. Compounds 41, 7f, 9d and 9e are 2 to 3-fold more potent while 9g is 5-fold more potent. However, the most potent compound at rat CD73 (9h, 3.67 nM) is somewhat less potent at the human enzyme (Kᵢ, soluble CD73, 10.6 nM; membrane preparation, 7.96 nM). All tested inhibitors were similarly potent at soluble CD73 as compared to the membrane-bound enzyme.

### Example 6

This example demonstrates activities of inventive compounds in a human ecto-5′-nucleotidase assay, in accordance with an embodiment of the invention.

Compounds were assayed against human soluble CD73 as described herein. The results are set forth in Table 6.

TABLE 6

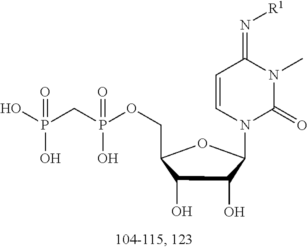

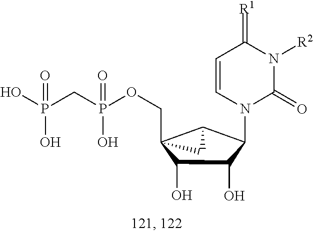

| Compound | | Kᵢ at hCD73 |
|---|---|---|
| no. | R¹ | (nM) |
| 104 | 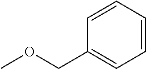 | 10.6 ± 0.4^{a} |
| 105 | 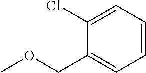 | 1.63 ± 0.28 |
| 106 | 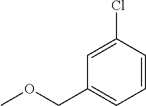 | 1.61 ± 0.09 |
| 107 | 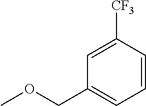 | 1.44 ± 0.09 |
| 108 | 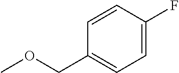 | 1.14 ± 0.06 |
| 109 | 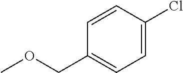 | 0.673 ± 0.091 |
| 110 | 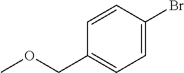 | 0.441 ± 0.157 |
| 111 | 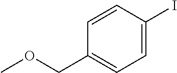 | 0.436 ± 0.078 |
| 112 | 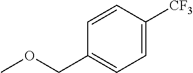 | 1.08 ± 0.34 |
| 113 | 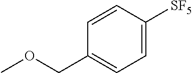 | 0.511 ± 0.041 |
| 114 | 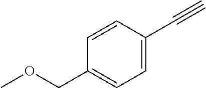 | 0.664 ± 0.089 |
| 115 | 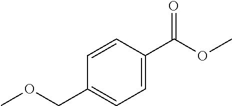 | 0.848 ± 0.229 |
| 116 | 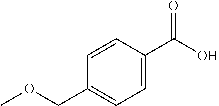 | 14.1 ± 2.9 |
| 117 | 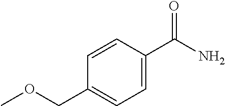 | 1.76 ± 0.43 |
| 118 | 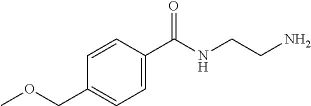 | 0.626 ± 0.076 |
| 119 | 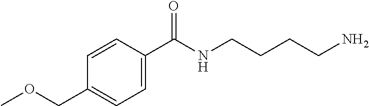 | 1.78 ± 0.33 |
| 120 | 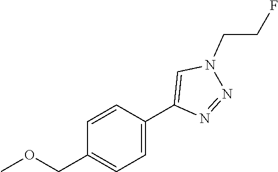 | 0.627 ± 0.099 |
| 121 | O, R² = H | 35% inhibition |
| | | at 50 μM |
| 122 | 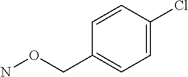 | 59.2 ± 5.58 |
| 123 | 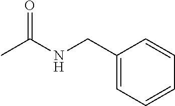 | 71.5 ± 17.6 |

### Example 7

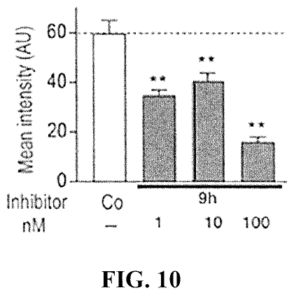

This example demonstrates the inhibition of human tonsillar CD73-mediated AMPase activity by compound 9h, in accordance with an embodiment of the invention. Palatine tonsils were obtained from adult patients with chronic tonsillitis undergoing routine tonsillectory. The tonsils were washed with phisiological salt solution, embedded in the cyro-mold with Tissue-Tek O.C.T. compound (Sakura Finetek Europe B.V., the Nethelands), cut using a cryostat and stored at −80° C. Tonsil cryoselctions were preincubated for 30 min in Trizma maleate sucrose buffer supplemented with the alkaline phosphatase inhibitor levamisole and different concentrations of compound 9h. The enzymatic reaction was then performed for 45 min at 37° C. in a final volume of 20 mL of TMSB-buffered substrate solution containing Pb (NO3)2, CaCl2, and tested CD73 inhibitor at the same concentration. The lead orthophosphate precipitated in the course of nucleotidase activity was visualized as a brown deposit by incubating sections in (NH3)2S for 30s, followed by three washed in Trizma-maleate buffer for 5 min each. Slides were mounted with Aquatex medium (Merck, Germany). Multiple images of adjacent tissue areas were captured using a slide scanner and further stitched to a larger overview. The images of control and treated tissues were captured at identical exposure times and other settings and further acquired in parallel. Nucleotidase activities were quantified as mean pixel intensities after grayscale conversion. The mean intensity (correlated with CD-73 activity) versus concentration for control and for compound 9h is shown in FIG. 10.

As is apparent from the results shown in FIG. 10, compound 9h at 1, 10, and 100 nM concentrations inhibited the catalytic activity of CD73 in a concentration-dependent manner.

### REFERENCES

1. Zimmermann, H. 5′-Nucleotidase: molecular structure and functional aspects. Biochem. J 1992, 285 (Pt 2), 345-365.
2. Sträter, N. Ecto-5′-nucleotidase: Structure function relationships. Purinergic Signalling 2006, 2 (2), 343-350.
3. Knapp, K.; Zebisch, M.; Pippel, J.; El-Tayeb, A.; Müller, Christa E.; Sträter, N. Crystal structure of the human ecto-5′-nucleotidase (CD73): Insights into the regulation of purinergic signaling. Structure 2012, 20 (12), 2161-2173.
4. Freundlieb, M.; Zimmermann, H.; Müller, C. E. A new, sensitive ecto-5′-nucleotidase assay for compound screening. Anal. Biochem. 2014, 446, 53-58.
5. Augusto E.; Matos M.; Sévigny J.; El-Tayeb A.; Bynoe M S.; Müller C E.; Cunha R A.; Chen J F. Ecto-5′-nucleotidase (CD73)-mediated formation of adenosine is critical for the striatal adenosine A2A receptor functions. J. Neurosci. 2013, 33, 11390-11399. doi: 10.1523/JNEUROSCI.5817-12.2013.
6. Flögel, U.; Burghoff, S.; van Lent, P L.; Temme, S.; Galbarz, L.; Ding, Z.; El-Tayeb, A.; Huels, S.; Bönner, F.; Borg, N.; Jacoby, C.; Müller, C E.; van den Berg, W. B.; Schrader, J. Selective activation of adenosine A2A receptors on immune cells by a CD73-dependent prodrug suppresses joint inflammation in experimental rheumatoid arthritis. Sci. Transl. Med. 2012, 4, 146ra108. doi: 10.1126/scitranslmed.3003717.
7. Corbelini, P. F.; Figueir, F.; Machado das Neves, G.; Andrade, S.; Kawano, D. F., Oliveira Battastini, A. M.; Eifler-Lima, V. L. Insights into ecto-5′-nucleotidase as a new target for cancer therapy: A medicinal chemistry study. Curr. Med. Chem. 2015, 22, 1776-1792.
8. Xu, S.; Shao, Q. Q.; Sun, J. T.; Yang, N.; Xie, Q.; Wang, D. H.; Huang, Q. B.; Huang, B.; Wang, X. Y.; Li, X. G.; Qu, X. Synergy between the ectoenzymes CD39 and CD73 contributes to adenosinergic immunosuppression in human malignant gliomas. Neuro. Oncol. 2013, 15, 1160-1172.
9. Bhattarai, S.; Freundlieb M.; Pippel J.; Meyer A.; Abdelrahman A.; Fiene A.; Lee S. Y.; Zimmermann H.; Yegutkin G. G.; Sträter N.; El-Tayeb A.; Müller C. E. α,β-Methylene-ADP (AOPCP) Derivatives and Analogues: Development of Potent and Selective ecto-5′-Nucleotidase (CD73) Inhibitors. J. Med. Chem. 2015, 58, 6248-6263.
10. Heuts, D. P. H. M.; Weissenborn, M. J.; Olkhov, R. V.; Shaw, A. M.; Gummadova, J.; Levy, C.; Scrutton, N. S. Crystal structure of a soluble form of human CD73 with ecto-5′-nucleotidase activity. ChemBioChem. 2012, 13, 2384-2391.
11. Stagg, J.; Divisekera, U.; McLaughlin, N.; Sharkey, J.; Pommey, S.; Denoyer, D.; Dwyer, K. M.; Smyth, M. J. Anti-CD73 antibody therapy inhibits breast tumor growth and metastasis. Proc. Natl. Acad. Sci. 2010, 107, 1547-1552.
12. Yegutkin, G. G. Enzymes involved in metabolism of extracellular nucleotides and nucleosides: functional implications and measurement of activities. Crit. Rev. Biochem. Mol. Biol., 2014, 49, 473-497.
13. Yegutkin, G. G.; Marttila-Ichihara, F.; Karikoski, M.; Niemela, J.; Laurila, J. P.; Elima, K.; Jalkanen, S.; Salmi, M. Altered purinergic signaling in CD73-deficient mice inhibits tumor progression. Eur. J. Immunol. 2011, 41, 1231-1241.
14. Iqbal, J.; Jirovsky, D.; Lee, S.-Y.; Zimmermann, H.; Müller, C. E. Capillary electrophoresis-based nanoscale assays for monitoring ecto-5′-nucleotidase activity and inhibition in preparations of recombinant enzyme and melanoma cell membranes. Anal. Biochem. 2008, 373, 129-140.
15. Baqi, Y.; Lee, S.-Y.; Iqbal, J.; Ripphausen, P.; Lehr, A.; Scheiff, A.; Zimmermann, H.; Bajorath, J.; Müller, C. Development of potent and selective inhibitors of ecto-5′-nucleotidase based on an anthraquinone scaffold. J. Med. Chem. 2010, 53, 2076-2086.
16. Ripphausen, P.; Freundlieb, M.; Brunschweiger, A.; Zimmermann, H.; Müller, C. E.; Bajorath, J. Virtual screening identifies novel sulfonamide inhibitors of ecto-5′-nucleotidase. Med. Chem. 2012, 55, 6576-6581.
17. Lee, S.-Y.; Fiene, A.; Li, W.; Hanck, T.; Brylev, K. A.; Fedorov, V. E.; Lecka, J.; Haider, A.; Pietzsch, H.-J.; Zimmermann, H.; Sévigny, J.; Kortz, U.; Stephan, H.; Müller, C. E. Polyoxometalates—potent and selective ecto-nucleotidase inhibitors. Biochem. Pharmacol. 2015, 93, 171-181.
18. Beavis, P. A.; Stagg, J.; Darcy, P. K.; Smyth, M. J. CD73: a potent suppressor of antitumor immune responses. Trends Immunol. 2012, 33, 231-237.
19. al-Rashida, M.; Iqbal, J. Therapeutic potentials of ecto-nucleoside triphosphate diphosphohydrolase, ecto-nucleotide pyrophosphatase/phosphodiesterase, ecto-5′-nucleotidase, and alkaline phosphatase inhibitors. Med. Res. Rev. 2014, 34, 703-743.
20. Sociali G, Raffaghello L, Magnone M, Zamporlini F, Emionite L, Sturla L, Bianchi G, Vigliarolo T, Nahimana A, Nencioni A, et al. Antitumor effect of combined NAMPT and CD73 inhibition in an ovarian cancer model. Oncotarget 2016, 7, 2968-2984.
21. Antonioli, L.; Yegutkin, G. G.; Pacher, P.; Blandizzi, C.; Haskó, G. Anti-CD73 in cancer immunotherapy: awakening new opportunities. Trends Cancer 2016, 2, 95-109.
22. Cekic, C.; Linden, J. Purinergic regulation of the immune system. Nature Rev. Immunol. 2016, 16, 177-192.
23. Bruns, R. F. Adenosine receptor activation by adenine nucleotides requires conversion of the nucleotides to adenosine. Naunyn Schmiedebergs Arch. Pharmacol. 1980; 315(1):5-13.
24. Braganhol, E.; Tamajusuku, A. S.; Bernardi, A.; Wink, M. R.; Battastini, A. M. Ecto-5′-nucleotidase/CD73 inhibition by quercetin in the human U138MG glioma cell line. Biochim. Biophys. Acta 2007, 1770, 1352-1359.
25. Lee, S.-Y.; Fiene, A.; Li, W.; Hanck, T.; Brylev, K. A.; Fedorov, V. E.; Lecka, J.; Haider, A.; Pietzsch, H.-J.; Zimmermann, H.; Sévigny, J.; Kortz, U.; Stephan, H.; Müller, C. E. Polyoxometalates—potent and selective ecto-nucleotidase inhibitors. Biochem. Pharmacol. 2015, 93, 171-181.
26. Sadej, R.; Spychala, J.; Skladanowski, A. C. Expression of ecto-5′-nucleotidase (eN, CD73) in cell lines from various stages of human melanoma. Melanoma Res. 2006, 16, 213-222.
27. Wang, L.; Fan, J.; Thompson, L. F.; Zhang, Y.; Shin, T.; Curiel, T. J.; Zhang, B. CD73 has distinct roles in nonhematopoietic and hematopoietic cells to promote tumor growth in mice. J Clin. Invest. 2011, 121, 2371-2382.
28. Loi, S.; Pommey, S.; Haibe-Kains, B.; Beavis, P. A.; Darcy, P. K.; Smyth, M. J.; Stagg, J. CD73 promotes anthracycline resistance and poor prognosis in triple negative breast cancer. Proc. Natl. Acad. Sci. U.S. A. 2013, 110, 11091-11096.
29. Jin, D.; Fan, J.; Wang, L.; Thompson, L.; Liu, A.; Daniel, B.; Shin, T.; Curiel, T.; Zhang, B. CD73 on tumor cells impairs antitumor T-cell responses: a novel mechanism of tumor-induced immune suppression. Cancer Res. 2010, 70, 2245-2255.
30. Zhou, X.; Zhi, X.; Zhou, P.; Chen, S.; Zhao, F.; Shao, Z.; Ou, Z.; Yin, L. Effects of ecto-5′-nucleotidase on human breast cancer cell growth in vitro and in vivo. Oncol. Rep. 2007, 17, 1341-1346.
31. Hay, C. M.; Sult, E.; Huang, Q.; Mulgrew, K.; Fuhrmann, S. R.; McGlinchey, K. A.; Hammond, S. A.; Rothstein, R.; Rios-Doria, J.; Poon, E.; Holoweckyj, N.; Durham, N. M.; Leow, C. C.; Diedrich, G.; Damschroder, M.; Herbst, R.; Hollingsworth, R. E.; Sachsenmeier, K. F. Targeting CD73 in the tumor microenvironment with MEDI9447. Oncoimmunology. 2016, 5, el 208875.
32. MEDI9447 Alone and in Combination With MEDI4736 in Adult Subjects With Select Advanced Solid Tumors. https://clinicaltrials.gm/ct2/show/NCT02503774, accessed Jul. 23, 2018.
33. Servos, J.; Reilander, H.; Zimmermann, H. Catalytically active soluble ecto-5′-nucleotidase purified after heterologous expression as a tool for drug screening. Drug Dev. Res. 1998, 45, 269-276.
34. Freundlieb, M.; Zimmermann, H.; Müller, C. E. A new, sensitive ecto-5′-nucleotidase assay for compound screening. Anal. Biochem. 2014, 446, 53-58.
35. Altschul, S. F.; Madden, T. L.; Schäffer, A. A.; Zhang, J.; Zhang, Z.; Miller, W.; Lipman, D. J. Gapped BLAST and PSI-BLAST: Anew generation of protein database search programs. Nucleic Acids Res. 1997, 25, 3389-3402.
36. Altschul, S. F.; Wootton, J. C.; Gertz, E. M.; Agarwala, R.; Morgulis, A.; Schäffer, A. A.; Yu, Y.-K. Protein database searches using compositionally adjusted substitution matrices. FEBS J. 2005, 272, 5101-5109.
37. Loi, S.; Pommey, S.; Haibe-Kains, B.; Beavis, P. A.; Darcy, P. K.; Smyth, M. J.; Stagg, J. CD73 promotes anthracycline resistance and poor prognosis in triple negative breast cancer. Proc Natl Acad Sci USA. 2013, 110, 11091-11096.
38. Terp, M. G.; Olesen, K. A.; Arnspang, E. C.; Lund, R. R.; Lagerholm, B. C.; Ditzel, H. J.; Leth-Larsen, R. Anti-human CD73 monoclonal antibody inhibits metastasis formation in human breast cancer by inducing clustering and internalization of CD73 expressed on the surface of cancer cells. J. Immunol. 2013, 191, 4165-4173.
39. Young, A.; Ngiow, S. F.; Barkauskas, D. S.; Sult, E.; Hay, C.; Blake, S. J.; Huang, Q.; Liu, J.; Takeda, K.; Teng, M. W. L.; Sachsenmeier, K.; Smyth, M. J. Co-inhibition of CD73 and A2A R Adenosine Signaling Improves Anti-tumor Immune Responses. Cancer Cell. 2016, 30, 391-403.
40. Allard, D.; Chrobak, P.; Allard, B.; Messaoudi, N.; Stagg, J. Targeting the CD73-adenosine axis in immuno-oncology. Immunol Lett. 2018, https://doi.org/10.1016/j.imlet.2018.05.001.
41. Williamson, D. S.; Borgognoni, J.; Clay, A.; Daniels, Z.; Dokurno, P.; Drysdale, M. J.; Foloppe, N.; Francis, G. L.; Graham, C. J.; Howes, R.; Macias, A. T.; Murray, J. B.; Parsons, R.; Shaw, T.; Surgenor, A. E.; Terry, L.; Wang, Y.; Wood, M.; Massey, A. J. Novel adenosine-derived inhibitors of 70 kDa heat shock protein, discovered through structure-based design. J. Med. Chem. 2009, 52, 1510-1513.
42. Yamamoto, I.; Kimura, T.; Tateoka, Y.; Watanabe, K.; Ho, I. K. N-Substituted oxopyrimidines and nucleosides: structure-activity relationship for hypnotic activity as CNS depressant. J. Med. Chem. 1987, 30, 2227-2231.
43. Jayasekara, P. S.; Barrett, M. O.; Ball, C. B.; Brown, K. A.; Kozma, E.; Costanzi, S.; Squarcialupi, L.; Balasubramanian, R.; Maruoka, H.; Jacobson, K. A. 4-Alkyloxyimino-cytosine nucleotides: tethering approaches to molecular probes for the P2Y₆ receptor. MedChemComm 2013, 4, 1156-1165.
44. Sako, M.; Kawada, H. A New and Efficient Synthetic Method for ¹⁵N₃-Labeled Cytosine Nucleosides: Dimroth Rearrangement of Cytidine N₃-Oxides J. Org. Chem. 2004, 69, 8148-8150.

All references, including patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula (I): 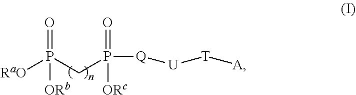
wherein Q is O, S, CH₂, or NH;
U is O, CH₂, C₂H₄, NH or S;
n is an integer of from 1 to 6;
R^{a}, R^{b} and R^{c} are independently H, C₁-C₆ alkyl, C₆-C₁₀ aryl, C₁-C₆ alkylcarbonyl, C₆-C₁₀ heteroaryl-C₁-C₆ alkyl, C₆-C₁₀ heteroaryl, or C₆-C₁₀ arylcarbonyl;
T is 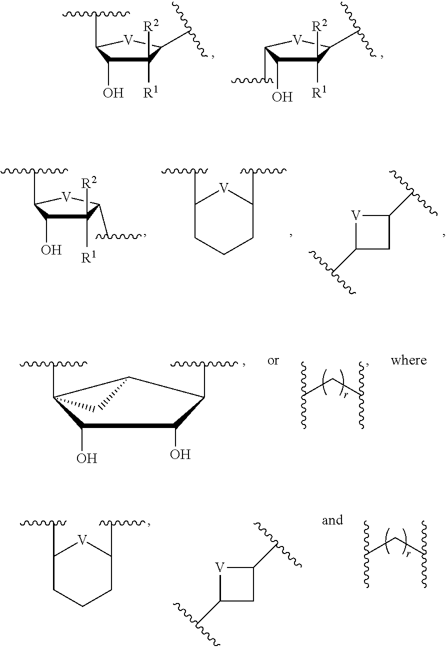
are optionally substituted with 1, 2, or 3 hydroxyl groups and r is an integer from 2 to about 6,
V is O, S, CH₂ or NH;
A is 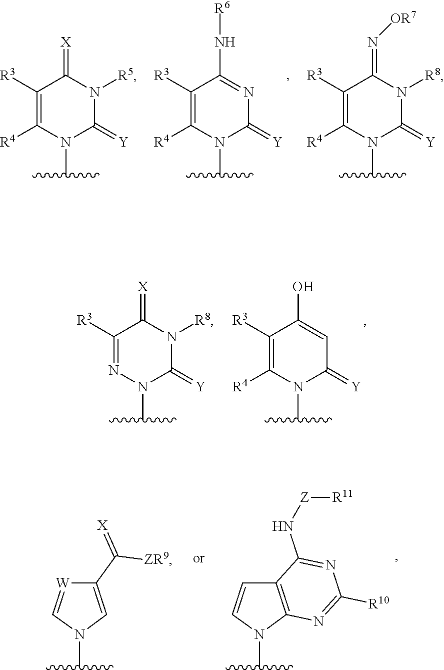
wherein X, Y, and Z are independently O, S, NH or C₁-C₆ alkylenyl, and W is independently N or CH,
R¹ and R² are independently H, OH, SH, C₁-C₆ alkoxy, C₁-C₆ thioalkoxy, NH₂, C₁-C₆ alkylamino, N₃, or halo;
R³, R⁴ and R¹⁰ are independently H, halo, C₁-C₆ alkyl, C₁-C₆ aryl, NH₂, N₃, C₂-C₆ alkynyl, C₆-C₁₀ aryl-C₁-C₆ alkylenyl, 1-halo-1-vinyl, C₆-C₁₀ heteroaryl-C₁-C₆ alkyl, C₆-C₁₀ heteroaryl or C₆-C₁₀ arylcarbonyl,
R⁵ to R⁸ and RH are independently H, C₁-C₆ alkyl, C₆-C₁₀ aryl-C₁-C₆ alkyl, C₆-C₁₀ aryl-C₁-C₆ alkylenyl, C₁-C₆ alkylcarbonyl, C₆-C₁₀ heteroaryl-C₁-C₆ alkyl, C₆-C₁₀ heteroaryl or C₆-C₁₀ arylcarbonyl,
R⁹ is C₁-C₆ alkyl, C₆-C₁₀ aryl-C₁-C₆ alkylenyl, C₁-C₆ alkylcarbonyl, C₆-C₁₀ heteroaryl-C₁-C₆ alkyl, C₆-C₁₀ heteroaryl or C₆-C₁₀ arylcarbonyl,
wherein aryl at each occurrence is optionally substituted with one or more substituents selected from the group consisting of C₁-C₆ alkyl, C₂-C₆ alkynyl, halo, trifluoromethyl, OH, SH, NH₂, SO₂NH₂, C₁-C₆ hydroxyalkyl, C₁-C₆ alkoxy, sulfonyloxy, C₁-C₆ carboxyalkyl, carboxy, carboxamide, C₁-C₆ sulfonyloxyalkyl, arylcarbonyl, CONH(CH)ₚNH₂, 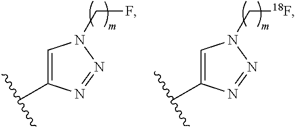
and any combination thereof, wherein heteroaryl is optionally substituted with one or more substituents selected from C₁-C₆ alkyl, halo, trifluoromethyl, C₁-C₆ hydroxyalkyl, C₁-C₆ alkoxy, sulfonyloxy, C₁-C₆ carboxyalkyl, C₁-C₆ sulfonyloxyalkyl, arylcarbonyl, 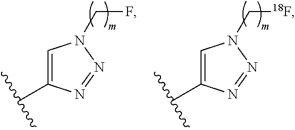
and any combination thereof,
wherein m is an integer of from 2 to about 10,
wherein p is an integer of from 2 to about 10,
with the provisos that when R^{a}, R^{b}, and R^{c} are all H, n is 1, Q is O, U is CH₂, T is 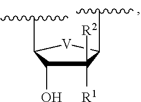
V is O, W is OH, R² is H, and A is 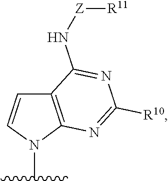
R⁵ is not H,
methyl, ethyl, or benzyl and R¹⁰ is not H,
or a pharmaceutically acceptable salt thereof.

2. The compound or salt of claim 1, wherein Q is O, U is CH₂, T is 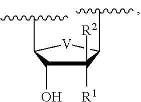
A is 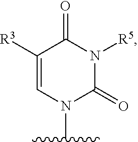
R¹ is H, OH, NH₂, N₃, C₁-C₆ alkoxy or halo and R² is H or halo, R³ is H, halo, methyl, ethynyl, or 1-chloro-1-vinyl, and R⁵ is H, methyl, ethyl, propyl or benzyl.

3.-6. (canceled)

7. The compound or salt of claim 2, wherein the compound is selected from the group consisting of:

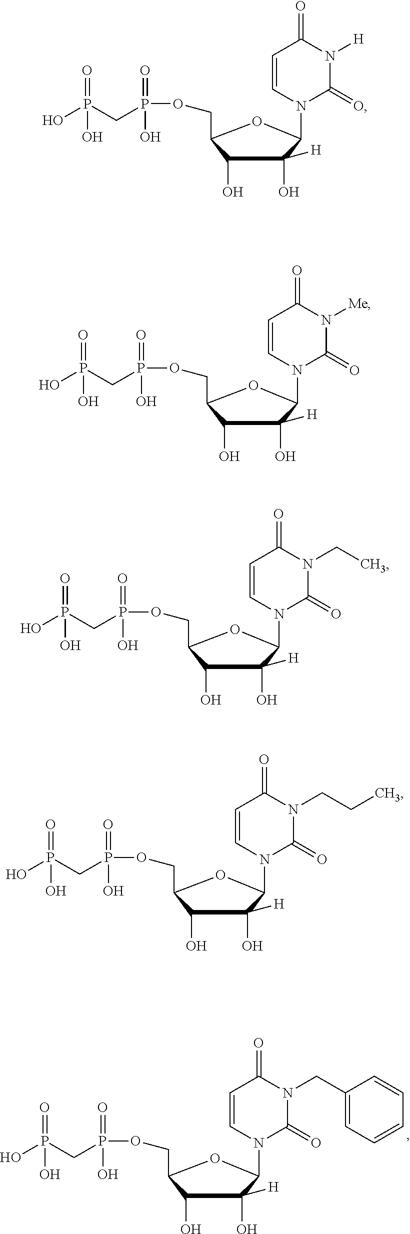

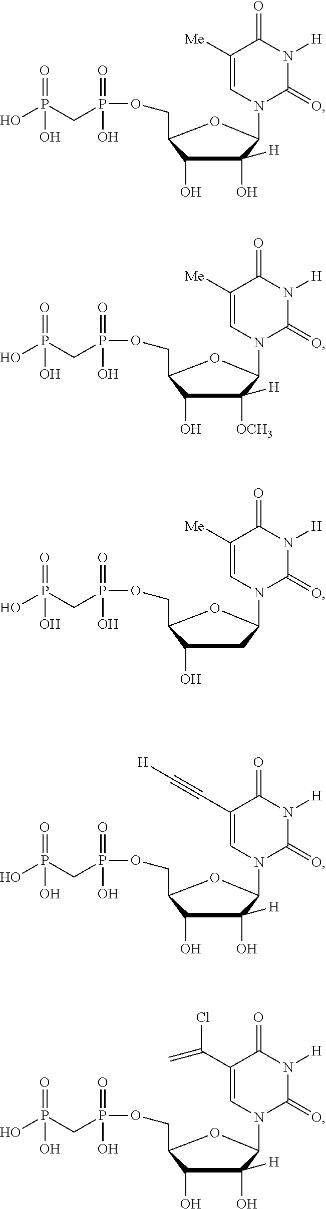

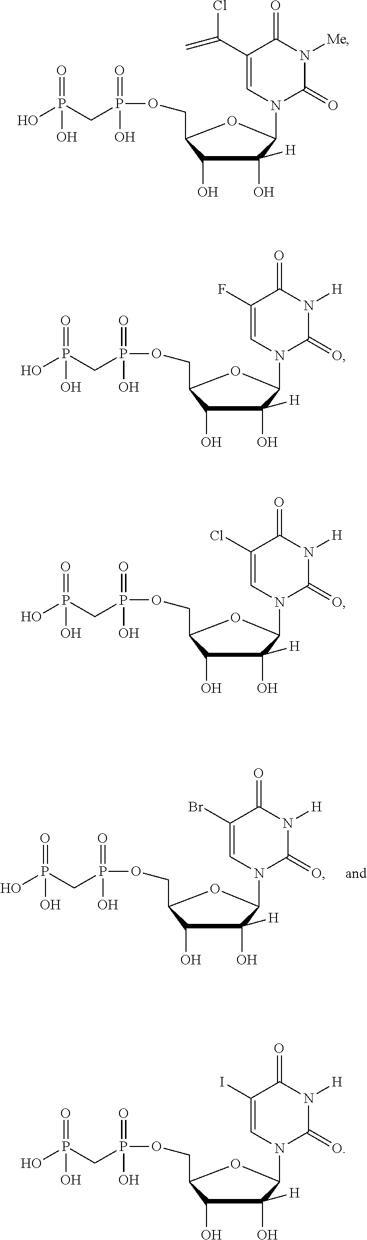

8. The compound or salt of claim 2, wherein R¹ is H and R² is F or OH.

9. The compound or salt of claim 8, wherein the compound is selected from the group consisting of:

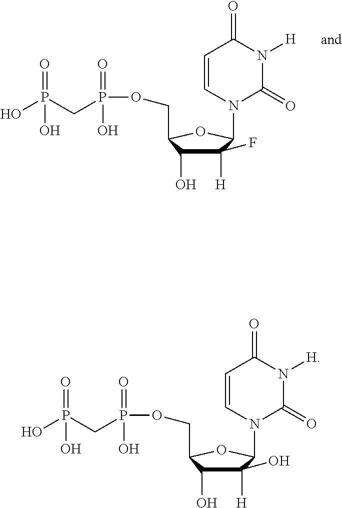

10. The compound or salt of claim 1, wherein T is 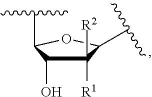
A is 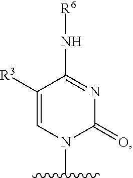
R¹ is OH or H, R² is H, R³ is H, halo, or C₁-C₆ alkyl, and R⁶ is H, C₁-C₆ alkyl, C₁-C₆-alkylcarbonyl, or C₆-C₁₀ arylcarbonyl.

11.-13. (canceled)

14. The compound or salt of claim 10, wherein the compound is selected from the group consisting of:

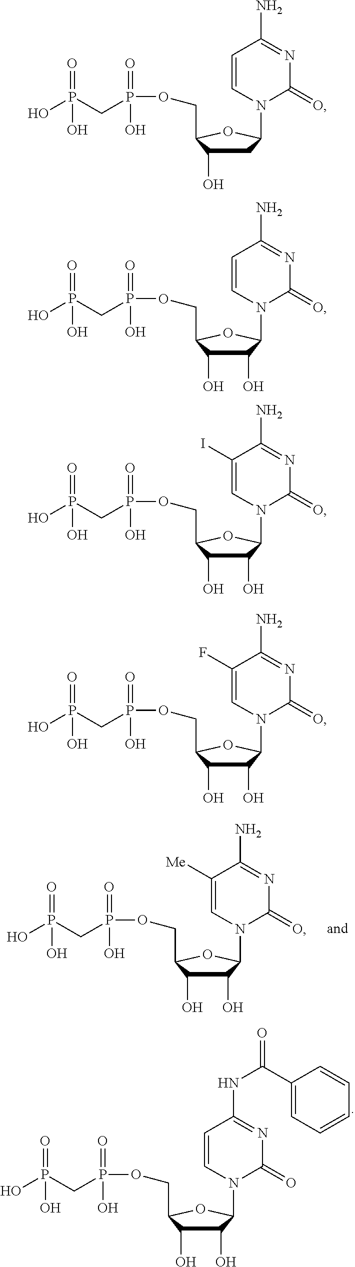

15. The compound or salt of claim 1, wherein T is 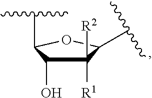
A is 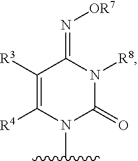
R¹ is H or OH, R² is H, R³ is H, halo or C₁-C₆ alkyl, R⁷ is H, C₁-C₆ alkyl, C₆-C₁₀ aryl-C₁-C₆ alkyl, C₆-C₁₀ aryl-C₁-C₆ alkylenyl, C₁-C₆ alkylcarbonyl, C₆-C₁₀ heteroaryl-C₁-C₆ alkyl, C₆-C₁₀ heteroaryl or C₆-C₁₀ arylcarbonyl, and R⁸ is C₁-C₆ alkyl or C₆-C₁₀ aryl-C₁-C₆ alkylenyl.

16.-19. (canceled)

20. The compound or salt of claim 15, wherein the compound is selected from the group consisting of:

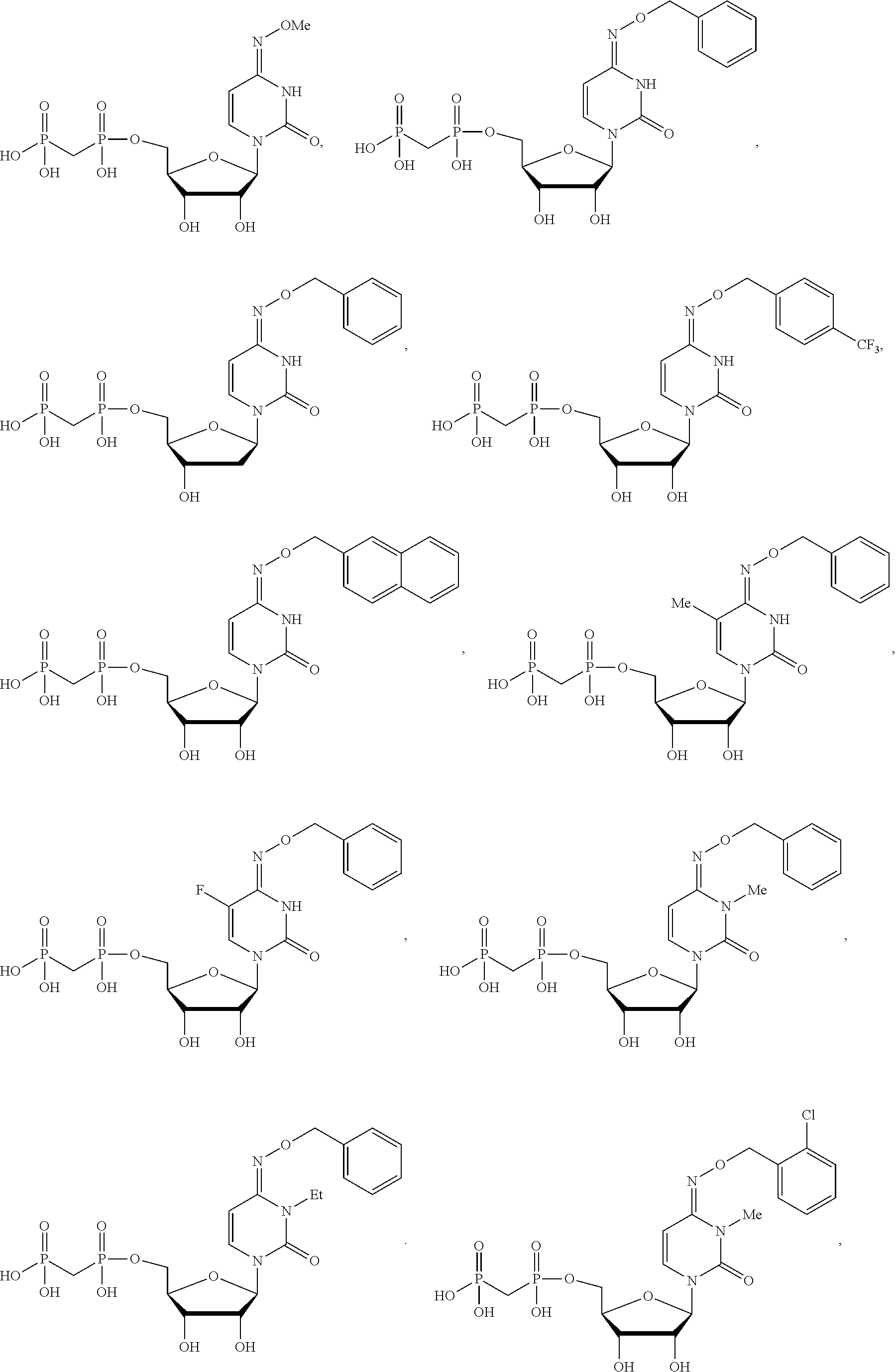

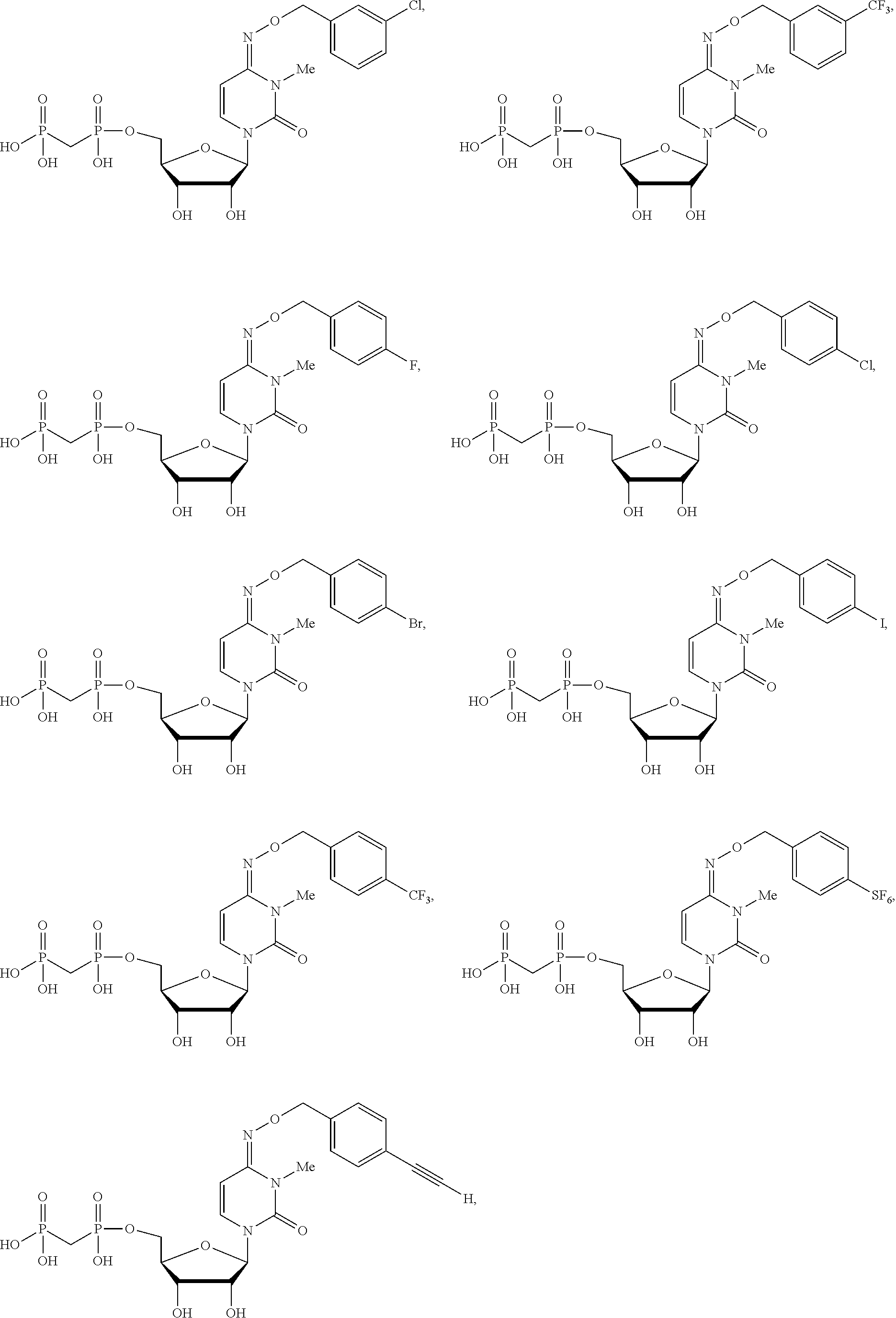

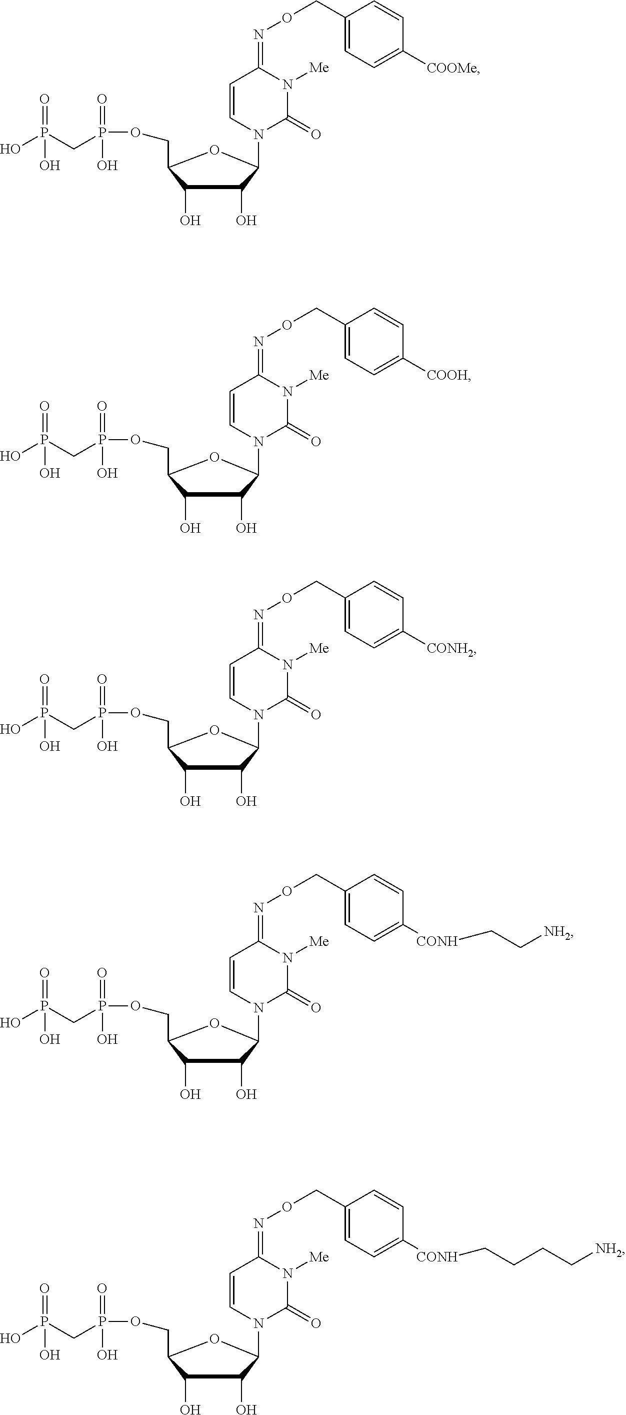

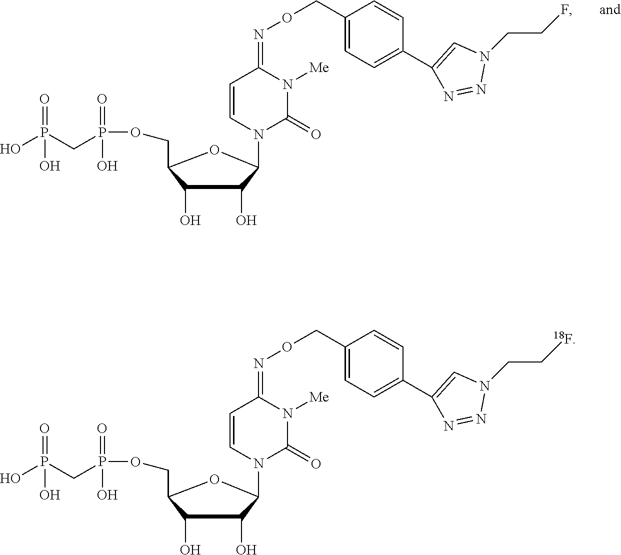

21. The compound or salt of claim 1, wherein T is 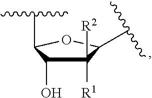
A is 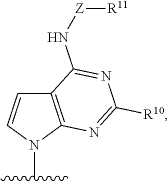
R¹ is OH and R² is H, R¹⁰ is H, R⁵ is C₆-C₁₀ aryl, and Z is CH₂ or C₂H₄.

22.-25. (canceled)

26. The compound or salt of claim 21, wherein the compound is selected from the group consisting of:

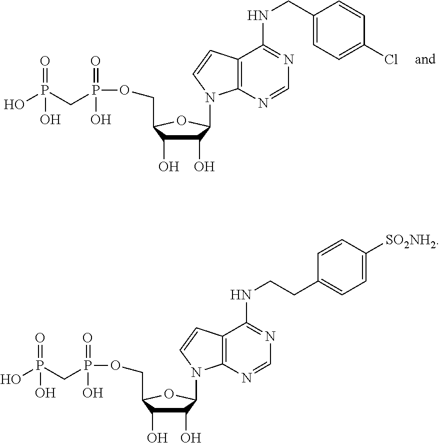

27. (canceled)

28. The compound or salt of claim 1, wherein the compound is:

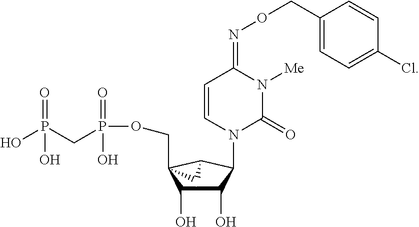

29. A pharmaceutical composition comprising the compound or salt of claim 1 and a pharmaceutically acceptable carrier.

30. A method of inhibiting ecto-5′-nucleotidase in a mammal in need thereof, (b) inhibiting suppression of an antitumor immune response in a mammal in need thereof, (c) inhibiting tumor growth of a cancerous tumor in a mammal in need thereof, (d) inhibiting metastasis of cancer in a mammal afflicted with cancer, (e) synergistically enhancing a response of a mammal afflicted with cancer undergoing treatment with an immunotherapeutic anti-cancer agent, (f) potentiating an activity of an inhibitor of nicotinamide phosphoribosyltransferase in a mammal undergoing treatment of a mammal with the inhibitor, (g) treating preeclampsia in in a mammal in need thereof, or (h) treating cancer in a mammal, wherein the mammal in (a)-(f) and (h) is afflicted with lung cancer, prostate cancer, triple-negative breast cancer, melanoma, leukemia, or thyroid cancer or wherein the tumor is associated with lung cancer, prostate cancer, triple-negative breast cancer, or thyroid cancer, comprising administering to the mammal an effective amount of a compound or salt of claim 1.

31.-40. (canceled)

41. The method of claim 30, wherein the method synergistically enhances a response of a mammal afflicted with cancer undergoing treatment with an immunotherapeutic anti-cancer agent, and wherein the immunotherapeutic anti-cancer agent is selected from the group consisting of pembrolizumab, nivolumab, atezolizumab, durvalumab, and ipilimumab.

42.-45. (canceled)

46. A method of imaging a mammal by positron emission tomography (PET), comprising administering to the mammal a compound of claim 1, wherein the compound is: 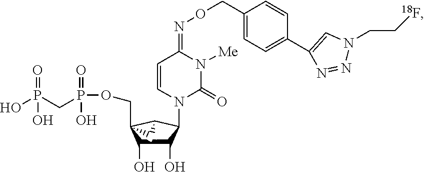
and imaging the mammal.

47. A compound of the formula: 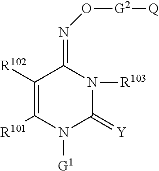
wherein R¹⁰¹ and R¹⁰² are independently H, halo, C₁-C₆ alkyl, C₁-C₆ aryl, NH₂, N₃, C₁-C₆ alkynyl, C₆-C₁₀ aryl-C₁-C₆ alkylenyl, 1-halo-1-vinyl, C₆-C₁₀ heteroaryl-C₁-C₆ alkyl, C₆-C₁₀ heteroaryl or C₆-C₁₀ arylcarbonyl,
R¹⁰³ is H, C₁-C₆ alkyl, C₆-C₁₀ aryl-C₁-C₆ alkyl, C₆-C₁₀ aryl-C₁-C₆ alkylenyl, C₁-C₆ alkylcarbonyl, C₆-C₁₀ heteroaryl-C₁-C₆ alkyl, C₆-C₁₀ heteroaryl or C₆-C₁₀ arylcarbonyl,
G¹ is 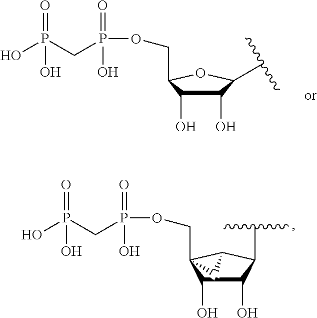
G² is 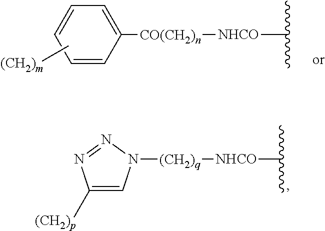
m, n, p, and q are independently integers of from 1 to about 20, and
Q is a fluorophore moiety,
wherein Q is a fluorophore moiety selected from the group consisting of FITC, NBD, Dansyl, Squaraine Rotaxane, Bodipy FL, Bodipy TR, Bodipy 630/650-X, Bodipy 650/655-X, Texas Red, Cy5, 1-pyrene, EVOBlue 30, Alexa Fluor 532, Alexa Fluor 488-5, 488-6, or mixture thereof, Tamra, Tamra 5/6-X-SE, Alexa Fluor 488 azide 5 isomer, Alexa Fluor 488 5 isomer, Alexa Fluor 488 5/6 mixed isomers, NIR dye 700, NIR dye 800, Janelia Fluor 549 amide, and Janelia Fluor 646 amide;
or a pharmaceutically acceptable salt thereof.

48.-50. (canceled)

51. The compound or salt of claim 47, wherein the compound is:

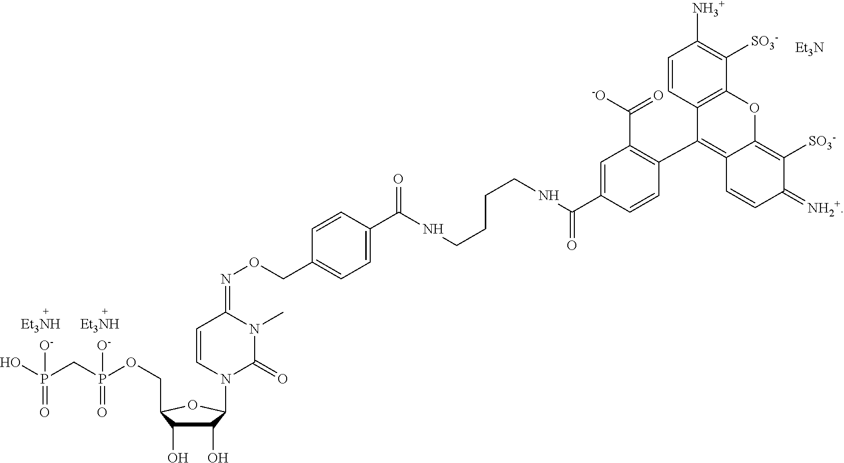

52. (canceled)

53. The compound or salt of claim 47, wherein the compound is:

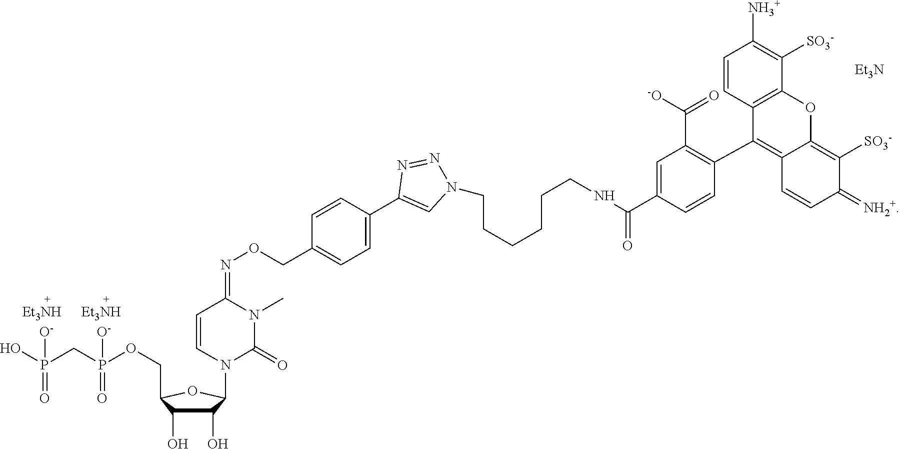

54. A diagnostic composition comprising a compound or salt of claim 47 and a pharmaceutically acceptable carrier.
